SEQUENCE LISTING (204 sequences)

SEQ ID NO: 1 - DNA, 2532 bp, Hepatitis B virus Type B
atgcccctatcttatcaacacttccggaaactactgttgttagacgaagaggcaggtcccctagaagaagaactccctcgcctcgcagacgaaggtctcaatcgccgcgtcgcagaagatctcaatctcgggaatctcaatgttagtattccttggacacataaggtgggaaactttacggggctttattcttctacggtaccttgctttaatcctaaatggcaaactccttcttttcctgacattcatttgcaggaggacattgttgatagatgtaagcaatttgtggggccccttacagtaaatgaaaacaggagactaaaattaattatgcctgctaggttttatcccaatgttactaaatatttgcccttagataaagggatcaaaccttattatccagagcatgtagttaatcattacttccagacgagacattatttacacactctttggaaggcgggtatcttatataaaagagagtccacacgtagcgcctcattttgcgggtcaccatattcttgggaacaagatctacagcatgggaggttggtcttccaaacctcgaaaaggcatggggacaaatctttctgtccccaatcccctgggattcttccccgatcatcagttggaccctgcattcaaagccaactcagaaaatccagattgggacctcaacccgcacaaggacaactggccggacgccaacaaggtgggagtgggagcattcgggccagggttcacccctccccatgggggactgttggggtggagccctcaggctcagggcatactcacaactgtgccagcagctcctcctcctgcctccaccaatcggcagtcaggaaggcagcctactcccttatctccacctctaagggacactcatcctcaggccatgcagtggaactccaccactttccaccaaactcttcaagatcccagagtcagggccctgtactttcctgctggtggctccagttcaggaacagtgagccctgctcagaatactgtctctgccatatcgtcaatcttatcgaagactggggaccctgtaccgaacatggagaacatcgcatcaggactcctaggacccctgctcgtgttacaggcggggtttttcttgttgacaaaaatcctcacaataccacagagtctagactcgtggtggacttctctcaattttctagggggaacacccgtgtgtcttggccaaaattcgcagtcccaaatctccagtcactcaccaacctgttgtcctccaatttgtcctggttatcgctggatgtgtctgcggcgttttatcatcttcctctgcatcctgctgctatgcctcatcttcttgttggttcttctggactatcaaggtatgttgcccgtttgtcctctaattccaggatcatcaacaaccagcaccggaccatgcaaaacctgcacaactcctgctcaaggaacctctatgtttccctcatgttgctgtacaaaacctacggacggaaactgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttatgccgctgttaccaattttcttttgtctttgggtatacatttaaaccctcacaaaacaaaaagatggggatattcccttaacttcatgggatatgtaattgggagttggggcacattgccacaggaacatattgtacaaaaaatcaaaatgtgttttaggaaacttcctgtaaacaggcctattgattggaaagtatgtcaacgaattgtgggtcttttggggtttgccgcccctttcacgcaatgtggatatcctgctttaatgcctttatatgcatgtatacaagcaaaacaggcttttactttctcgccaacttacaaggcctttctaagtaaacagtatctgaacctttaccccgttgctcggcaacggcctggtctgtgccaagtgtttgctgacgcaacccccactggttggggcttggccataggccatcagcgcatgcgtggaacctttgtgtctcctctgccgatccatactgcggaactcctagccgcttgttttgctcgcagcaggtctggggcaaaactcatcgggactgacaattctgtcgtgctctcccgcaagtatacatcatttccatggctgctaggctgtgctgccaactggatcctgcgcgggacgtcctttgtttacgtcccgtcggcgctgaatcccgcggacgacccctcccggggccgcttggggctctaccgcccgcttctccgcctgttgtaccgaccgaccacggggcgcacctctctttacgcggactccccgtctgtgccttctcatctgccggaccgtgtgcacttcgcttcacctctgcacgtcgcatggagaccaccgtga

SEQ ID NO: 2 - DNA, 2107 bp, Hepatitis B virus Type C
caaaactaggcattatttacatactctgtggaaggctggcattctatataagagagaaactacacgcagcgcctcattttgtgggtcaccatattcttgggaacaagagctacagcatgggaggttggtcttccaaacctcgacaaggcatggggacgaatctttctgttcccaatcctctgggattctttcccgatcaccagttggaccctgcgttcggagccaactcaaacaatccagattgggacttcaaccccaacaaggatcactggccagaggcaaatcaggtaggagcgggagcattcgggccagggttcaccccaccacacggcggtcttttggggtggagccctcaggctcagggcatattgacaacagtgccagcagcgcctcctcctgcctccaccaatcggcagtcaggaagacagcctactcccatctctccacctctaagagacagtcatcctcaggccatgcagtggaactccacaacattccaccaagctctgctagatcccagagtgaggggcctatactttcctgctggtggctccagttccggaacagtaaaccctgttccgactactgcctcacccatatcgtcaatcttctcgaggactggggaccctgcaccgaacatggagagcacaacatcaggattcctaggacccctgctcgtgttacaggcggggtttttcttgttgacaagaatcctcacaataccacagagtctagactcgtggtggacttctctcaattttctagggggagcacccacgtgtcctggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaactaccagcacgggaccatgcaagacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttgaaccctaataaaaccaaacgttggggctactcccttaacttcatgggatatgtaattggaagttggggtactttaccacaggaacatattgtactaaaaatcaagcaatgttttcgaaaactgcctgtaaatagacctattgattggaaagtatgtcaaagaattgtgggtcttttgggctttgctgccccttttacacaatgtggctatcctgccttaatgcctttatatgcatgtatacaatctaagcaggctttcactttctcgccaacttacaaggcctttctgtgtaaacaatatctgaacctttaccccgttgcccggcaacggtcaggtctctgccaagtgtttgctgacgcaacccccactggatggggcttggccataggccatcggcgcatgcgtggaacctttgtggctcctctgccgatccatactgcggaactcctagcagcttgttttgctcgcagccggtctggagcaaaacttatcggcaccgacaactctgttgtcctctctcggaaatacacctcctttccatggctgctagggtgtgctgccaactggatcctgcgcgggacgtcctttgtctacgtcccgtcggcgctgaatcccgcggacgacccgtctcggggccgtttgggactctaccgtccccttcttcgtctgccgttccggccgaccacggggcgcacctctctttacgcggtctccccgtctgtgccttctcatctgccggaccgtgtgcacttcgcttcacctctgcacgtcgcatggagaccaccgtga

SEQ ID NO: 3 - DNA, 41 bp, Hepatitis B virus
cgaaccactgaacaaatggcctttcgcaaaatacctatggg

SEQ ID NO: 4 - DNA, 39 bp, Hepatitis B virus
ctttcccccactgtctggctgttgtacagacttggcccc

SEQ ID NO: 5 - DNA, 41 bp, Hepatitis B virus
cgaaccactgaacaaatggcctttcgcaagattcctatggg

SEQ ID NO: 6 - DNA, 40 bp, Hepatitis B virus
ctttcccccactgtttggcttgttgtacagacttggcccc

SEQ ID NO: 7 - DNA, 40 bp, Hepatitis B virus
tgttgtacagacttggccccctttcccccactgtctggct

SEQ ID NO: 8 - DNA, 42 bp, Hepatitis B virus
ccctttatgccgctgttaccccccatctttttgttttgtgag

SEQ ID NO: 9 - DNA, 40 bp, Hepatitis B virus
tgttgtacagacttggccccctttcccccactgtttggct

SEQ ID NO: 10 - DNA, 42 bp, Hepatitis B virus
ccctttttacctctattaccccccaacgtttggttttattag

SEQ ID NO: 11 - DNA, 400 bp, Hepatitis B virus
tcctgctcaaggaacctctatgtttccctcatgttgctgtacaaaacctacggacggaaactgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttatgccgctgttaccaattttcttttgtctttgggtatacatttaaaccctcacaaaacaaaaagatggggatattcccttaacttcatgggatatgtaattgggagttggggcacattgccacaggaacatattgtacaaa

SEQ ID NO: 12 - DNA, 399 bp, Hepatitis B virus
tcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttgaaccctaataaaaccaaacgttggggctactcccttaacttcatgggatatgtaattggaagttggggtactttaccacaggaacatattgtacta

SEQ ID NO: 13 - DNA, 400 bp, Hepatitis B virus
catcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttatgccgctgttaccaattttcttttgtctttgggtatacatttaaaccctcacaaaacaaaaagatggggatattcccttaacttcatgggatatgtaattgggagttggggcacattgccacaggaacatattgtacaaaaaatcaaaatgtgttttaggaaacttcctgtaaacaggcctattgattggaaagtatgtcaacgaattgtgggtcttttg

SEQ ID NO: 14 - DNA, 399 bp, Hepatitis B virus
catcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttgaaccctaataaaaccaaacgttggggctactcccttaacttcatgggatatgtaattggaagttggggtactttaccacaggaacatattgtactaaaaatcaagcaatgttttcgaaaactgcctgtaaatagacctattgattggaaagtatgtcaaagaattgtgggtctttt

SEQ ID NO: 15 - DNA, 48 bp, Hepatitis B virus
taagggaatatccccatctttttgtgctgttaccaattttcttttgtc

SEQ ID NO: 16 - DNA, 38 bp, Hepatitis B virus
gcacattgccacaggaacatggcctgtttacaggaagt

SEQ ID NO: 17 - DNA, 42 bp, Hepatitis B virus
taagggagtagccccaacgtaccaattttcttttgtctttgg

SEQ ID NO: 18 - DNA, 45 bp, Hepatitis B virus
ggatatgtaattggaagttggggtataggtctatttacaggcagt

SEQ ID NO: 19 - DNA, 47 bp, Hepatitis B virus
acatcatccatataactgaaagccagtttactagtgccatttgttca

SEQ ID NO: 20 - DNA, 47 bp, Hepatitis B virus
ccgctgttaccaattttcttttgtcccaattacatatcccatgaagt

SEQ ID NO: 21 - DNA, 43 bp, Hepatitis B virus
acatcatccatataactgaaagccatgccatttgttcagtggt

SEQ ID NO: 22 - DNA, 43 bp, Hepatitis B virus
acatcttgagtccctttttacctctgttaagggagtagcccca

SEQ ID NO: 23 - DNA, 43 bp, Hepatitis B virus
tgatgggatgggaatacaggtgaacctctatgtttccctcatg

SEQ ID NO: 24 - DNA, 40 bp, Hepatitis B virus
cttgggctttcgcaaaatacctaaagccctacgaaccact

SEQ ID NO: 25 - DNA, 43 bp, Hepatitis B virus
tgatgggatgggaatacaagtgccacctctatgtttccctctt

SEQ ID NO: 26 - DNA, 39 bp, Hepatitis B virus
tgggctttcgcaagattcctcgaaccactgaacaaatgg

SEQ ID NO: 27 - DNA, 20 bp, Hepatitis B virus
tgcacctgtattcccatccc

SEQ ID NO: 28 - DNA, 20 bp, Hepatitis B virus
acagcggcataaagggactc

SEQ ID NO: 29 - DNA, 20 bp, Hepatitis B virus
tgcacttgtattcccatccc

SEQ ID NO: 30 - DNA, 21 bp, Hepatitis B virus
aatagaggtaaaaagggactc

SEQ ID NO: 31 - DNA, 20 bp, Hepatitis B virus
gccatttgttcagtggttcg

SEQ ID NO: 32 - DNA, 21 bp, Hepatitis B virus
catatcccatgaagttaaggg

SEQ ID NO: 33 - DNA, 19 bp, Hepatitis B virus
tcttgagtccctttatgcc

SEQ ID NO: 34 - DNA, 18 bp, Hepatitis B virus
acccacaattcgttgaca

SEQ ID NO: 35 - DNA, 21 bp, Hepatitis B virus
caagtctgtacaacatcttga

SEQ ID NO: 36 - DNA, 22 bp, Hepatitis B virus
tctctgacatactttccaatca

SEQ ID NO: 37 - DNA, 19 bp, Hepatitis B virus
cctcagtccgtttctcttg

SEQ ID NO: 38 - DNA, 18 bp, Hepatitis B virus
tgttcctgtggcaatgtg

SEQ ID NO: 39 - DNA, 18 bp, Hepatitis B virus
tcagtccgtttctcctgg

SEQ ID NO: 40 - DNA, 22 bp, Hepatitis B virus
acttccaattacatatcccatg

SEQ ID NO: 41 - DNA, 18 bp, Hepatitis B virus
cacaactcctgctcaagg

SEQ ID NO: 42 - DNA, 18 bp, Hepatitis B virus
ctgaaagccagacagtgg

SEQ ID NO: 43 - DNA, 18 bp, Hepatitis B virus
cacgattcctgctcaagg

SEQ ID NO: 44 - DNA, 22 bp, Hepatitis B virus
atataactgaaagccaaacagt

SEQ ID NO: 45 - DNA, 17 bp, Hepatitis B virus
cggactgaggcccactc

SEQ ID NO: 46 - DNA, 20 bp, Hepatitis B virus
agaaacggactgaggcccac

SEQ ID NO: 47 - DNA, 20 bp, Hepatitis B virus
cagttatatggatgatgtgg

SEQ ID NO: 48 - DNA, 22 bp, Hepatitis B virus
ccaattttcttttgtctttggg

SEQ ID NO: 49 - DNA, 20 bp, Hepatitis B virus
ccacatcatccatataactg

SEQ ID NO: 50 - DNA, 15 bp, Hepatitis B virus
agttatatggatgat

SEQ ID NO: 51 - DNA, 15 bp, Hepatitis B virus
agttatatcgatgat

SEQ ID NO: 52 - DNA, 15 bp, Hepatitis B virus
agttatattgatgat

SEQ ID NO: 53 - DNA, 15 bp, Hepatitis B virus
agttatatagatgat

SEQ ID NO: 54 - DNA, 15 bp, Hepatitis B virus
agttatgtggatgat

SEQ ID NO: 55 - DNA, 15 bp, Hepatitis B virus
agttatgtcgatgat

SEQ ID NO: 56 - DNA, 15 bp, Hepatitis B virus
agttatgttgatgat

SEQ ID NO: 57 - DNA, 15 bp, Hepatitis B virus
agttatgtagatgat

SEQ ID NO: 58 - DNA, 15 bp, Hepatitis B virus
catttaaaccctcac

SEQ ID NO: 59 - DNA, 15 bp, Hepatitis B virus
catttaaatcctcac

SEQ ID NO: 60 - DNA, 15 bp, Hepatitis B virus
catttaactcctcac

SEQ ID NO: 61 - DNA, 15 bp, Hepatitis B virus
catttaacccctcac

SEQ ID NO: 62 - DNA, 15 bp, Hepatitis B virus
catttaacgcctcac

SEQ ID NO: 63 - DNA, 15 bp, Hepatitis B virus
catttaacacctcac

SEQ ID NO: 64 - DNA, 15 bp, Hepatitis B virus
catttgaaccctaat

SEQ ID NO: 65 - DNA, 15 bp, Hepatitis B virus
catttgaatcctaat

SEQ ID NO: 66 - DNA, 15 bp, Hepatitis B virus
catttgactcctaat

SEQ ID NO: 67 - DNA, 15 bp, Hepatitis B virus
catttgacccctaat

SEQ ID NO: 68 - DNA, 15 bp, Hepatitis B virus
catttgacgcctaat

SEQ ID NO: 69 - DNA, 15 bp, Hepatitis B virus
catttgacacctaat

SEQ ID NO: 70 - DNA, 15 bp, Hepatitis B virus
catttaaaccctaat

SEQ ID NO: 71 - DNA, 15 bp, Hepatitis B virus
catttaaatcctaat

SEQ ID NO: 72 - DNA, 15 bp, Hepatitis B virus
catttaactcctaat

SEQ ID NO: 73 - DNA, 15 bp, Hepatitis B virus
catttaacccctaat

SEQ ID NO: 74 - DNA, 15 bp, Hepatitis B virus
catttaacgcctaat

SEQ ID NO: 75 - DNA, 15 bp, Hepatitis B virus
catttaacacctaat

SEQ ID NO: 76 - DNA, 15 bp, Hepatitis B virus
ctcttggctcagttt

SEQ ID NO: 77 - DNA, 15 bp, Hepatitis B virus
ctcttggcccagttt

SEQ ID NO: 78 - DNA, 15 bp, Hepatitis B virus
ctcttggcacagttt

SEQ ID NO: 79 - DNA, 15 bp, Hepatitis B virus
ctcttggcgcagttt

SEQ ID NO: 80 - DNA, 15 bp, Hepatitis B virus
ctcttggttcagttt

SEQ ID NO: 81 - DNA, 15 bp, Hepatitis B virus
ctcttggtccagttt

SEQ ID NO: 82 - DNA, 15 bp, Hepatitis B virus
ctcttggtacagttt

SEQ ID NO: 83 - DNA, 15 bp, Hepatitis B virus
ctcttggtgcagttt

SEQ ID NO: 84 - DNA, 15 bp, Hepatitis B virus
ctcctggctcagttt

SEQ ID NO: 85 - DNA, 15 bp, Hepatitis B virus
ctcctggcccagttt

SEQ ID NO: 86 - DNA, 15 bp, Hepatitis B virus
ctcctggcacagttt

SEQ ID NO: 87 - DNA, 15 bp, Hepatitis B virus
ctcctggcgcagttt

SEQ ID NO: 88 - DNA, 15 bp, Hepatitis B virus
ctcctggttcagttt

SEQ ID NO: 89 - DNA, 15 bp, Hepatitis B virus
ctcctggtccagttt

SEQ ID NO: 90 - DNA, 15 bp, Hepatitis B virus
ctcctggtacagttt

SEQ ID NO: 91 - DNA, 15 bp, Hepatitis B virus
ctcctggtgcagttt

SEQ ID NO: 92 - DNA, 18 bp, Hepatitis B virus
cagttatatggatgatgt

SEQ ID NO: 93 - DNA, 19 bp, Hepatitis B virus
tcagttatatggatgatgt

SEQ ID NO: 94 - DNA, 20 bp, Hepatitis B virus
tcagttatatggatgatgtg

SEQ ID NO: 95 - DNA, 18 bp, Hepatitis B virus
cagttatatcgatgatgt

SEQ ID NO: 96 - DNA, 19 bp, Hepatitis B virus
tcagttatatcgatgatgt

SEQ ID NO: 97 - DNA, 20 bp, Hepatitis B virus
tcagttatatcgatgatgtg

SEQ ID NO: 98 - DNA, 19 bp, Hepatitis B virus
cagttatattgatgatgtg

SEQ ID NO: 99 - DNA, 20 bp, Hepatitis B virus
tcagttatattgatgatgtg

SEQ ID NO: 100 - DNA, 21 bp, Hepatitis B virus
ttcagttatattgatgatgtg

SEQ ID NO: 101 - DNA, 22 bp, Hepatitis B virus
ttcagttatattgatgatgtgg

SEQ ID NO: 102 - DNA, 23 bp, Hepatitis B virus
tttcagttatattgatgatgtgg

SEQ ID NO: 103 - DNA, 19 bp, Hepatitis B virus
cagttatatagatgatgtg

SEQ ID NO: 104 - DNA, 20 bp, Hepatitis B virus
tcagttatatagatgatgtg

SEQ ID NO: 105 - DNA, 21 bp, Hepatitis B virus
ttcagttatatagatgatgtg

SEQ ID NO: 106 - DNA, 22 bp, Hepatitis B virus
ttcagttatatagatgatgtgg

SEQ ID NO: 107 - DNA, 17 bp, Hepatitis B virus
tcagttatgtggatgat

SEQ ID NO: 108 - DNA, 18 bp, Hepatitis B virus
ttcagttatgtggatgat

SEQ ID NO: 109 - DNA, 19 bp, Hepatitis B virus
ttcagttatgtggatgatg

SEQ ID NO: 110 - DNA, 20 bp, Hepatitis B virus
tttcagttatgtggatgatg

SEQ ID NO: 111 - DNA, 21 bp, Hepatitis B virus
ctttcagttatgtggatgatg

SEQ ID NO: 112 - DNA, 17 bp, Hepatitis B virus
tcagttatgtcgatgat

SEQ ID NO: 113 - DNA, 18 bp, Hepatitis B virus
ttcagttatgtcgatgat

SEQ ID NO: 114 - DNA, 20 bp, Hepatitis B virus
tttcagttatgtcgatgatg

SEQ ID NO: 115 - DNA, 18 bp, Hepatitis B virus
tcagttatgttgatgatg

SEQ ID NO: 116 - DNA, 19 bp, Hepatitis B virus
ttcagttatgttgatgatg

SEQ ID NO: 117 - DNA, 21 bp, Hepatitis B virus
tttcagttatgttgatgatgt

SEQ ID NO: 118 - DNA, 19 bp, Hepatitis B virus
ttcagttatgtagatgatg

SEQ ID NO: 119 - DNA, 20 bp, Hepatitis B virus
tttcagttatgtagatgatg

SEQ ID NO: 120 - DNA, 22 bp, Hepatitis B virus
ctttcagttatgtagatgatgt

SEQ ID NO: 121 - DNA, 49 bp, Hepatitis B virus
ttaccaattttcttttgtctttgggattacatatcccatgaagttaagg

SEQ ID NO: 122 - DNA, 49 bp, Hepatitis B virus; identical to SEQ ID NO: 121

SEQ ID NO: 123 - DNA, 17 bp, Hepatitis B virus
atgtgccccaactccca

SEQ ID NO: 124 - DNA, 20 bp, Hepatitis B virus
gtaaagtaccccaacttcca

SEQ ID NO: 125 - DNA, 20 bp, Hepatitis B virus
caaaaagatggggatattcc

SEQ ID NO: 126 - DNA, 17 bp, Hepatitis B virus
ccaaacgttggggctac

SEQ ID NO: 127-131 - skipped (5 entries)

SEQ ID NO: 132 - DNA, 15 bp, Hepatitis B virus
ctcatggctcagttt

SEQ ID NO: 133 - DNA, 15 bp, Hepatitis B virus
ctcatggcccagttt

SEQ ID NO: 134 - DNA, 15 bp, Hepatitis B virus
ctcatggcacagttt

SEQ ID NO: 135 - DNA, 15 bp, Hepatitis B virus
ctcatggcgcagttt

SEQ ID NO: 136 - DNA, 15 bp, Hepatitis B virus
ctcatggttcagttt

SEQ ID NO: 137-146 - skipped (10 entries)

SEQ ID NO: 147 - DNA, 17 bp, Hepatitis B virus
tacatttaaaccctcac

SEQ ID NO: 148 - DNA, 18 bp, Hepatitis B virus
atacatttaaaccctcac

SEQ ID NO: 149 - DNA, 20 bp, Hepatitis B virus
atacatttaaatcctcacaa

SEQ ID NO: 150 - DNA, 22 bp, Hepatitis B virus
tatacatttaaatcctcacaaa

SEQ ID NO: 151 - DNA, 17 bp, Hepatitis B virus
atacatttaacccctca

SEQ ID NO: 152 - DNA, 18 bp, Hepatitis B virus
atacatttaacccctcac

SEQ ID NO: 153 - DNA, 19 bp, Hepatitis B virus
atacatttaacccctcaca

SEQ ID NO: 154 - DNA, 19 bp, Hepatitis B virus
acatttgaaccctaataaa

SEQ ID NO: 155 - DNA, 20 bp, Hepatitis B virus
acatttgaaccctaataaaa

SEQ ID NO: 156 - DNA, 22 bp, Hepatitis B virus
atacatttgaaccctaataaaa

SEQ ID NO: 157 - DNA, 23 bp, Hepatitis B virus
atacatttgaatcctaataaaac

SEQ ID NO: 158 - DNA, 21 bp, Hepatitis B virus
tacatttgacccctaataaaa

SEQ ID NO: 159 - DNA, 22 bp, Hepatitis B virus
tacatttgacccctaataaaac

SEQ ID NO: 160 - DNA, 21 bp, Hepatitis B virus
atacatttaaaccctaataaa

SEQ ID NO: 161 - DNA, 22 bp, Hepatitis B virus
atacatttaaaccctaataaaa

SEQ ID NO: 162 - DNA, 23 bp, Hepatitis B virus
atacatttaaaccctaataaaac

SEQ ID NO: 163 - DNA, 23 bp, Hepatitis B virus
tatacatttaaaccctaataaaa

SEQ ID NO: 164 - DNA, 24 bp, Hepatitis B virus
tatacatttaaatcctaataaaac

SEQ ID NO: 165 - DNA, 25 bp, Hepatitis B virus
gtatacatttaaatcctaataaaac

SEQ ID NO: 166 - DNA, 23 bp, Hepatitis B virus
tatacatttaacccctaataaaa

SEQ ID NO: 167 - DNA, 24 bp, Hepatitis B virus
tatacatttaacccctaataaaac

SEQ ID NO: 168 - DNA, 16 bp, Hepatitis B virus
taaactgagccaagag

SEQ ID NO: 169 - DNA, 17 bp, Hepatitis B virus
gtaaactgagccaagag

SEQ ID NO: 170 - DNA, 18 bp, Hepatitis B virus
gtaaactgagccaagaga

SEQ ID NO: 171 - DNA, 19 bp, Hepatitis B virus
agtaaactgggccaagaga

SEQ ID NO: 172 - DNA, 21 bp, Hepatitis B virus
agtaaactgggccaagagaaa

SEQ ID NO: 173 - DNA, 18 bp, Hepatitis B virus
agtaaactgtgccaagag

SEQ ID NO: 174 - DNA, 16 bp, Hepatitis B virus
gtaaactgcgccaaga

SEQ ID NO: 175 - DNA, 18 bp, Hepatitis B virus
taaactgaaccaagagaa

SEQ ID NO: 176 - DNA, 19 bp, Hepatitis B virus
gtaaactgaaccaagagaa

SEQ ID NO: 177 - DNA, 20 bp, Hepatitis B virus
gtaaactgaaccaagagaaa

SEQ ID NO: 178 - DNA, 16 bp, Hepatitis B virus
taaactgagccaggag

SEQ ID NO: 179 - DNA, 17 bp, Hepatitis B virus
taaactgagccaggaga

SEQ ID NO: 180 - DNA, 18 bp, Hepatitis B virus
gtaaactgagccaggaga

SEQ ID NO: 181 - DNA, 18 bp, Hepatitis B virus
gtaaactgggccaggaga

SEQ ID NO: 182 - DNA, 20 bp, Hepatitis B virus
agtaaactgggccaggagaa

SEQ ID NO: 183 - DNA, 18 bp, Hepatitis B virus
gtaaactgtgccaggaga

SEQ ID NO: 184 - DNA, 17 bp, Hepatitis B virus
gtaaactgcgccaggag

SEQ ID NO: 185 - DNA, 17 bp, Hepatitis B virus
taaactgaaccaggaga

SEQ ID NO: 186 - DNA, 18 bp, Hepatitis B virus
gtaaactgaaccaggaga

SEQ ID NO: 187 - DNA, 19 bp, Hepatitis B virus
gtaaactgaaccaggagaa

SEQ ID NO: 188 - DNA, 16 bp, Hepatitis B virus
gtaaactgagccatga

SEQ ID NO: 189 - DNA, 17 bp, Hepatitis B virus
gtaaactgagccatgag

SEQ ID NO: 190 - DNA, 18 bp, Hepatitis B virus
gtaaactgggccatgaga

SEQ ID NO: 191 - DNA, 21 bp, Hepatitis B virus
agtaaactgggccatgagaaa

SEQ ID NO: 192 - DNA, 18 bp, Hepatitis B virus
agtaaactgtgccatgag

SEQ ID NO: 193 - DNA, 16 bp, Hepatitis B virus
gtaaactgcgccatga

SEQ ID NO: 194 - DNA, 17 bp, Hepatitis B virus
gtaaactgaaccatgag

SEQ ID NO: 195 - DNA, 18 bp, Hepatitis B virus
gtaaactgaaccatgaga

SEQ ID NO: 196 - DNA, 19 bp, Hepatitis B virus
gtaaactgaaccatgagaa

SEQ ID NO: 197 - DNA, 480 bp, Hepatitis B virus
cacaactcctgctcaaggaacctctatgtttccctcatgttgctgtacaaaacctacggacggaaactgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggtattgggggccaagtctgtacaacatcttgagtccctttatgccgctgttaccaattttcttttgtctttgggtatacatttaaaccctcacaaaacaaaaagatggggatattcccttaacttcatgggatatgtaattgggagttggggcacattgccacaggaacatattgtacaaaaaatcaaactttgttttaggaaacttcctgtaaacaggcctattgattggaaagtttgtcaacgaattgtgggt

SEQ ID NO: 198 - DNA, 471 bp, Hepatitis B virus
cacgattcctgctcaaggcacctctatgtttccctcttgttgctgtacaaaaccttcggatggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttgaaccctaataaaaccaaacgttggggctactcccttaacttcatgggatatgtaattggaagttggggtactttaccgcaagaccatattgtactaaaactcaagcaatgttttcgaaaactgcctgtaaatagacctattgattggaaagtatgtcagaga

SEQ ID NO: 199 - DNA, 294 bp, Hepatitis B virus
tgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttatgccgctgttaccaattttcttttgtctttgggtatacatttaaaccctcacaaaacaaaaagatggggatattcccttaacttcatgggatatg

SEQ ID NO: 200 - DNA, 1000 bp, Hepatitis B virus
acagagtctagactcgtggtggacttctctcaattttctagggggaacacccgtgtgtcttggccaaaattcgcagtcccaaatctccagtcactcaccaacctgttgtcctccaatttgtcctggttatcgctggatgtgtctgcggcgttttatcatcttcctctgcatcctgctgctatgcctcatcttcttgttggttcttctggactatcaaggtatgttgcccgtttgtcctctaattccaggatcatcaacaaccagcaccggaccatgcaaaacctgcacaactcctgctcaaggaacctctatgtttccctcatgttgctgtacaaaacctacggacggaaactgcacctgtattcccatcccatcatcttgggctttcgcaaaatacctatgggagtgggcctcagtccgtttctcttggctcagtttactagtgccatttgttcagtggttcgtagggctttcccccactgtctggctttcagttatatggatgatgtggttttgggggccaagtctgtacaacatcttgagtccctttatgccgctgttaccaattttcttttgtctttgggtatacatttaaaccctcacaaaacaaaaagatggggatattcccttaacttcatgggatatgtaattgggagttggggcacattgccacaggaacatattgtacaaaaaatcaaaatgtgttttaggaaacttcctgtaaacaggcctattgattggaaagtatgtcaacgaattgtgggtcttttggggtttgccgcccctttcacgcaatgtggatatcctgctttaatgcctttatatgcatgtatacaagcaaaacaggcttttactttctcgccaacttacaaggcctttctaagtaaacagtatctgaacctttaccccgttgctcggcaacggcctggtctgtgccaagtgtttgctgacgcaacccccactggttggggcttggccataggccatcagcgcatgcgtg

SEQ ID NO: 201 - DNA, 1000 bp, Hepatitis B virus
acagagtctagactcgtggtggacttctctcaattttctagggggagcacccacgtgtcctggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaactaccagcacgggaccatgcaagacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcctggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttgaaccctaataaaaccaaacgttggggctactcccttaacttcatgggatatgtaattggaagttggggtactttaccacaggaacatattgtactaaaaatcaagcaatgttttcgaaaactgcctgtaaatagacctattgattggaaagtatgtcaaagaattgtgggtcttttgggctttgctgccccttttacacaatgtggctatcctgccttaatgcctttatatgcatgtatacaatctaagcaggctttcactttctcgccaacttacaaggcctttctgtgtaaacaatatctgaacctttaccccgttgcccggcaacggtcaggtctctgccaagtgtttgctgacgcaacccccactggatggggcttggccataggccatcggcgcatgcgtg

SEQ ID NO: 202 - DNA, 1000 bp, Hepatitis B virus; identical to SEQ ID NO: 200

SEQ ID NO: 203 - DNA, 1000 bp, Hepatitis B virus; identical to SEQ ID NO: 201

SEQ ID NO: 204 - DNA, 1000 bp, Hepatitis B virus
acagagtctagactcgtggtggacttctctcaattttctagggggagcacccacgtgtcctggccaaaattcgcagtccccaacctccaatcactcaccaacctcttgtcctccaatttgtcctggctatcgctggatgtgtctgcggcgttttatcatattcctcttcatcctgctgctatgcctcatcttcttgttggttcttctggactaccaaggtatgttgcccgtttgtcctctacttccaggaacatcaactaccagcacgggaccatgcaagacctgcacgattcctgctcaaggaacctctatgtttccctcttgttgctgtacaaaaccttcggacggaaactgcacttgtattcccatcccatcatcctgggctttcgcaagattcctatgggagtgggcctcagtccgtttctcatggctcagtttactagtgccatttgttcagtggttcgtagggctttcccccactgtttggctttcagttatatggatgatgtggtattgggggccaagtctgtacaacatcttgagtccctttttacctctattaccaattttcttttgtctttgggtatacatttgaaccctaataaaaccaaacgttggggctactcccttaacttcatgggatatgtaattggaagttggggtactttaccacaggaacatattgtactaaaaatcaagcaatgttttcgaaaactgcctgtaaatagacctattgattggaaagtatgtcaaagaattgtgggtcttttgggctttgctgccccttttacacaatgtggctatcctgccttaatgcctttatatgcatgtatacaatctaagcaggctttcactttctcgccaacttacaaggcctttctgtgtaaacaatatctgaacctttaccccgttgcccggcaacggtcaggtctctgccaagtgtttgctgacgcaacccccactggatggggcttggccataggccatcggcgcatgcgtg

(19) United States
(12) Patent Application Publication
(10) Pub. No.: US 2010/0248210 A1
(43) Pub. Date: Sep. 30, 2010

(54) Nucleic acid primer set for detection of drug-resistant strain of hepatitis B virus, assay kit, and method of detecting drug-resistant strain of hepatitis B virus

(76) Inventors: Masayoshi Takahashi, Tokyo (JP); Michie Hashimoto, Tokyo (JP); Keiko Ito, Kawasaki-shi (JP); Keiko Kizu, Fuchu-shi (JP); Shunji Mishiro, Tokyo (JP); Kazuaki Takahashi, Machida-shi (JP); Kazunori Miyazaki, Yokohama-shi (JP); Koji Hashimoto, Atsugi-shi (JP); Nobuhiro Gemma, Yokohama-shi (JP)

Correspondence Address: OBLON, SPIVAK, MCCLELLAND MAIER & NEUSTADT, L.L.P., 1940 DUKE STREET, ALEXANDRIA, VA, 22314, US

(21) Appl. No.: 12/382,733

(22) Filed: Mar. 23, 2009

Related U.S. Application Data
(63) Continuation of application No. PCT/JP08/70266, filed on Oct. 30, 2008.

(30) Foreign Application Priority Data
Oct. 30, 2007 (JP) 2007-282279
Feb. 14, 2008 (JP) 2008-033725
Oct. 27, 2008 (JP) 2008-275200

(51) Int. Cl. C12Q 1/70

(52) U.S. Cl. 435/5

(57) ABSTRACT

The invention provides a method of detecting a drug-resistant strain of hepatitis B virus, including amplifying a hepatitis B virus nucleic acid in a sample solution by LAMP with a primer set to yield an amplification product, and hybridizing the amplification product with a probe containing a polynucleotide derived from a drug-resistant strain and/or a probe containing a polynucleotide derived from a drug-nonresistant strain, to detect a drug-resistant strain of hepatitis B virus.

### CROSS-REFERENCE TO RELATED APPLICATIONS

This application is based upon and claims the benefit of priority from prior Japanese Patent Applications No. 2007-282279, filed Oct. 30, 2007; No. 2008-033725, filed Feb. 14, 2008; and No. 2008-275200, filed Oct. 27, 2008, the entire contents of all of which are incorporated herein by reference.

### BACKGROUND OF THE INVENTION

1. Field of the Invention

The present invention relates to a nucleic acid primer set used for detecting a nucleotide sequence encoding an amino acid sequence specific for drug-resistant hepatitis B virus, an assay kit, and a method of detecting a drug-resistant strain of hepatitis B virus.

2. Description of the Related Art

As a method for treating HBV patients, there is a method of administering a chemical that inhibits HBV multiplication, such as a reverse transcriptase inhibitor. Such a chemical inhibits HBV multiplication both by binding to a site to which dCTP normally binds during DNA replication, thereby exhibiting an action of competitively inhibiting the work of a reverse transcriptase in incorporating dCTP and by being incorporated into a DNA (−) chain during replication, thereby exhibiting an action of preventing the elongation of the DNA chain.

It is however known that when lamivudine for example is administered for a long time, there may appear a mutant virus having a mutation in a certain amino acid region (for example, YMDD region), and this mutant virus is resistant to lamivudine, thus causing re-increase in the amount of the virus (“Kanzo” (Liver), Vol. 47, No. 11, 499-502 (2006)).

Accordingly, when the appearance of a drug-resistant strain is indicated by monitoring, urgent countermeasures such as change of a drug etc. are required. Detection of a drug-resistant strain is carried out by amplifying DNA from hepatitis B virus (referred to hereinafter as HBV) by PCR and then reading, by sequence analysis, a nucleotide sequence encoding the above amino acid mutation (Int. J. Med. Sci. 2005 2(1)). However, there is desire for a method of detecting a drug-resistant strain in an easier manner.

### BRIEF SUMMARY OF THE INVENTION

In view of the problem described above, the object of the present invention is to provide nucleic acid primer sets capable of detecting a drug-resistant strain of HBV easily and inexpensively in a short time, an assay kit, and a method of detecting a drug-resistant strain of hepatitis B virus.

To achieve the object, the present invention provides a method of detecting a drug-resistant or drug-nonresistant strain of hepatitis B virus, comprising:

amplifying a hepatitis B virus nucleic acid in a sample solution by LAMP with a primer set to yield an amplification product, and

hybridizing the amplification product with a probe containing a polynucleotide derived from a drug-resistant strain of hepatitis B virus and/or a probe containing a polynucleotide derived from a drug-nonresistant strain of hepatitis B virus, to detect a drug-resistant or drug-nonresistant strain of hepatitis B virus,

wherein the primer set comprises an FIP primer, an F3 primer, a BIP primer and a B3 primer, and at least one set is selected from the group consisting of:

a primer set 1 wherein the FIP primer is represented by SEQ ID NO: 3, the BIP primer is represented by SEQ ID NO: 4, the F3 primer is represented by SEQ ID NO: 27, and the B3 primer is represented by SEQ ID NO: 28, and

a primer set 2 wherein the FIP primer is represented by SEQ ID NO: 5, the BIP primer is represented by SEQ ID NO: 6, the F3 primer is represented by SEQ ID NO: 29, and the B3 primer is represented by SEQ ID NO: 30,

which are used for amplification of a nucleotide sequence region containing nucleotide sequences coding for amino acids at positions 181 and 204 in the polymerase region of hepatitis B virus;

a primer set 3 wherein the FIP primer is represented by SEQ ID NO: 7, the BIP primer is represented by SEQ ID NO: 8, the F3 primer is represented by SEQ ID NO: 31, and the B3 primer is represented by SEQ ID NO: 32,

a primer set 4 wherein the FIP primer is represented by SEQ ID NO: 9, the BIP primer is represented by SEQ ID NO: 10, the F3 primer is represented by SEQ ID NO: 31, and the B3 primer is represented by SEQ ID NO: 32,

a primer set 11 wherein the FIP primer is a polynucleotide represented by SEQ ID NO: 7, the BIP primer is represented by SEQ ID NO: 121, the F3 primer is represented by SEQ ID NO: 31, and the B3 primer is represented by SEQ ID NO: 123, and

a primer set 12 wherein the FIP primer is a polynucleotide represented by SEQ ID NO: 9, the BIP primer is represented by SEQ ID NO: 122, the F3 primer is represented by SEQ ID NO: 31, and the B3 primer is represented by SEQ ID NO: 124,

which are used for amplification of a nucleotide sequence region containing nucleotide sequences coding for amino acids at positions 204 and 236 in the polymerase region of hepatitis B virus;

a primer set 5 wherein the FIP primer is a polynucleotide represented by SEQ ID NO: 15, the BIP primer is represented by SEQ ID NO: 16, the F3 primer is represented by SEQ ID NO: 33, and the B3 primer is represented by SEQ ID NO: 34,

a primer set 6 wherein the FIP primer is a polynucleotide represented by SEQ ID NO: 17, the BIP primer is represented by SEQ ID NO: 18, the F3 primer is represented by SEQ ID NO: 35, and the B3 primer is represented by SEQ ID NO: 36,

a primer set 7 wherein the FIP primer is a polynucleotide represented by SEQ ID NO: 19, the BIP primer is represented by SEQ ID NO: 20, the F3 primer is represented by SEQ ID NO: 37, and the B3 primer is represented by SEQ ID NO: 38, and

a primer set 8 wherein the FIP primer is a polynucleotide represented by SEQ ID NO: 21, the BIP primer is represented by SEQ ID NO: 22, the F3 primer is represented by SEQ ID NO: 39, and the B3 primer is represented by SEQ ID NO: 40,

which are used for amplification of a nucleotide sequence region containing a nucleotide sequence coding for an amino acid at position 236 in the polymerase region of hepatitis B virus; and

a primer set 9 wherein the FIP primer is a polynucleotide represented by SEQ ID NO: 23, the BIP primer is represented by SEQ ID NO: 24, the F3 primer is represented by SEQ ID NO: 41, and the B3 primer is represented by SEQ ID NO: 42, and

a primer set 10 wherein the FIP primer is a polynucleotide represented by SEQ ID NO: 25, the BIP primer is represented by SEQ ID NO: 26, the F3 primer is represented by SEQ ID NO: 43, and the B3 primer is represented by SEQ ID NO: 44,

which are used for amplification of a nucleotide sequence region containing a nucleotide sequence coding for an amino acid at position 181 in the polymerase region of hepatitis B virus.

According to the present invention, a drug-resistant or drug-nonresistant strain of HBV can be detected easily, inexpensively and accurately in a short time.

### BRIEF DESCRIPTION OF THE SEVERAL VIEWS OF THE DRAWING

FIG. 1 is a schematic diagram of the LAMP method.

FIG. 2 is a schematic diagram showing intermediate products of the LAMP method and annealing positions of inner primers (FIP, BIP).

FIG. 3 is a schematic diagram showing the arrangement of loop primers.

FIG. 4 is a schematic diagram showing intermediate products of the LAMP method and annealing positions of loop primers (LFc, LBc).

FIG. 5 is a schematic view showing detection positions of an amplification product.

FIG. 6 is a plain schematic view of one embodiment of a probe-immobilized substrate.

FIG. 7 is a plain schematic view of one embodiment of a probe-immobilized substrate.

FIG. 8 shows accession numbers of HBV sequences registered in the databank.

FIG. 9 shows accession numbers of HBV sequences registered in the databank.

FIG. 10 shows a standard sequence of HBV, type B (SEQ ID NO: 1).

FIG. 11 shows a standard sequence of HBV, Type C (SEQ ID NO:2).

FIG. 12 shows nucleic acid primer sequences and relative positions of detection target sequences. Primer set 1 (SEQ ID NO: 1, nucleotides 1441-1840); Primer set 2 (SEQ ID NO: 2, nucleotides 1016-1414); Primer set 3 (SEQ ID NO: 1, nucleotides 1521-1920); Primer set 4 (SEQ ID NO: 2, nucleotides 1096-1494).

### DETAILED DESCRIPTION OF THE INVENTION

A drug-resistant strain of HBV is one type of mutant strain. The drug-resistant strain differs from a strain (wild-type strain) that is drug-nonresistant in amino acid at position 181, 204 or 236 in an amino acid sequence of the polymerase region of HBV. Such mutation is derived from a mutation in the gene of HBV, and the mutation site is due to the presence of a mutation in a nucleotide sequence encoding the amino acid sequence. Accordingly, the drug-resistant strain of HBV can be detected by identifying a nucleotide polymorphism based on a base mutation in the region, and consequently, it can be judged whether HBV from a subject such as a patient infected with HBV is drug-resistant or not.

A nucleotide polymorphism based on such a base mutation (hereinafter referred to as nucleotide polymorphism) has been detected mainly by PCR (polymerase chain reaction). In the PCR method, however, pretreatment such as nucleic acid extraction is cumbersome. Further, there is an inconvenience that complex temperature control by a thermal cycler or the like is essential and the reaction time requires 2 hours or more. There is a further problem that because the amplification product of the PCR method is a double strand, the complementary strand acts as a competitor for a probe in detection, to cause deterioration in detection sensitivity. It follows that for making the amplification product into a single strand, a method of decomposing or separating the complementary strand with an enzyme or magnetic beads is used, but in either case, there are problems such as troublesome operation and higher costs.

In the present invention, therefore, LAMP (loop-mediated isothermal amplification) is used in place of PCR, and the amplification product is hybridized with a nucleic acid probe, to detect the hybridization thereby detecting the nucleotide polymorphism.

The LAMP method is a technique of amplifying a nucleic acid at 60 to 65° C. under isothermal conditions. The LAMP method is advantageous over the PCR method in that a large amount of amplification products can be obtained in a short time. It is also reported that the LAMP method is hardly influenced by impurities in a sample. By using the LAMP method, a target nucleic acid can be easily amplified.

For example, a means including, but not limited to, nucleic acid probes can be used in measurement to detect the amplification product. The nucleic acid probes may be any probes that specifically detect a region amplified by the LAMP amplification primers in accordance with the present invention. The nucleic acid probes are those complementary to an amplification product of the wild type (that is, the non-resistant strain) and to an amplification product of the mutant type (that is, the resistant strain) respectively and having lower crossreactivity with one another, and the respective nucleic acid probes are used to hybridize with the respective amplification products, and the amplification products that were bound to the respective nucleic acid probes are detected. The amplification product bound to the wild-type nucleic acid probe and the amplification product bound to the mutant-type nucleic acid probe can be detected respectively to judge whether the HBV virus in the sample is a drug-resistant or not.

<Outline of the LAMP Method>

Hereinafter, the LAMP method is outlined. In this specification, a nucleic acid subjected to detection of nucleotide polymorphism is referred to as a sample nucleic acid. A nucleic acid containing a region encoding an amino acid at position 181, 204 or 236 in the polymerase region of HBV amplified by the LAMP amplification primers in accordance with the present invention is referred to as a target nucleic acid. A product obtained by the LAMP method is referred to as an amplification product. A solution containing HBV as the subject of amplification is referred to as a sample solution.

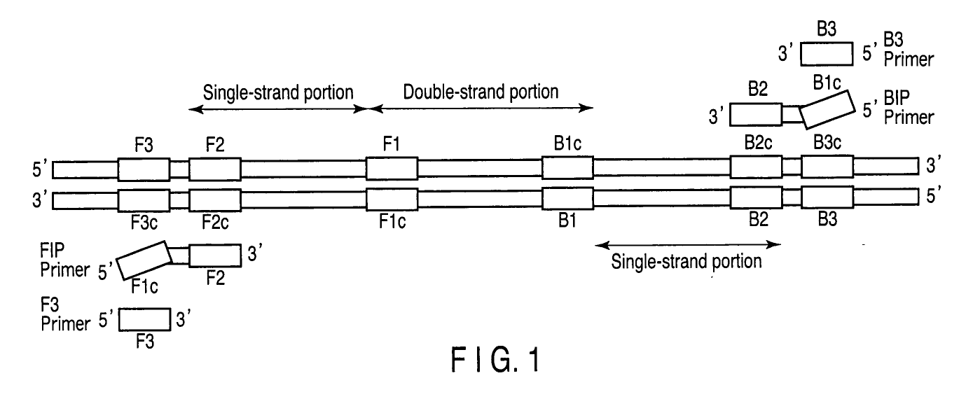

In the LAMP method, F3, F2 and F1 regions are placed in this order from the 5′-terminal side of the target nucleic acid, and B3c, B2c and B1c regions are placed in this order from the 3′-terminal side. Four kinds of primers as shown in FIG. 1 are used to amplify the target nucleic acid. F1c, F2c, F3c, B1, B2 and B3 regions refer respectively to regions, in a complementary strand, of F1, F2, F3, B1c, B2c and B3c regions.

The 4 kinds of primers used in amplification of the nucleic acid in the LAMP method are (1) FIP primer having, at its 3′-terminal side, the same sequence as the F2 region and having, at its 5′-terminal side, a complementary sequence to the F1 region; (2) F3 primer consisting of the same sequence as the F3 region; (3) BIP primer having, at its 3′-terminal side, a complementary sequence to the B2c region and having, at its 5′-terminal side, the same sequence as the B1c region; and (4) B3 primer consisting of a complementary sequence to the B3c region. Generally, the FIP primer and BIP primer are called inner primers, and the F3 primer and B3 primer are called outer primers.

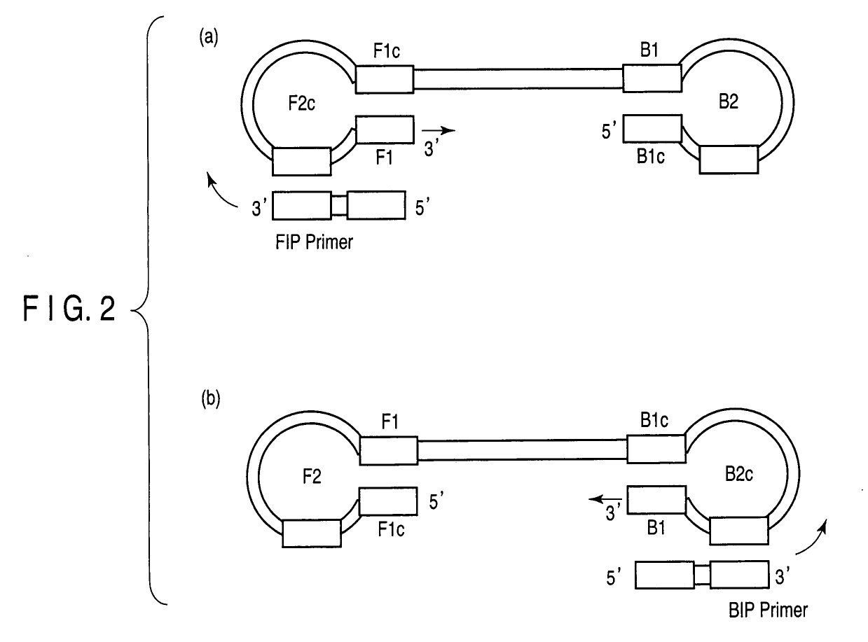

When the 4 kinds of primers are used in LAMP amplification, an intermediate product having a dumbbell structure as shown in FIG. 2 is formed. The FIP and BIP primers bind to the F2c and B2c regions in the single-stranded loop, to initiate an elongation reaction from the 3′-terminus of the primer and from the 3′-terminus of the intermediate product. For details, reference is made to Japanese Patent No. 3313358.

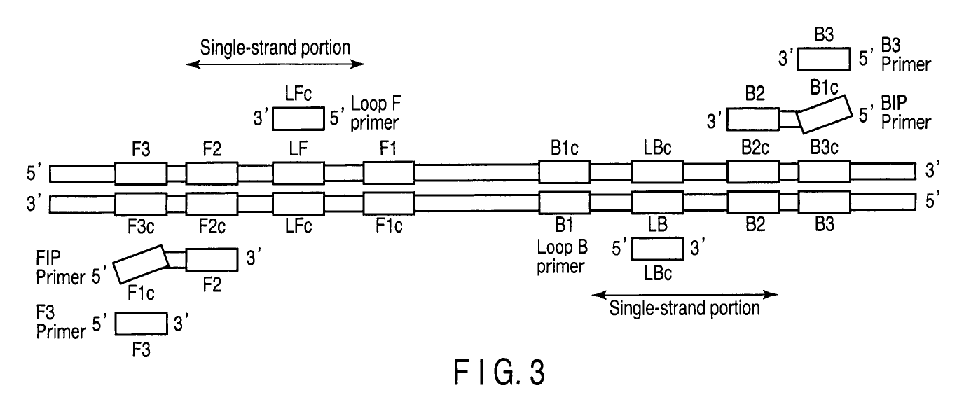

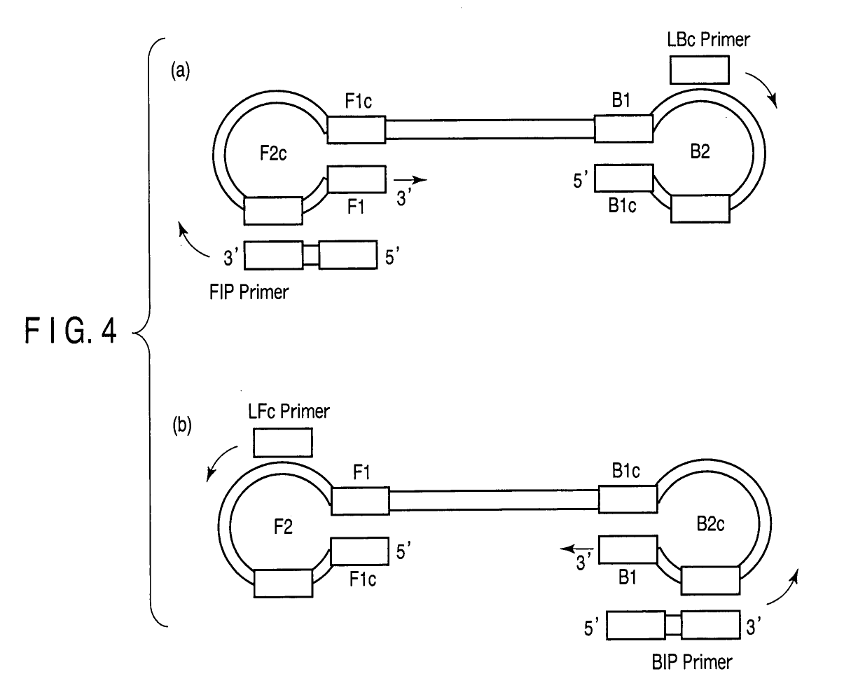

In the LAMP method, (5) a primer called a loop primer can further be arbitrarily used to reduce the amplification time. In this case, as shown in FIG. 3, an LF region is placed in a portion ranging from the F2 region to F1 region, and an LBc region is placed in a portion ranging from the B2c region to B1c region. These portions are referred to as loop primer regions. Then, a loop primer LFc consisting of a complementary sequence to the LF region, and a loop primer LBc consisting of the same sequence as the LBc region are used in addition to the 4 kinds of primers described above. For details, reference is made to WO2002/0249028. The loop primers LFc and LBc may be simultaneously used, or only one of them may be used. This loop primer, as shown in FIG. 4, anneals on a loop different from the loops annealed by the FIP and BIP primers, to provide a further synthetic initial point to promote amplification.

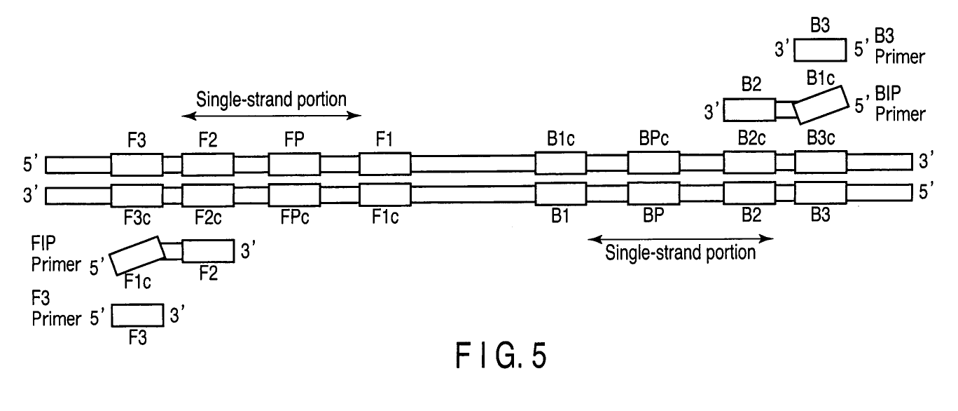

When a nucleotide polymorphism is to be detected, a polymorphic site to be detected is located in the FP region (F-loop) or BPc region (B-loop) shown in FIG. 5. Alternatively, different polymorphisms may be located in the FP and BPc regions respectively. As described in FIG. 5, the portion ranging from the F2 region to F1 region is a portion to be made single-stranded in the amplification product. Similarly, the portion ranging from the B2c region to B1c region is also a portion to be made single-stranded in the amplification product. When the polymorphic site to be detected is located in a portion to be single-stranded, its detection with nucleic acid probes can be facilitated with a high efficiency of reaction with the nucleic acid probes.

Selection of these primers will be described later in detail.

<Detection of LAMP Amplification Products; Nucleic Acid Probes>

The nucleic acid probe is designed so as to bind to the FP or BPc region containing the polymorphic site. That is, the nucleic acid probe has a sequence complementary to a sequence of a region which, in the FP or BPc region, contains the polymorphic site.

FPc and BP regions that are complementary to the FP and BPc regions, respectively, are also present in the amplification product. Accordingly, these FPc and BP regions can also be used for detection.

In this specification, a nucleic acid probe containing a sequence complementary to an amplification product of the wild type is referred to as a wild-type nucleic acid probe or a drug-nonresistant probe, while a nucleic acid probe containing a sequence complementary to an amplification product of the mutant type is referred to as a mutant-type nucleic acid probe or a drug-resistant probe.

The nucleic acid probe includes, but is not limited to, DNA, RNA, PNA, LNA, a nucleic acid having a methyl phosphonate skeleton, and other artificial nucleic acids. For immobilization on a substrate, the terminus of the nucleic acid probe may be modified with a reactive functional group such as an amino group, a carboxyl group, a hydroxyl group, a thiol group or a sulfone group. A spacer may be introduced into between the functional group and the polynucleotide. For example, a spacer consisting of an alkane or ethylene glycol skeleton may be used.

The length of the nucleic acid probe is from 15 bases at a minimum to 45 bases at a maximum. The length is more preferably 15 to 40 bases, even more preferably 18 to 35 bases.

<Nucleic Acid Probe-Immobilized Substrate>

The nucleic acid probe can be used by immobilization on a substrate, but use of the nucleic acid probe is not limited thereto. The nucleic acid probe-immobilized substrate may be a device called a DNA chip or DNA microarray known per se.

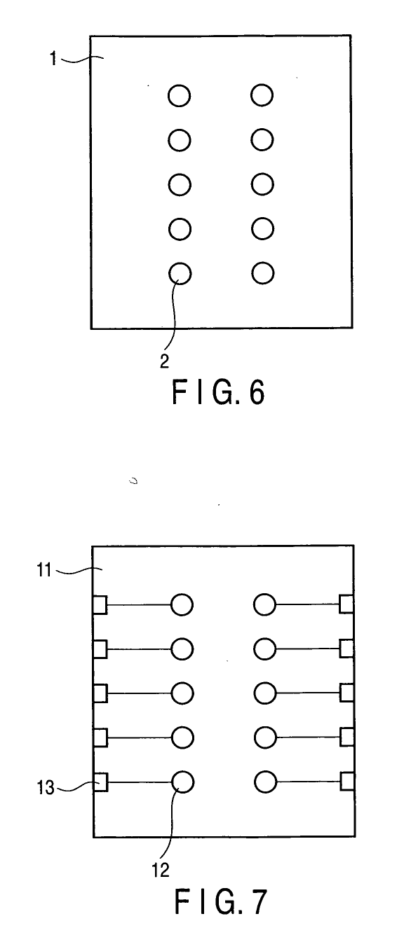

A schematic diagram of the probe-immobilized substrate in one embodiment is shown in FIG. 6. The probe is immobilized in an immobilization region 2 on a substrate 1. The substrate 1 can be produced for example from a silicon substrate or the like, but the material of the substrate is not limited thereto. The probe may be immobilized by a means known in the art. One probe or a plurality of probes may be immobilized on one substrate 1, and the arrangement and number of probes may be suitably designed and changed as necessary by those skilled in the art. When the probe is fluorescently detected as described later, the probe-immobilized substrate such as in this embodiment may be used.

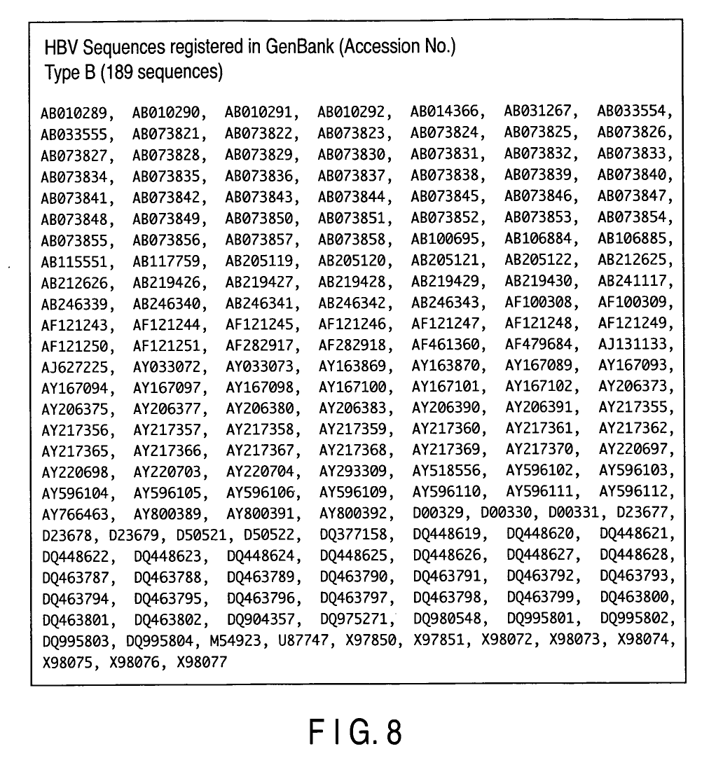

A schematic diagram of the probe-immobilized substrate in another embodiment is shown in FIG. 7. In this embodiment, a substrate 11 is provided with an electrode 12. The probe is immobilized on the electrode 12. The electrode 12 is connected to a pad 13 for retrieving electrical information. The substrate 11 can be produced for example from a silicon substrate or the like, but the material of the substrate is not limited thereto. Production of the electrode and immobilization of the probe may be conducted by a means known in the art. The electrode is not particularly limited, but may be produced from a single metal or an alloy thereof such as gold, a gold alloy, silver, platinum, mercury, nickel, palladium, silicon, germanium, gallium or tungsten, carbon such as graphite or glassy carbon, or an oxide or compound thereof.

The immobilization substrate in FIG. 7 has 10 electrodes, but the number of electrodes arranged on one substrate is not limited to this and may be arbitrarily changed. The pattern of electrodes arranged thereon is not limited to that shown in the figure and may be suitably designed and changed as necessary by those skilled in the art. The substrate 1 may be provided if necessary with a reference electrode and a counter electrode. When the probe is electrochemically detected as described later, the probe-immobilized substrate such as in this embodiment may be used.

<Hybridization Between the Nucleic Acid Probe and the Amplification Product>

Hybridization between the nucleic acid probe and the amplification product is conducted under suitable conditions. Suitable conditions vary depending on the type and structure of the amplification product, the type of bases contained in the detection sequence, and the type of the nucleic acid probe. Hybridization is conducted for example in a buffer solution with an ionic strength in the range of 0.01 to 5 and in the range of pH 5 to 10. Dextran sulfate that is a hybridization accelerator, salmon sperm DNA, calf thymus DNA, EDTA and a surfactant may be added to the reaction solution. The reaction is carried out for example at a temperature in the range of 10 to 90° C., and the efficiency of the reaction may be increased with stirring or shaking. For washing after the reaction, a buffer solution with an ionic strength in the range of 0.01 to 5 and in the range of pH 5 to 10, for example, may be used.

<Detection Method>

When the probe immobilized on the substrate is hybridized with the amplification product, a double-stranded nucleic acid is formed. This double-stranded nucleic acid can be electrochemically or fluorescently detected.

(a) Electric Current Detection System

A method of electrochemically detecting a double-stranded nucleic acid is described. In this method, a double-stranded chain-recognizing substance that specifically recognizes a double-stranded nucleic acid is used. Examples of the double-stranded chain-recognizing substance include, but are not limited to, Hoechst 33258, acridine orange, quinacrine, daunomycin, a metallointercalator, a bisintercalator such as bisacridine, a trisintercalator, and a polyintercalator. These substances may further be modified with an electrochemically active metal complex such as ferrocene or viologen.

The concentration of the double-stranded chain-recognizing substance varies depending on its type, but is generally in the range of 1 ng/mL to 1 mg/mL. In this case, a buffer solution with an ionic strength of 0.001 to 5 and in the range of pH 5 to 10 may be used.

During or after the hybridization reaction, the double-stranded chain-recognizing substance is added to the reaction solution. When a double-stranded nucleic acid has been formed by hybridization, the double-stranded chain-recognizing substance binds thereto. It follows that by applying a voltage equal to or higher than the voltage causing an electrochemical reaction of the double-stranded chain-recognizing substance, a reaction current value derived from the double-stranded chain-recognizing substance can be measured. In this case, constant-rate voltage, pulsed voltage or constant voltage may be applied. In measurement, the current and voltage may be regulated by using apparatuses such as a potentiostat, a digital multi-meter and a function generator. For example, a known electrochemical detection means described in Jpn. Pat. Appln. KOKAI Publication No. 10-146183 can be preferably used.

(b) Fluorescence Detection Method

A method of fluorescently detecting a double-stranded nucleic acid is described. A primer is previously labeled with a fluorescently active substance. Alternatively, a secondary probe labeled with a fluorescently active substance is used in detection. Alternatively, a plurality of labels may be used. The fluorescently active substance includes, but is not limited to, fluorescent dyes such as FITC, Cy3, Cy5 and rhodamine. The fluorescent substance is detected for example with a fluorescence detector. An appropriate detector adapted to the type of label is used to detect the labeled detection sequence or secondary probe.

<Guidelines for Selection of Nucleic Acid Primers and Nucleic Acid Probes>

As shown in FIG. 5, when a nucleotide polymorphic site in question is located between B1c and B2c, that is, in the BPc region, then the BIP primer is a primer having a sequence complementary to the B2c region and having the same sequence as the B1c region. Accordingly, various primers can be designed to produce objective amplification products, as far as the B2c region and B1c region are located such that the nucleotide polymorphic site in question is sandwiched therebetween.

Similarly, when a nucleotide polymorphic site in question is located between F2 and F1, that is, in the FP region, then the FIP primer is a primer having a sequence complementary to the F1 region and having the same sequence as the F2 region. Accordingly, various primers can be designed to produce objective amplification products, as far as the F1 region and F2 region are located such that the nucleotide polymorphic site in question is sandwiched therebetween.

However, it was revealed by the present inventors that the efficiency of amplification in the LAMP method varies depending on the type of primer. For example, 4 types of primers are illustrated in Tables 1, 2 and 3 shown later. These primers (FIP, BIP, F3 and B3 primers) are used in amplification. As a result, amplification with any primers is completed smoothly in a short time. Accordingly, these primers are good primers for amplification.

To further detect the amplification product with a nucleic acid probe, hybridization between the amplification product and the nucleic acid probe should be generated with high efficiency. Hence, whether the amplification product is excellent in hybridization efficiency or not is also considered to evaluate the primers.

Another pair of inner primers between which the nucleotide polymorphism is not sandwiched is designed preferably in a region of preferably 450 by or less in length, more preferably 350 by or less, anywhere between F2 and B2. Both the inner primers are designed such that the length of the single-stranded loop is preferably 100 by or less in length, more preferably 70 by or less.

Unspecific amplification with the primers is a phenomenon observed often in the LAMP method. The FIP primer includes the F1c and F2 regions, thus forming a long-chain nucleic acid. Similarly, the BIP primer includes the B1c and B2 regions, thus forming a long-chain nucleic acid. Accordingly, the FIP primers or BIP primers become entwined with each other, or the FIP primer becomes entwined with the BIP primer, thus increasing the probability of amplification with the primers as the template. In the LAMP reaction, the F3 primer, the B3 primer, and optionally the LFc primer and the LBc primer are present in the reaction solution, thus increasing the probability of unspecific reaction in LAMP reaction as compared with PCR reaction. When such unspecific reaction is generated, the amount of desired LAMP products with the sample nucleic acid as the template is decreased.

<Design of Nucleic Acid Primers and Nucleic Acid Probes>

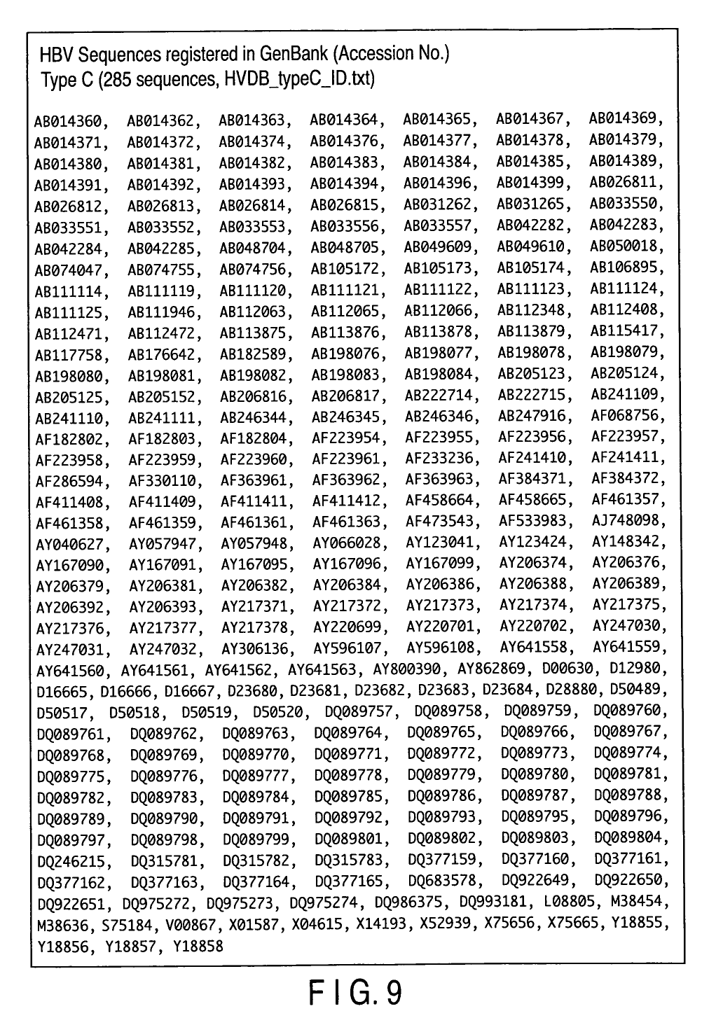

Based on the foregoing, nucleotide sequences of nucleic acid primers for LAMP amplification in accordance with the present invention, and nucleotide sequences of nucleic acid probes for detecting the LAMP amplification products, were established in the following manner. First, their standard sequences were established on the basis of a database. First, from gene sequence information database Genbank (http://www.ncbi.nlm.nih.gov./Genbank/index.html), sequence information on HBV types B and C were obtained. Accession numbers of sequences used in establishment of the standard sequences are shown in FIGS. 8 and 9.

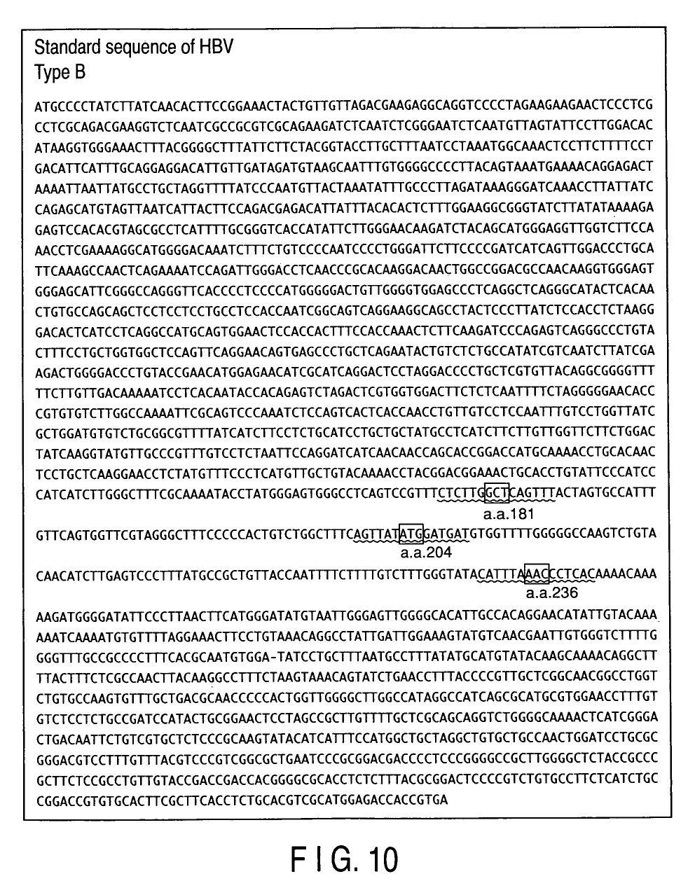

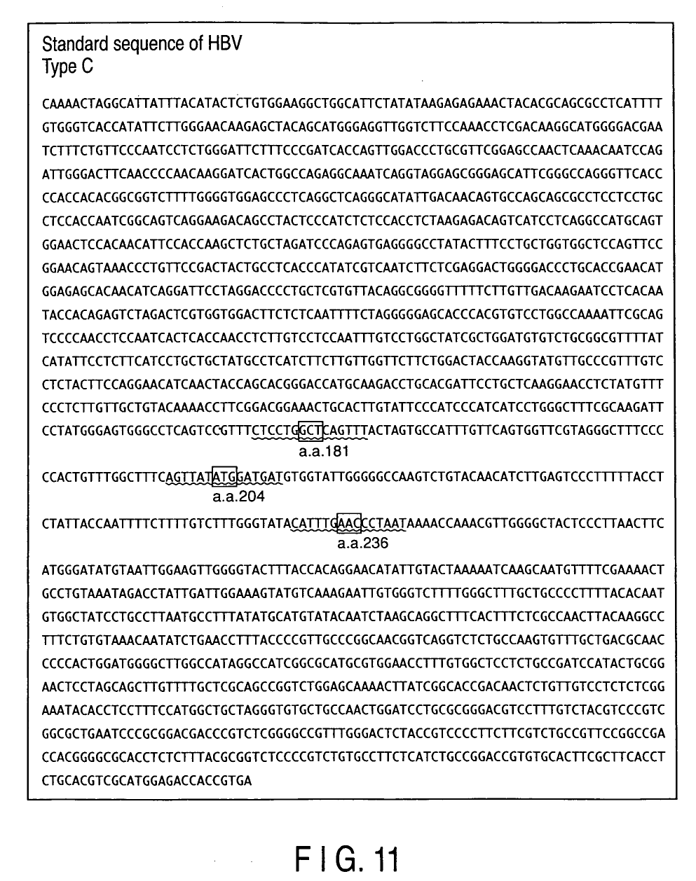

By alignment analysis of HBV types B and C, bases occurring with the highest frequency in the respective positions of the nucleotide sequences were selected as a standard sequence. The standard sequences of types B and C are shown in FIGS. 10 and 11 respectively. On the basis of this standard sequence, nucleotide sequences of 12 types of primer sets (primer sets 1 to 12) and nucleic acid probes in accordance with the present invention were determined respectively.

<Design of the Primers>

[FIP Primer (1) and BIP Primer (3)]

First, the FIP primer and BIP primer were determined. Table 1 shows FIP primers and BIP primers in 12 types of nucleic acid primer sets for detection of HBV drug-resistant strain.

TABLE 1
Nucleic acid primer sets for detection of HBV drug-resistant strain

| | | | | | | |
|---|---|---|---|---|---|---|
| | | | | | Detection | Detection |
| SET | FIP or | | SEQ. | Target | target | target |
| No. | BIP | Primer sequence (5′→3′) | ID. NO. | type | (F-Loop) | (B-Loop) |

| | | | | | | |
|---|---|---|---|---|---|---|
| 1 | FIP | CGAACCACTGAACAAATGGCCTTTCGCAAAATACCTATGGG | 3 | B | a.a.181 | a.a.204 |
| | BIP | CTTTCCCCCACTGTCTGGCTGTTGTACAGACTTGGCCCC | 4 | | | |
| 2 | FIP | CGAACCACTGAACAAATGGCCTTTCGCAAGATTCCTATGGG | 5 | C | | |
| | BIP | CTTTCCCCCACTGTTTGGCTTGTTGTACAGACTTGGCCCC | 6 | | | |
| 3 | FIP | TGTTGTACAGACTTGGCCCCCTTTCCCCCACTGTCTGGCT | 7 | B | a.a.204 | a.a.236 |
| | BIP | CCCTTTATGCCGCTGTTACCCCCCATCTTTTTGTTTTGTGAG | 8 | | | |
| 4 | FIP | TGTTGTACAGACTTGGCCCCCTTTCCCCCACTGTTTGGCT | 9 | C | | |
| | BIP | CCCTTTTTACCTCTATTACCCCCCAACGTTTGGTTTTATTAG | 10 | | | |
| 5 | FIP | TAAGGGAATATCCCCATCTTTTTGTGCTGTTACCAATTTTCTTTTGTC | 15 | B | a.a.236 | — |
| | BIP | GCACATTGCCACAGGAACATGGCCTGTTTACAGGAAGT | 16 | | | |
| 6 | FIP | TAAGGGAGTAGCCCCAACGTACCAATTTTCTTTTGTCTTTGG | 17 | C | | |
| | BIP | GGATATGTAATTGGAAGTTGGGGTATAGGTCTATTTACAGGCAGT | 18 | | | |
| 7 | FIP | ACATCATCCATATAACTGAAAGCCAGTTTACTAGTGCCATTTGTTCA | 19 | B | — | a.a.236 |
| | BIP | CCGCTGTTACCAATTTTCTTTTGTCCCAATTACATATCCCATGAAGT | 20 | | | |
| 8 | FIP | ACATCATCCATATAACTGAAAGCCATGCCATTTGTTCAGTGGT | 21 | C | | |
| | BIP | ACATCTTGAGTCCCTTTTTACCTCTGTTAAGGGAGTAGCCCCA | 22 | | | |
| 9 | FIP | TGATGGGATGGGAATACAGGTGAACCTCTATGTTTCCCTCATG | 23 | B | — | a.a.181 |
| | BIP | CTTGGGCTTTCGCAAAATACCTAAAGCCCTACGAACCACT | 24 | | | |
| 10 | FIP | TGATGGGATGGGAATACAAGTGCCACCTCTATGTTTCCCTCTT | 25 | C | | |
| | BIP | TGGGCTTTCGCAAGATTCCTCGAACCACTGAACAAATGG | 26 | | | |
| 11 | FIP | TGTTGTACAGACTTGGCCCCCTTTCCCCCACTGTCTGGCT | 7 | B | a.a.204 | a.a.236 |
| | BIP | TTACCAATTTTCTTTTGTCTTTGGGATTACATATCCCATGAAGTTAAGG | 121 | | | |
| 12 | FIP | TGTTGTACAGACTTGGCCCCCTTTCCCCCACTGTTTGGCT | 9 | C | | |
| | BIP | TTACCAATTTTCTTTTGTCTTTGGGATTACATATCCCATGAAGTTAAGG | 122 | | | |

In the table, “SET No.” indicates primer set number; “FIP or BIP” indicates that the primer is FIP primer or BIP primer; “SEQ. ID. NO.” indicates sequence number assigned to each probe; “Target Type” indicates that the detection target of the primer is either HBV type B or C; and “Detection Target” indicates the position of an amino acid to which a nucleic acid containing the target base mutation site corresponds. By using such FIP primer and BIP primer, a LAMP amplification product (also generally called a target nucleic acid or target DNA) having, in its loop, a nucleotide polymorphism site encoding the detection target amino acid can be obtained.

As shown in Table 1, each of primer sets 1 to 4, 11 and 12 is targeted to nucleotide polymorphisms at 2 positions to be detected. That is, an amplification product containing nucleotide polymorphisms at 2 positions can be obtained by amplification of a sample nucleic acid. For example, an amplification product containing a polymorphism at position 181 in the F-loop and a polymorphism at position 204 in the B-loop is obtained with the primer set 1. On the other hand, an amplification product containing a nucleotide polymorphism at 1 site is obtained with each of primer sets 5 to 10, as shown in Table 1.

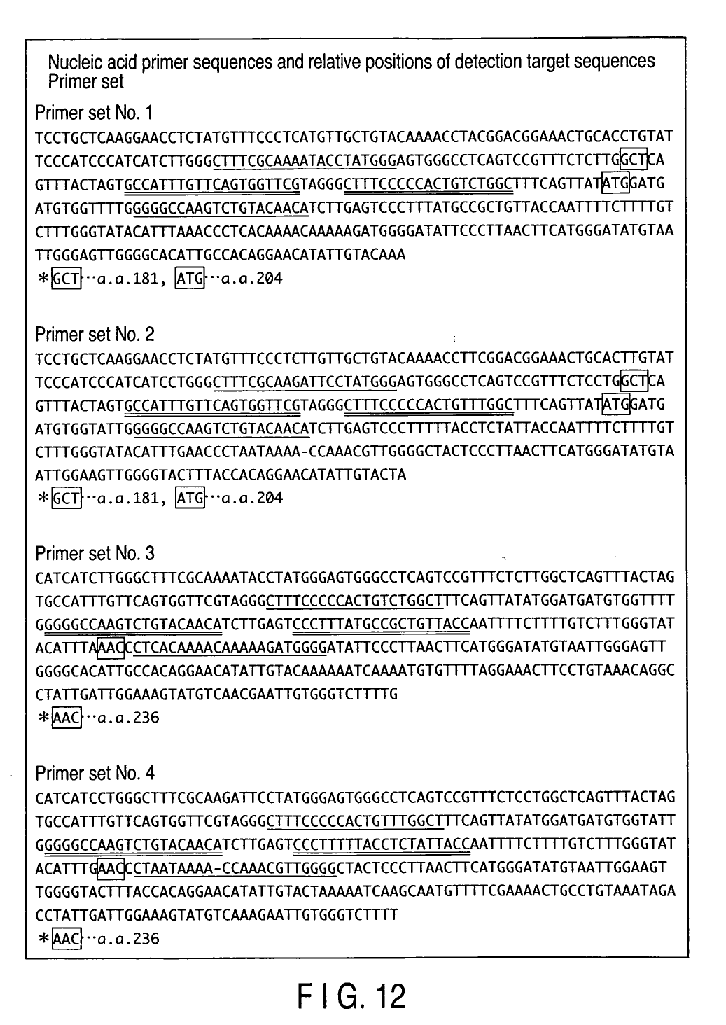

FIG. 12 shows sequence regions corresponding to primer sets 1 to 4 for example and the relative positional relationship thereof to detection target amino acids.

Bracketed 3 bases are a codon region encoding the detection target amino acid. In the primer set 1, the region “GCT” in brackets is a region encoding an amino acid at position 181. Similarly, the region “ATG” in brackets is a region encoding an amino acid at position 204. In the primer set 2, the region “GCT” in brackets is a region encoding an amino acid at position 181, and the region “ATG” in brackets is a region encoding an amino acid at position 204. In the primer set 3, the region “AAC” in brackets is a region encoding an amino acid at position 236. In the primer set 4, the region “AAC” in brackets is a region encoding an amino acid at position 236.

The single underline indicates F2 and B2 regions used in design of inner primers (FIP and BIP), and the double underline indicates F1 and B1 regions used in design of inner primers (FIP and BIP).

As long as the primers shown in Table 1 can maintain their functions as primers shown therein, bases located at positions other than the positions of the polymorphic bases may be partially substituted; further bases may be added to a site other than the positions of the polymorphic bases; or bases at positions other than the positions of the polymorphic bases may be partially deleted.

[F3 Primer (2) and B3 Primer (4)]

F3 and B3 primers may be those having sequences binding to a region upstream from the 5′-terminal of the F2 region and to a region downstream from the 3′-terminal of the B2c region. The sequences shown in Table 2 are used as F3 and B3 primer sets for each of the primer sets shown in Table 1.

TABLE 2
F3 and B3 Primer candidates

| | | | Target | |
|---|---|---|---|---|
| Primer | | | primer | SEQ. |
| No. | F or B | Primer sequence | set | ID. NO. |
| 1 | F3 | TGCACCTGTATTCCCATCCC | 1 | 27 |
| 2 | B3 | ACAGCGGCATAAAGGGACTC | | 28 |
| 3 | F3 | TGCACTTGTATTCCCATCCC | 2 | 29 |
| 4 | B3 | AATAGAGGTAAAAAGGGACTC | | 30 |
| 5 | F3 | GCCATTTGTTCAGTGGTTCG | 3, 4 | 31 |
| 6 | B3 | CATATCCCATGAAGTTAAGGG | | 32 |
| 7 | F3 | TCTTGAGTCCCTTTATGCC | 5 | 33 |
| 8 | B3 | ACCCACAATTCGTTGACA | | 34 |
| 9 | F3 | CAAGTCTGTACAACATCTTGA | 6 | 35 |
| 10 | B3 | TCTCTGACATACTTTCCAATCA | | 36 |
| 11 | F3 | CCTCAGTCCGTTTCTCTTG | 7 | 37 |
| 12 | B3 | TGTTCCTGTGGCAATGTG | | 38 |
| 13 | F3 | TCAGTCCGTTTCTCCTGG | 8 | 39 |
| 14 | B3 | ACTTCCAATTACATATCCCATG | | 40 |
| 15 | F3 | CACAACTCCTGCTCAAGG | 9 | 41 |
| 16 | B3 | CTGAAAGCCAGACAGTGG | | 42 |
| 17 | F3 | CACGATTCCTGCTCAAGG | 10 | 43 |
| 18 | B3 | ATATAACTGAAAGCCAAACAGT | | 44 |
| 24 | F3 | GCCATTTGTTCAGTGGTTCG | 11 | 31 |
| 25 | B3 | ATGTGCCCCAACTCCCA | | 123 |
| 26 | F3 | GCCATTTGTTCAGTGGTTCG | 12 | 31 |
| 27 | B3 | GTAAAGTACCCCAACTTCCA | | 124 |

In the table, “Primer No.” indicates primer number; “F or B” indicates that the probe is F3 primer or B3 primer; “Primer Sequence” indicates the nucleotide sequence of each primer; “SEQ. ID. NO.” indicates sequence number assigned to each primer; and

“Target Primer Set” indicates preferably combined primer-set number in Table 1.

As long as the primers shown in Table 2 can maintain their functions as primers shown therein, bases located at positions other than the positions of the polymorphic bases may be partially substituted; further bases may be added to sites other than the positions of the polymorphic bases; or bases at positions other than the positions of the SNP may be partially deleted.

[Loop Primer (5)]

For the purpose of improving amplification efficiency, a loop primer may be added to each primer set. According to the present invention, a sequence shown in, for example, Table 3 may be used as the loop primer.

TABLE 3
Loop primer candidates

| | | | Target | SEQ. |
|---|---|---|---|---|
| Primer | | | primer | ID. |
| No. | F or B | Primer sequence | set | NO. |
| 19 | F | CGGACTGAGGCCCACTC | 1 or 2 | 45 |
| 20 | F | AGAAACGGACTGAGGCCCAC | | 46 |
| 21 | B | CAGTTATATGGATGATGTGG | | 47 |
| 22 | B | CCAATTTTCTTTTGTCTTTGGG | 3 or 4 | 48 |
| 23 | F | CCACATCATCCATATAACTG | | 49 |
| 28 | B | CAAAAAGATGGGGATATTCC | 11 or 12 | 125 |
| 29 | B | CCAAACGTTGGGGCTAC | | 126 |

In the table, “Primer No.” indicates primer number; “F or B” indicates that the probe is F loop primer or B loop primer; “Primer Sequence” indicates the nucleotide sequence of each primer; “SEQ. ID. NO.” indicates sequence number assigned to each probe; and “Target Primer Set” indicates preferably combined primer-set number in Table 1.

As long as the primers shown in Table 3 can maintain their functions as primers shown therein, bases located at positions other than the positions of the polymorphic bases may be partially substituted; further bases may be added to sites other than the positions of the polymorphic bases; or bases at positions other than the positions of the polymorphic bases may be partially deleted.

<Design of the Probes>

Probes were designed on the basis of the standard sequences shown in FIGS. 10 and 11. Bracketed 3 bases are located in a codon region encoding an amino acid at a mutation site, that is, position 181, position 204 or position 236. As a matter of course, sequences of the bracketed 3 bases are made different based on a base mutation. Among the nucleic acid probes used in the present invention, a probe corresponding to the target amino acid at position 181, position 204 or position 236 in type B or C is selected depending on its intended detection target.

Examples of sequence regions essential for the probe sequences used in the present invention are shown in Table 4.

TABLE 4
Sequences necessary for nucleic acid probes for detection of HBV drug-resistant strain

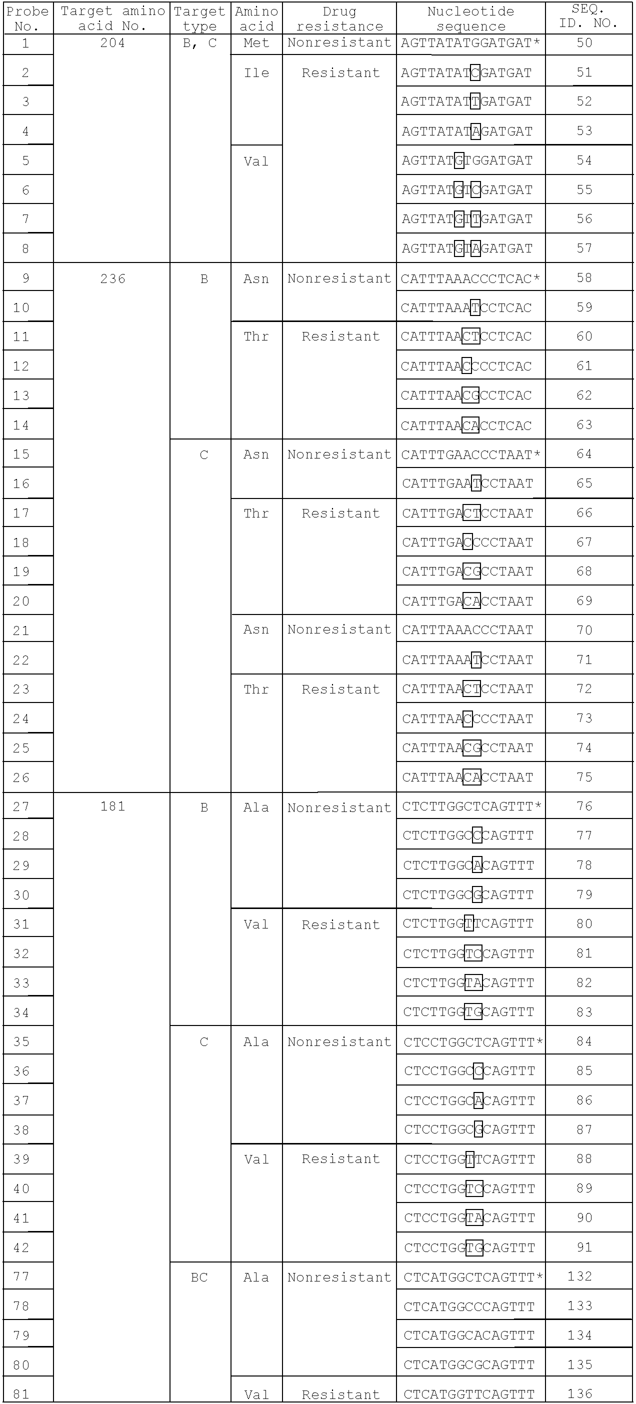

TABLE 5

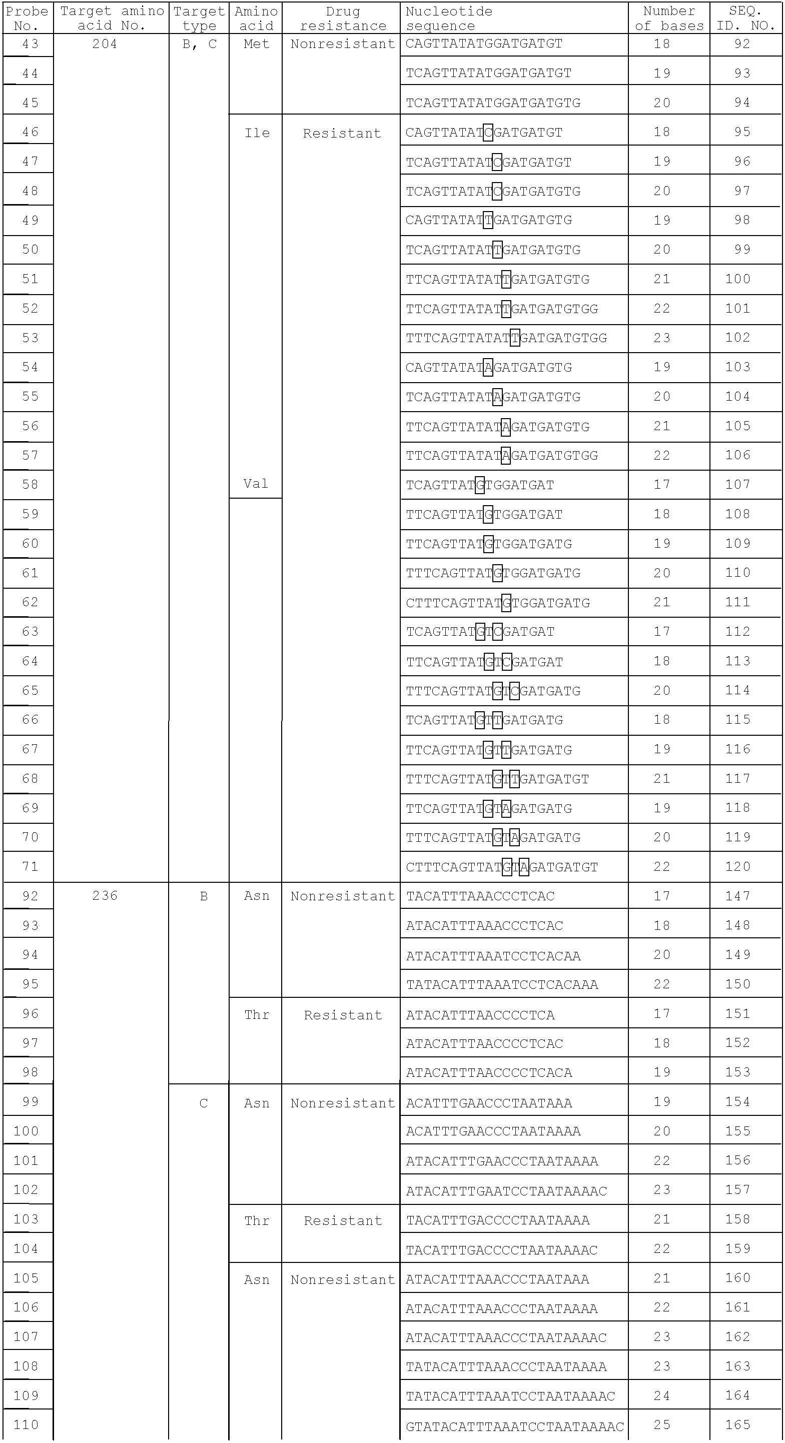

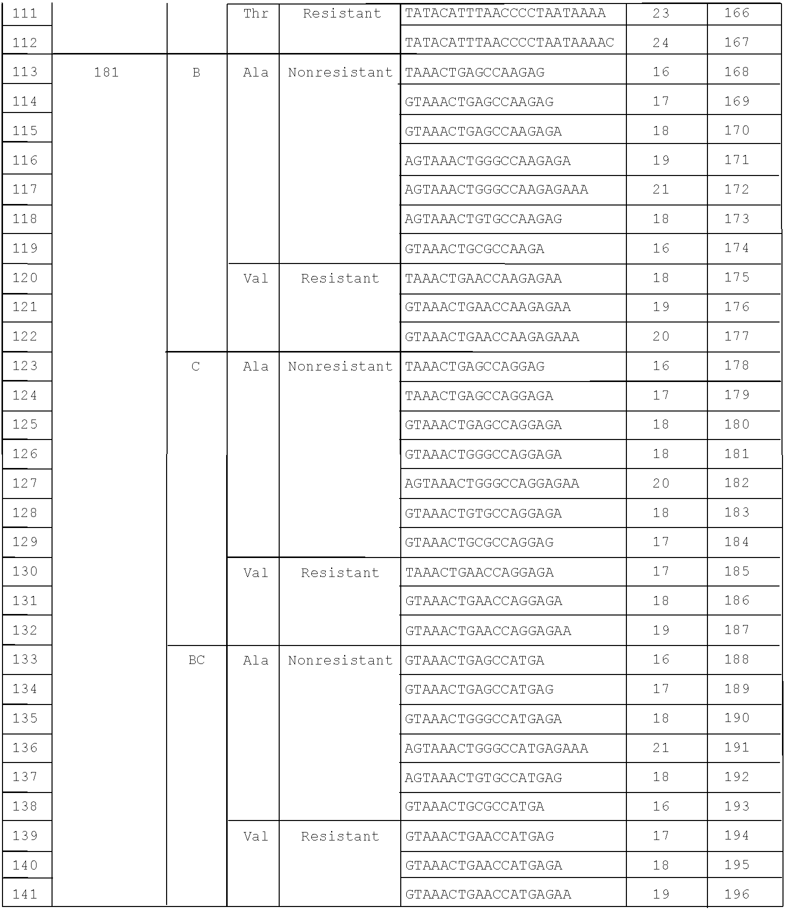

In the table, “Probe No.” indicates probe number, “Target Amino Acid No.” indicates that the mutation site to be detected is position 181, position 204 or position 236, “Target Type” indicates that HBV is B type or C type, “Amino Acid” indicates the type of the detection target amino acid, “Drug Resistance” indicates that the probe is a non-resistant probe (that is, a drug-nonresistant probe) or a resistant probe (that is, a drug-resistant probe), “Nucleotide Sequence” indicates a nucleotide sequence essential for each probe, and “SEQ. ID. NO.” indicates sequence number assigned to each probe. In Table 4, the sequence provided with * is the same nucleotide sequence as the standard sequence shown in FIG. 10 or 11.

The nucleic acid probe of the present invention is a nucleic acid chain of 15 to 45 bases in full length containing a nucleotide sequence shown in Table 4 or its complementary strand. The “nucleic acid chain of 15 to 45 bases in full length containing a nucleotide sequence shown in Table 4 or its complementary strand” is more specifically a nucleotide sequence of consecutive 15 to 45 bases, or a complementary chain thereof, that is located in the standard sequence in FIG. 10 for the probe for B-type detection or in the standard sequence in FIG. 11 for the probe for C-type detection, wherein the wavy-line portion is replaced preferably by each sequence shown in Table 4.

Among the nucleic acid chains containing sequences shown in Table 4 or complementary strands thereof, more preferable sequences are shown in Table 5.

Probes consisting of polynucleotides represented by SEQ ID NOS: 92 to 120 among the sequences shown in Table 5, or probes consisting of complementary strands thereof, are probe sequences capable of more effectively determine, under limited detection conditions, whether a nucleotide sequence encoding an amino acid at position 204 in the polymerase of viral DNA in a sample exhibits drug resistance or drug nonresistance. Particularly if it is assumed that each probe DNA is immobilized in a solid phase, then reacted with a target nucleic acid (LAMP product amplified from viral DNA with any of primer sets 1 to 4, 11 and 12), and washed in 0.2×SSC solution at 37° C., then the nucleotide sequence encoding an amino acid at position 204 in the polymerase can be clearly identified preferably by using a probe consisting of a polynucleotide represented by SEQ ID NO: 93 or its complementary strand, a probe consisting of a polynucleotide represented by SEQ ID NO: 96 or its complementary strand, a probe consisting of a polynucleotide represented by SEQ ID NO: 99 or its complementary strand, a probe consisting of a polynucleotide represented by SEQ ID NO: 105 or its complementary strand, a probe consisting of a polynucleotide represented by SEQ ID NO: 108 or its complementary strand, a probe consisting of a polynucleotide represented by SEQ ID NO: 113 or its complementary strand, a probe consisting of a polynucleotide represented by SEQ ID NO: 117 or its complementary strand, or a probe consisting of a polynucleotide represented by SEQ ID NO: 119 or its complementary strand.

Probes consisting of polynucleotides represented by SEQ ID NOS: 147 to 167 among the sequences shown in Table 5, or probes consisting of complementary strands thereof, are probe sequences capable of more effectively determine, under limited detection conditions, whether a nucleotide sequence encoding an amino acid at position 236 in the polymerase of viral DNA in a sample exhibits drug resistance or drug nonresistance. Particularly if it is assumed that each probe DNA is immobilized in a solid phase, then reacted with a target nucleic acid (LAMP product amplified from viral DNA with any of primer sets 3, 4, 5, 6, 7, 8, 11 and 12) and washed in 0.2×SSC solution at 37° C., then the nucleotide sequence encoding an amino acid at position 236 in the polymerase can be clearly identified preferably by using a probe consisting of a polynucleotide represented by SEQ ID NO: 147 or its complementary strand, a probe consisting of a polynucleotide represented by SEQ ID NO: 149 or its complementary strand, a probe consisting of a polynucleotide represented by SEQ ID NO: 151 or its complementary strand, a probe consisting of a polynucleotide represented by SEQ ID NO: 155 or its complementary strand, a probe consisting of a polynucleotide represented by SEQ ID NO: 157 or its complementary strand, a probe consisting of a polynucleotide represented by SEQ ID NO: 158 or its complementary strand, a probe consisting of a polynucleotide represented by SEQ ID NO: 161 or its complementary strand, a probe consisting of a polynucleotide represented by SEQ ID NO: 164 or its complementary strand, or a probe consisting of a polynucleotide represented by SEQ ID NO: 167 or its complementary strand.

Probes consisting of polynucleotides represented by SEQ ID NOS: 168 to 196 among the sequences shown in Table 5, or probes consisting of complementary strands thereof, are probe sequences capable of more effectively determine, under limited detection conditions, whether a nucleotide sequence encoding an amino acid at position 181 in the polymerase of viral DNA in a sample exhibits drug resistance or drug nonresistance. Particularly if it is assumed that each probe DNA is immobilized in a solid phase, then reacted with a target nucleic acid (LAMP product amplified from viral DNA with any of primer sets 1, 2, 9 and 10) and washed in 0.2×SSC solution at 37° C., then the nucleotide sequence encoding an amino acid at position 181 in the polymerase can be clearly identified preferably by using a probe consisting of a polynucleotide represented by SEQ ID NO: 169 or its complementary strand, a probe consisting of a polynucleotide represented by SEQ ID NO: 171 or its complementary strand, a probe consisting of a polynucleotide represented by SEQ ID NO: 173 or its complementary strand, a probe consisting of a polynucleotide represented by SEQ ID NO: 176 or its complementary strand, a probe consisting of a polynucleotide represented by SEQ ID NO: 179 or its complementary strand, a probe consisting of a polynucleotide represented by SEQ ID NO: 181 or its complementary strand, a probe consisting of a polynucleotide represented by SEQ ID NO: 183 or its complementary strand, a probe consisting of a polynucleotide represented by SEQ ID NO: 184 or its complementary strand, a probe consisting of a polynucleotide represented by SEQ ID NO: 186 or its complementary strand, a probe consisting of a polynucleotide represented by SEQ ID NO: 188 or its complementary strand, a probe consisting of a polynucleotide represented by SEQ ID NO: 190 or its complementary strand, a probe consisting of a polynucleotide represented by SEQ ID NO: 192 or its complementary strand, a probe consisting of a polynucleotide represented by SEQ ID NO: 193 or its complementary strand, or a probe consisting of a polynucleotide represented by SEQ ID NO: 195 or its complementary strand.

As long as the sequences shown in Table 4 or 5 can maintain their functions as the probes used in the present invention, bases located at positions other than the positions of the polymorphic bases may be partially substituted; further bases may be added to sites other than the positions of the polymorphic bases; or bases at positions other than the positions of the polymorphic bases may be partially deleted.

As the probe, a probe corresponding to the target amino acid at one of 3 sites, that is, positions 181, 204 and 236 may be selected depending on its desired detection target, and preferably a plurality of probes including different bases at the site of the polymorphic bases are simultaneously used. For example, when a nucleotide polymorphism encoding the target amino acid at position 204 is detected, probes (probe group 1) containing polynucleotides represented by SEQ ID NOS: 50 to 57 and SEQ ID NOS: 92 to 120 or complementary strands thereof are used. When a nucleotide polymorphism encoding the target amino acid at position 236 in types B and C is detected, probes (probe group 2) containing polynucleotides represented by SEQ ID NOS: 58 to 75 and SEQ ID NOS: 147 to 167 or complementary strands thereof are used. When a nucleotide polymorphism encoding the target amino acid at position 181 in types B and C is detected, probes (probe group 3) containing polynucleotides represented by SEQ ID NOS: 76 to 91, SEQ ID NOS: 132 to 136, and SEQ ID NOS: 168 to 196 or complementary strands thereof are used. When a nucleotide polymorphism encoding the target amino acids at position 181, 204 or 236 in types B and C is detected, the probe groups 1 to 3 are simultaneously used.

In the sequence information of the above database, there is a possibility that additional sequences are registered, or there is no guarantee that uniform base frequency in a specific population is shown, and therefore, it is assumed that the standard sequence changes with time or depending on the target population. Accordingly, the standard sequences mentioned above are provisional sequences so that as the sequence information is changed, the sequences shown in Tables 1 to 5 are also desirably changed in accordance therewith. In consideration of this, the sequence used may have 80% or more homology with the above sequence or have homology to such a degree as to generate an amplification reaction with HBV. Nucleotide sequences containing the sequences in Tables 1 to 4 may be used as the nucleic acid primers or nucleic acid probes even if they have fewer bases by elimination of partial bases or have more bases by addition of a peripheral sequence obtained by reference to the standard range. When there is high-frequency mutation, type-specific mutation or the like, mixed bases (that is, a mixture of plural types of bases) or modified bases may be introduced into the nucleic acid primer sequence or nucleic acid probe sequence.

<Use of Each Primer Set>

When the primer sets 1 to 12 are used to amplify a sample nucleic acid by LAMP, the amplification may be carried out by using the primer sets 1 to 12 alone or a plurality of primer sets selected from the primer sets 1 to 12, depending on the genotype or the type of polymorphic bases to be detected.

That is, the primer sets 1 to 12 may be used as a combination of a plurality of primer sets so as to detect all positions 181, 204 and 236 or may be used alone or as a combination of a plurality of primer sets so as to detect at least one of positions 181, 204 and 236. The primer sets 1 to 12 may be used as a combination thereof so as to detect both genotypes B and C or may be used alone or as a combination of a plurality of primer sets so as to detect either type B or C.

The combination of a plurality of primer sets includes combinations of primer sets of the same genotype, primer sets for the same nucleotide polymorphism in the detection target, or primers sets for the same nucleotide polymorphism in the detection target and the same position thereof (F-loop or B-loop), depending on their object, among which primers sets for the same nucleotide polymorphism in the detection target and the same position thereof (F-loop or B-loop) are desirably combined in consideration of improvements in the efficiency of amplification and the efficiency of detection. For example, a combination of primer sets 1 and 2, primer sets 3 and 4, primer sets 5 and 6, primer sets 7 and 8, primer sets 9 and 10, or primer sets 11 and 12 is a combination of primer sets for the same nucleotide polymorphism in the detection target and the same position thereof (F-loop or B-loop).

For detection of a plurality of genotypes or different nucleotide polymorphisms, LAMP amplification is conducted by using the primer sets 1 to 12 alone or in combination thereof to yield amplification products, and separate LAMP amplification is further conducted to yield amplification products. The amplification products obtained in both the amplifications may be mixed and subjected to hybridization with the nucleic acid probes.

Examples of desirable embodiments excellent in amplification efficiency and detection efficiency wherein the detection targets are positions 181, 204 and 236 in types B and C are follows:

(A) A sample nucleic acid is subjected to LAMP amplification reaction with a primer mixture of primer sets 1 and 2 (detection targets: positions 181 and 204 in types B and C) to yield amplification products (first amplification products). Separately, the sample nucleic acid is subjected to LAMP amplification reaction with a primer mixture of primer sets 3 and 4 (detection targets: positions 204 and 236 in types B and C) to yield amplification products (second amplification products). The first and second amplification products are mixed, and the mixture is subjected to hybridization with the probes for detection of the positions 181, 204 and 236 in types B and C.

(B) A sample nucleic acid is subjected to LAMP amplification reaction with a primer mixture of primer sets 1 and 2 (detection targets: positions 181 and 204 in types B and C) to yield amplification products (first amplification products). Separately, the sample nucleic acid is subjected to LAMP amplification reaction with a primer mixture of primer sets 5 and 6 (detection target: position 236 in types B and C) to yield amplification products (second amplification products). The first and second amplification products are mixed, and the mixture is subjected to hybridization with the probes for detection of the positions 181, 204 and 236 in types B and C.

(C) A sample nucleic acid is subjected to LAMP amplification reaction with a primer mixture of primer sets 1 and 2 (detection targets: positions 181 and 204 in types B and C) to yield amplification products (first amplification products). Separately, the sample nucleic acid is subjected to LAMP amplification reaction with a primer mixture of primer sets 7 and 8 (detection target: position 236 in types B and C) to yield amplification products (second amplification products). The first and second amplification products are mixed, and the mixture is subjected to hybridization with the probes for detection of the positions 181, 204 and 236 in types B and C.

(D) A sample nucleic acid is subjected to LAMP amplification reaction with a primer mixture of primer sets 1 and 2 (detection targets: positions 181 and 204 in types B and C) to yield amplification products (first amplification products). Separately, the sample nucleic acid is subjected to LAMP amplification reaction with a primer mixture of primer sets 11 and 12 (detection targets: positions 204 and 236 in types B and C) to yield amplification products (second amplification products). The first and second amplification products are mixed, and the mixture is subjected to hybridization with the probes for detection of the positions 181, 204 and 236 in types B and C.

(E) A sample nucleic acid is subjected to LAMP amplification reaction with a primer mixture of primer sets 3 and 4 (detection targets: positions 204 and 236 in types B and C) to yield amplification products (first amplification products). Separately, the sample nucleic acid is subjected to LAMP amplification reaction with a primer mixture of primer sets 9 and 10 (detection targets: position 181 in types B and C) to yield amplification products (second amplification products). The first and second amplification products are mixed, and the mixture is subjected to hybridization with the probes for detection of the positions 181, 204 and 236 in types B and C.

(F) A sample nucleic acid is subjected to LAMP amplification reaction with a primer mixture of primer sets 9 and 10 (detection target: position 181 in types B and C) to yield amplification products (first amplification products). Separately, the sample nucleic acid is subjected to LAMP amplification reaction with a primer mixture of primer sets 11 and 12 (detection targets: positions 204 and 236 in types B and C) to yield amplification products (second amplification products). The first and second amplification products are mixed, and the mixture is subjected to hybridization with the probes for detection of the positions 181, 204 and 236 in types B and C.

### Examples

Examples of the nucleic acid primer sets for LAMP amplification in accordance with the present invention and examples of the probe sets are shown in below.

### Example 1

### Use of Primer Sets 1, 2, 3 and 4

When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1C regions are placed in this order from the 3′-terminal side of the other single-stranded nucleic acid, then,

the FIP primer set contains a primer represented by SEQ ID NO: 3, a primer represented by SEQ ID NO: 5, a primer represented by SEQ ID NO: 7 and a primer represented by SEQ ID NO: 9,

the BIP primer set contains a primer represented by SEQ ID NO: 4, a primer represented by SEQ ID NO: 6, a primer represented by SEQ ID NO: 8 and a primer represented by SEQ ID NO: 10,

the F3 primer set contains a primer represented by SEQ ID NO: 27, a primer represented by SEQ ID NO: 29 and a primer represented by SEQ ID NO: 31, and

the B3 primer set contains a primer represented by SEQ ID NO: 28, a primer represented by SEQ ID NO: 30 and a primer represented by SEQ ID NO: 32.

Further, a loop primer set may be simultaneously used. In the above case, a combination of primers used preferably as loop primers is a primer represented by SEQ ID NO: 45 and/or a primer represented by SEQ ID NO: 46, and a primer represented by SEQ ID NO: 47, as well as a primer represented by SEQ ID NO: 48 and a primer represented by SEQ ID NO: 49.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 64, a probe represented by SEQ ID NO: 65, a probe represented by SEQ ID NO: 66, a probe represented by SEQ ID NO: 67, a probe represented by SEQ ID NO: 68, a probe represented by SEQ ID NO: 69, a probe represented by SEQ ID NO: 70, a probe represented by SEQ ID NO: 71, a probe represented by SEQ ID NO: 72, a probe represented by SEQ ID NO: 73, a probe represented by SEQ ID NO: 74, a probe represented by SEQ ID NO: 75, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 84, a probe represented by SEQ ID NO: 85, a probe represented by SEQ ID NO: 86, a probe represented by SEQ ID NO: 87, a probe represented by SEQ ID NO: 88, a probe represented by SEQ ID NO: 89, a probe represented by SEQ ID NO: 90, a probe represented by SEQ ID NO: 91, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the loop primer set and the probes.

### Example 2

### Use of Primer Sets 1, 2, 5 and 6

When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1c regions are placed in this order from the 3′-terminal side of the other single-stranded nucleic acid, then,

the FIP primer set contains a primer represented by SEQ ID NO: 3, a primer represented by SEQ ID NO: 5, a primer represented by SEQ ID NO: 15 and a primer represented by SEQ ID NO: 17,

the BIP primer set contains a primer represented by SEQ ID NO: 4, a primer represented by SEQ ID NO: 6, a primer represented by SEQ ID NO: 16 and a primer represented by SEQ ID NO: 18,

the F3 primer contains a primer represented by SEQ ID NO: 27, a primer represented by SEQ ID NO: 29, a primer represented by SEQ ID NO: 33 and a primer represented by SEQ ID NO: 35, and

the B3 primer contains a primer represented by SEQ ID NO: 28, a primer represented by SEQ ID NO: 30, a primer represented by SEQ ID NO: 34 and a primer represented by SEQ ID NO: 36.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 64, a probe represented by SEQ ID NO: 65, a probe represented by SEQ ID NO: 66, a probe represented by SEQ ID NO: 67, a probe represented by SEQ ID NO: 68, a probe represented by SEQ ID NO: 69, a probe represented by SEQ ID NO: 70, a probe represented by SEQ ID NO: 71, a probe represented by SEQ ID NO: 72, a probe represented by SEQ ID NO: 73, a probe represented by SEQ ID NO: 74, a probe represented by SEQ ID NO: 75, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 84, a probe represented by SEQ ID NO: 85, a probe represented by SEQ ID NO: 86, a probe represented by SEQ ID NO: 87, a probe represented by SEQ ID NO: 88, a probe represented by SEQ ID NO: 89, a probe represented by SEQ ID NO: 90, a probe represented by SEQ ID NO: 91, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the probes.

### Example 3

### Use of Primer Sets 1, 2, 7 and 8

When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1c regions are placed in this order from the 3′-terminal side of the other single-stranded nucleic acid, then,

the FIP primer set contains a primer represented by SEQ ID NO: 3, a primer represented by SEQ ID NO: 5, a primer represented by SEQ ID NO: 19 and a primer represented by SEQ ID NO: 21,

the BIP primer set contains a primer represented by SEQ ID NO: 4, a primer represented by SEQ ID NO: 6, a primer represented by SEQ ID NO: 20 and a primer represented by SEQ ID NO: 22,

the F3 primer contains a primer represented by SEQ ID NO: 27, a primer represented by SEQ ID NO: 29, a primer represented by SEQ ID NO: 37 and a primer represented by SEQ ID NO: 39, and

the B3 primer contains a primer represented by SEQ ID NO: 28, a primer represented by SEQ ID NO: 30, a primer represented by SEQ ID NO: 38 and a primer represented by SEQ ID NO: 40.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 64, a probe represented by SEQ ID NO: 65, a probe represented by SEQ ID NO: 66, a probe represented by SEQ ID NO: 67, a probe represented by SEQ ID NO: 68, a probe represented by SEQ ID NO: 69, a probe represented by SEQ ID NO: 70, a probe represented by SEQ ID NO: 71, a probe represented by SEQ ID NO: 72, a probe represented by SEQ ID NO: 73, a probe represented by SEQ ID NO: 74, a probe represented by SEQ ID NO: 75, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 84, a probe represented by SEQ ID NO: 85, a probe represented by SEQ ID NO: 86, a probe represented by SEQ ID NO: 87, a probe represented by SEQ ID NO: 88, a probe represented by SEQ ID NO: 89, a probe represented by SEQ ID NO: 90, a probe represented by SEQ ID NO: 91, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the probes.

### Example 4

### Use of Primer Sets 1, 2, 11 and 12

When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B1C, B2c and B1c regions are placed in this order from the 3′-terminal side of the other single-stranded nucleic acid, then,

the FIP primer set contains a primer represented by SEQ ID NO: 3, a primer represented by SEQ ID NO: 5, a primer represented by SEQ ID NO: 7 and a primer represented by SEQ ID NO: 9,

the BIP primer set contains a primer represented by SEQ ID NO: 4, a primer represented by SEQ ID NO: 6, a primer represented by SEQ ID NO: 121 and a primer represented by SEQ ID NO: 122,

the F3 primer contains a primer represented by SEQ ID NO: 27, a primer represented by SEQ ID NO: 29 and a primer represented by SEQ ID NO: 31, and

the B3 primer contains a primer represented by SEQ ID NO: 28, a primer represented by SEQ ID NO: 30, a primer represented by SEQ ID NO: 123 and a primer represented by SEQ ID NO: 124.

Further, a loop primer set may be simultaneously used. In the above case, a combination of primers used preferably as loop primers is a primer represented by SEQ ID NO: 45 and/or a primer represented by SEQ ID NO: 46, and a primer represented by SEQ ID NO: 47, as well as a primer represented by SEQ ID NO: 125 and a primer represented by SEQ ID NO: 126.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 64, a probe represented by SEQ ID NO: 65, a probe represented by SEQ ID NO: 66, a probe represented by SEQ ID NO: 67, a probe represented by SEQ ID NO: 68, a probe represented by SEQ ID NO: 69, a probe represented by SEQ ID NO: 70, a probe represented by SEQ ID NO: 71, a probe represented by SEQ ID NO: 72, a probe represented by SEQ ID NO: 73, a probe represented by SEQ ID NO: 74, a probe represented by SEQ ID NO: 75, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 84, a probe represented by SEQ ID NO: 85, a probe represented by SEQ ID NO: 86, a probe represented by SEQ ID NO: 87, a probe represented by SEQ ID NO: 88, a probe represented by SEQ ID NO: 89, a probe represented by SEQ ID NO: 90, a probe represented by SEQ ID NO: 91, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the probes.

### Example 5

### Use of Primer Sets 3, 4, 9 and 10

When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1c regions are placed in this order from the 3′-terminal side of the other single-stranded nucleic acid, then,

the FIP primer set contains a primer represented by SEQ ID NO: 7, a primer represented by SEQ ID NO: 9, a primer represented by SEQ ID NO: 23 and a primer represented by SEQ ID NO: 25,

the BIP primer set contains a primer represented by SEQ ID NO: 8, a primer represented by SEQ ID NO: 10, a primer represented by SEQ ID NO: 24 and a primer represented by SEQ ID NO: 26,

the F3 primer contains a primer represented by SEQ ID NO: 31, a primer represented by SEQ ID NO: 41 and a primer represented by SEQ ID NO: 43, and

the B3 primer contains a primer represented by SEQ ID NO: 32, a primer represented by SEQ ID NO: 42 and a primer represented by SEQ ID NO: 44.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 64, a probe represented by SEQ ID NO: 65, a probe represented by SEQ ID NO: 66, a probe represented by SEQ ID NO: 67, a probe represented by SEQ ID NO: 68, a probe represented by SEQ ID NO: 69, a probe represented by SEQ ID NO: 70, a probe represented by SEQ ID NO: 71, a probe represented by SEQ ID NO: 72, a probe represented by SEQ ID NO: 73, a probe represented by SEQ ID NO: 74, a probe represented by SEQ ID NO: 75, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 84, a probe represented by SEQ ID NO: 85, a probe represented by SEQ ID NO: 86, a probe represented by SEQ ID NO: 87, a probe represented by SEQ ID NO: 88, a probe represented by SEQ ID NO: 89, a probe represented by SEQ ID NO: 90, a probe represented by SEQ ID NO: 91, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the probes.

### Example 6

### Use of Primer Sets 9, 10, 11 and 12

When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1C regions are placed in this order from the 3′-terminal side of the other single-stranded nucleic acid, then,

the FIP primer set contains a primer represented by SEQ ID NO: 23, a primer represented by SEQ ID NO: 25, a primer represented by SEQ ID NO: 7 and a primer represented by SEQ ID NO: 9,

the BIP primer set contains a primer represented by SEQ ID NO: 24, a primer represented by SEQ ID NO: 26, a primer represented by SEQ ID NO: 121 and a primer represented by SEQ ID NO: 122,

the F3 primer contains a primer represented by SEQ ID NO: 41, a primer represented by SEQ ID NO: 43 and a primer represented by SEQ ID NO: 31, and

the B3 primer contains a primer represented by SEQ ID NO: 42, a primer represented by SEQ ID NO: 44, a primer represented by SEQ ID NO: 123 and a primer represented by SEQ ID NO: 124.

Further, a loop primer set may be simultaneously used. In the above case, a combination of primers used preferably as loop primers is a primer represented by SEQ ID NO: 125 and a primer represented by SEQ ID NO: 126.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 64, a probe represented by SEQ ID NO: 65, a probe represented by SEQ ID NO: 66, a probe represented by SEQ ID NO: 67, a probe represented by SEQ ID NO: 68, a probe represented by SEQ ID NO: 69, a probe represented by SEQ ID NO: 70, a probe represented by SEQ ID NO: 71, a probe represented by SEQ ID NO: 72, a probe represented by SEQ ID NO: 73, a probe represented by SEQ ID NO: 74, a probe represented by SEQ ID NO: 75, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 84, a probe represented by SEQ ID NO: 85, a probe represented by SEQ ID NO: 86, a probe represented by SEQ ID NO: 87, a probe represented by SEQ ID NO: 88, a probe represented by SEQ ID NO: 89, a probe represented by SEQ ID NO: 90, a probe represented by SEQ ID NO: 91, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the probes.

### Example 7

### Use of Primer Sets 1 and 3

These primer sets may be those detecting the drug resistance of HBV type B. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1c regions are placed in this order from the 3′-terminal side of the other single-stranded nucleic acid, then,

the FIP primer set contains a primer represented by SEQ ID NO: 3 and a primer represented by SEQ ID NO: 7,

the BIP primer set contains a primer represented by SEQ ID NO: 4 and a primer represented by SEQ ID NO: 8,

the F3 primer contains a primer represented by SEQ ID NO: 27 and a primer represented by SEQ ID NO: 31, and

the B3 primer contains a primer represented by SEQ ID NO: 28 and a primer represented by SEQ ID NO: 32.

Further, a loop primer set may be simultaneously used. In the above case, a combination of primers used preferably as loop primers is a primer represented by SEQ ID NO: 45 and/or a primer represented by SEQ ID NO: 46, and a primer represented by SEQ ID NO: 47, as well as a primer represented by SEQ ID NO: 48 and a primer represented by SEQ ID NO: 49.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the loop primer set and the probes.

### Example 8

### Use of Primer Sets 2 and 4

These primer sets may be those detecting the drug resistance of HBV type C. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1c regions are placed in this order from the 3′-terminal side of the other single-stranded nucleic acid, then,

the FIP primer set contains a primer represented by SEQ ID NO: 5 and a primer represented by SEQ ID NO: 9,

the BIP primer set contains a primer represented by SEQ ID NO: 6 and a primer represented by SEQ ID NO: 10,

the F3 primer contains a primer represented by SEQ ID NO: 29 and a primer represented by, SEQ ID NO: 31, and

the B3 primer contains a primer represented by SEQ ID NO: 30 and a primer represented by SEQ ID NO: 32.

Further, a loop primer set may be simultaneously used. In the above case, a combination of primers used preferably as loop primers is a primer represented by SEQ ID NO: 45 and/or a primer represented by SEQ ID NO: 46, and a primer represented by SEQ ID NO: 47, as well as a primer represented by SEQ ID NO: 48 and a primer represented by SEQ ID NO: 49.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 64, a probe represented by SEQ ID NO: 65, a probe represented by SEQ ID NO: 66, a probe represented by SEQ ID NO: 67, a probe represented by SEQ ID NO: 68, a probe represented by SEQ ID NO: 69, a probe represented by SEQ ID NO: 70, a probe represented by SEQ ID NO: 71, a probe represented by SEQ ID NO: 72, a probe represented by SEQ ID NO: 73, a probe represented by SEQ ID NO: 74, a probe represented by SEQ ID NO: 75, a probe represented by SEQ ID NO: 84, a probe represented by SEQ ID NO: 85, a probe represented by SEQ ID NO: 86, a probe represented by SEQ ID NO: 87, a probe represented by SEQ ID NO: 88, a probe represented by SEQ ID NO: 89, a probe represented by SEQ ID NO: 90, a probe represented by SEQ ID NO: 91, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the loop primer set and the probes.

### Example 9

### Use of Primer Sets 1 and 5

These primer sets may be those detecting the drug resistance of HBV type B. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1C regions are placed in this order from the 3′-terminal side of the other single-stranded nucleic acid, then,

the FIP primer set contains a primer represented by SEQ ID NO: 3 and a primer represented by SEQ ID NO: 15,

the BIP primer set contains a primer represented by SEQ ID NO: 4 and a primer represented by SEQ ID NO: 16,

the F3 primer set contains a primer represented by SEQ ID NO: 27 and a primer represented by SEQ ID NO: 33, and

the B3 primer set contains a primer represented by SEQ ID NO: 28 and a primer represented by SEQ ID NO: 34.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the probes.

### Example 10

### Use of Primer Sets 2 and 6

These primer sets may be those detecting the drug resistance of HBV type C. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1C regions are placed in this order from the 3′-terminal side of the other single-stranded nucleic acid, then,

the FIP primer set contains a primer represented by SEQ ID NO: 5 and a primer represented by SEQ ID NO: 17,

the BIP primer set contains a primer represented by SEQ ID NO: 6 and a primer represented by SEQ ID NO: 18,

the F3 primer set contains a primer represented by SEQ ID NO: 29 and a primer represented by SEQ ID NO: 35, and

the B3 primer set contains a primer represented by SEQ ID NO: 30 and a primer represented by SEQ ID NO: 36.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the probes.

### Example 11

### Use of Primer Sets 1 and 7

These primer sets may be those detecting the drug resistance of HBV type B. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1c regions are placed in this order from the 3′-terminal side of the other single-stranded nucleic acid, then,

the FIP primer set contains a primer represented by SEQ ID NO: 3 and a primer represented by SEQ ID NO: 19,

the BIP primer set contains a primer represented by SEQ ID NO: 4 and a primer represented by SEQ ID NO: 20,

the F3 primer set contains a primer represented by SEQ ID NO: 27 and a primer represented by SEQ ID NO: 37, and

the B3 primer set contains a primer represented by SEQ ID NO: 28 and a primer represented by SEQ ID NO: 38.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the probes.

### Example 12

### Use of Primer Sets 2 and 8

These primer sets may be those detecting the drug resistance of HBV type C. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1C regions are placed in this order from the 3′-terminal side of the other single-stranded nucleic acid, then,

the FIP primer set contains a primer represented by SEQ ID NO: 5 and a primer represented by SEQ ID NO: 21,

the BIP primer set contains a primer represented by SEQ ID NO: 6 and a primer represented by SEQ ID NO: 22,

the F3 primer set contains a primer represented by SEQ ID NO: 29 and a primer represented by SEQ ID NO: 39, and

the B3 primer set contains a primer represented by SEQ ID NO: 30 and a primer represented by SEQ ID NO: 40.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the probes.

### Example 13

### Use of Primer Sets 1 and 11

These primer sets may be those detecting the drug resistance of HBV type B. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1c regions are placed in this order from the 3′-terminal side of the other single-stranded nucleic acid, then,

the FIP primer set contains a primer represented by SEQ ID NO: 3 and a primer represented by SEQ ID NO: 7,

the BIP primer set contains a primer represented by SEQ ID NO: 4 and a primer represented by SEQ ID NO: 121,

the F3 primer contains a primer represented by SEQ ID NO: 27 and a primer represented by SEQ ID NO: 31, and

the B3 primer contains a primer represented by SEQ ID NO: 28 and a primer represented by SEQ ID NO: 123.

Further, a loop primer set may be simultaneously used. In the above case, a combination of primers used preferably as loop primers is a primer represented by SEQ ID NO: 45 and/or a primer represented by SEQ ID NO: 46, and a primer represented by SEQ ID NO: 47, as well as a primer represented by SEQ ID NO: 125.

When such primer sets are used, preferable probes used simultaneously therewith are a probe represented by SEQ ID NO: 50, a probe represented by SEQ ID NO: 51, a probe represented by SEQ ID NO: 52, a probe represented' by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the loop primer set and the probes.

### Example 14

### Use of Primer Sets 2 and 12

These primer sets may be those detecting the drug resistance of HBV type C. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B1C, B2c and B1c regions are placed in this order from the 3′-terminal side of the other single-stranded nucleic acid, then,

the FIP primer set contains a primer represented by SEQ ID NO: 5 and a primer represented by SEQ ID NO: 9,

the BIP primer set contains a primer represented by SEQ ID NO: 6 and a primer represented by SEQ ID NO: 122,

the F3 primer contains a primer represented by SEQ ID NO: 29 and a primer represented by SEQ ID NO: 31, and

the B3 primer contains a primer represented by SEQ ID NO: 30 and a primer represented by SEQ ID NO: 124.

Further, a loop primer set may be simultaneously used. In the above case, a combination of primers used preferably as loop primers is a primer represented by SEQ ID NO: 45 and/or a primer represented by SEQ ID NO: 46, and a primer represented by SEQ ID NO: 47, as well as a primer represented by SEQ ID NO: 126 and a primer represented by SEQ ID NO: 49.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 127, a probe represented by SEQ ID NO: 128, a probe represented by SEQ ID NO: 129, a probe represented by SEQ ID NO: 130, a probe represented by SEQ ID NO: 131, a probe represented by SEQ ID NO: 64, a probe represented by SEQ ID NO: 65, a probe represented by SEQ ID NO: 66, a probe represented by SEQ ID NO: 67, a probe represented by SEQ ID NO: 68, a probe represented by SEQ ID NO: 69, a probe represented by SEQ ID NO: 70, a probe represented by SEQ ID NO: 71, a probe represented by SEQ ID NO: 72, a probe represented by SEQ ID NO: 73, a probe represented by SEQ ID NO: 74, a probe represented by SEQ ID NO: 75, a probe represented by SEQ ID NO: 84, a probe represented by SEQ ID NO: 85, a probe represented by SEQ ID NO: 86, a probe represented by SEQ ID NO: 87, a probe represented by SEQ ID NO: 88, a probe represented by SEQ ID NO: 89, a probe represented by SEQ ID NO: 90, a probe represented by SEQ ID NO: 91, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the loop primer set and the probes.

### Example 15

### Use of Primer Sets 3 and 9

These primer sets may be those detecting the drug resistance of HBV type B. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1c regions are placed in this order from the 3′-terminal side of the other single-stranded nucleic acid, then,

the FIP primer set contains a primer represented by SEQ ID NO: 7 and a primer represented by SEQ ID NO: 23,

the BIP primer set contains a primer represented by SEQ ID NO: 8 and a primer represented by SEQ ID NO: 24,

the F3 primer set contains a primer represented by SEQ ID NO: 31 and a primer represented by SEQ ID NO: 41, and

the B3 primer set contains a primer represented by SEQ ID NO: 32 and a primer represented by SEQ ID NO: 42.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the probes.

### Example 16

### Use of Primer Sets 4 and 10

These primer sets may be those detecting the drug resistance of HBV type C. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1c regions are placed in this order from the 3′-terminal side of the other single-stranded nucleic acid, then,

the FIP primer set contains a primer represented by SEQ ID NO: 9 and a primer represented by SEQ ID NO: 25,

the BIP primer set contains a primer represented by SEQ ID NO: 10 and a primer represented by SEQ ID NO: 26,

the F3 primer set contains a primer represented by SEQ ID NO: 31 and a primer represented by SEQ ID NO: 33, and

the B3 primer set contains a primer represented by SEQ ID NO: 32 and a primer represented by SEQ ID NO: 44.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the probes.

### Example 17

### Use of Primer Sets 9 and 11

These primer sets may be those detecting the drug resistance of HBV type B. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1c regions are placed in this order from the 3′-terminal side of the other single-stranded nucleic acid, then,

the FIP primer set contains a primer represented by SEQ ID NO: 23 and a primer represented by SEQ ID NO: 7,

the BIP primer set contains a primer represented by SEQ ID NO: 24 and a primer represented by SEQ ID NO: 121,

the F3 primer contains a primer represented by SEQ ID NO: 41 and a primer represented by SEQ ID NO: 31, and

the B3 primer contains a primer represented by SEQ ID NO: 42 and a primer represented by SEQ ID NO: 123.

Further, a loop primer may be simultaneously used. In the above case, a sequence of a primer used preferably as loop primer is a primer represented by SEQ ID NO: 125.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 58, a probe represented by SEQ ID NO: 59, a probe represented by SEQ ID NO: 60, a probe represented by SEQ ID NO: 61, a probe represented by SEQ ID NO: 62, a probe represented by SEQ ID NO: 63, a probe represented by SEQ ID NO: 76, a probe represented by SEQ ID NO: 77, a probe represented by SEQ ID NO: 78, a probe represented by SEQ ID NO: 79, a probe represented by SEQ ID NO: 80, a probe represented by SEQ ID NO: 81, a probe represented by SEQ ID NO: 82, a probe represented by SEQ ID NO: 83, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the loop primer set and the probes.

### Example 18

### Use of Primer Sets 10 and 12

These primer sets may be those detecting the drug resistance of HBV type C. When these primer sets consist of the FIP, F3, BIP and B3 primers to detect nucleotide sequences encoding amino acids at positions 181, 204 and 236 in the polymerase region, and F3, F2 and F1 regions are placed in this order from the 5′-terminal side of one single-stranded nucleic acid of a double-stranded target nucleic acid while B3c, B2c and B1c regions are placed in this order from the 3′-terminal side of the other single-stranded nucleic acid, then,

the FIP primer set contains a primer represented by SEQ ID NO: 25 and a primer represented by SEQ ID NO: 9,

the BIP primer set contains a primer represented by SEQ ID NO: 26 and a primer represented by SEQ ID NO: 122,

the F3 primer contains a primer represented by SEQ ID NO: 43 and a primer represented by SEQ ID NO: 31, and

the B3 primer contains a primer represented by SEQ ID NO: 44 and a primer represented by SEQ ID NO: 124.

Further, a loop primer may be simultaneously used. In the above case, a sequence of a primer used preferably as loop primer is a primer represented by SEQ ID NO: 126.

When such primer sets are used, preferable probes used simultaneously therewith are a probe represented by SEQ ID NO: 50, a probe represented by SEQ ID NO: 51, a probe represented by SEQ ID NO: 52, a probe represented by SEQ ID NO: 53, a probe represented by SEQ ID NO: 54, a probe represented by SEQ ID NO: 55, a probe represented by SEQ ID NO: 56, a probe represented by SEQ ID NO: 57, a probe represented by SEQ ID NO: 64, a probe represented by SEQ ID NO: 65, a probe represented by SEQ ID NO: 66, a probe represented by SEQ ID NO: 67, a probe represented by SEQ ID NO: 68, a probe represented by SEQ ID NO: 69, a probe represented by SEQ ID NO: 70, a probe represented by SEQ ID NO: 71, a probe represented by SEQ ID NO: 72, a probe represented by SEQ ID NO: 73, a probe represented by SEQ ID NO: 74, a probe represented by SEQ ID NO: 75, a probe represented by SEQ ID NO: 84, a probe represented by SEQ ID NO: 85, a probe represented by SEQ ID NO: 86, a probe represented by SEQ ID NO: 87, a probe represented by SEQ ID NO: 88, a probe represented by SEQ ID NO: 89, a probe represented by SEQ ID NO: 90, a probe represented by SEQ ID NO: 91, a probe represented by SEQ ID NO: 132, a probe represented by SEQ ID NO: 133, a probe represented by SEQ ID NO: 134, a probe represented by SEQ ID NO: 135 and a probe represented by SEQ ID NO: 136. Alternatively, complementary strands of these probes may also be used.

Such primer sets can be used in amplification of a nucleic acid from HBV by the LAMP amplification method known per se to detect a drug-resistant strain of HBV easily and inexpensively in a short time, and their effect is augmented by using them in combination with the loop primer set and the probes.

### Example 19

The assay kit of the invention includes the primer set (optionally loop primers) in accordance with the invention and the probes in accordance with the invention. The assay kit may optionally include reagents necessary for LAMP, and may include the probes in a state immobilized on a substrate.

### Example 20

Blood, serum and an organ from a subject are used as the sample, and any of the nucleic acid primer sets for LAMP amplification and any of the probes as described above are used to examine whether a target nucleic acid from HBV is drug-resistant or drug-nonresistant.

First, a nucleic acid is extracted from the sample. The resulting sample solution is subjected to LAMP amplification with the primers shown in each of Examples 1 to 8, under conditions where suitable amplification can be attained, that is, in a suitable buffer at 60 to 65° C. under isothermal conditions.

The resulting amplification product is detected with a substrate on which the nucleic acid probes shown in each of Examples 1 to 8 were immobilized. The amplification product is subjected to hybridization by adding it to the nucleic acid-immobilized substrate, thereby electrochemically detecting the presence or absence of hybridization, to judge whether the HBV from the sample is a drug-resistant or drug-nonresistant strain.

A nucleic acid from HBV is amplified by using such primer sets in the LAMP amplification method known per se and then detected with the probes, whereby a drug-resistant strain of HBV can be detected easily and inexpensively in a short time.

### Example 21

Hereinafter, an amplification test using the primer sets 1 to 12 is described.

When a LAMP product is used as a target nucleic acid, it is first necessary to determine the arrangement of detection target bases. In the LAMP product, there are two types of loop sequences (that is, F-loop and B-loop; for example, in FIG. 2(a), the portion of F2c corresponds to F-loop and the portion of B2 to B-loop). When the detection target bases are arranged in the loop sequences, the efficiency of reaction thereof with the probes is good. To detect the detection target bases, that is, nucleotide sequences encoding amino acids at the 3 positions (positions 181, 204 and 236), in this example, the arrangement of the detection target bases in the loop sequences needs two LAMP products (totally 4 loop sequences), and the primer sets 1 to 12 are combinations for arrangement of the detection target sequences at the 3 positions in 4 loop sequences. Primer sets 1 and 2, primer sets 3 and 4, primer sets 5 and 6, primer sets 7 and 8, primer sets 9 and 10, or primer sets 11 and 12 are primer sets that are the same in the arrangement of the detection target, but are different in the target genotype (B or C). Hereinafter, an amplification test using each of the primer sets is described.

(1) Template Sequences

Two types of plasmid DNA containing the following sequences respectively were prepared as template sequences.

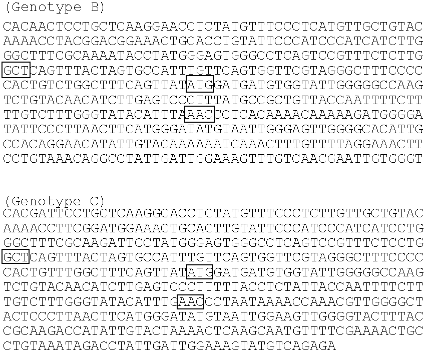

In the sequences of genotypes B and C, the 3-base codons in brackets indicate bases encoding 3 amino acids at positions 181, 204 and 236 from the upstream, respectively.

(2) Amplification of the Target Nucleic Acid by the LAMP Method

Twelve LAMP reaction solutions containing an enzyme necessary for LAMP reaction, dNTP, and a buffer solution, which contained the primer sets 1 to 12 in Table 1 respectively, were prepared. LAMP amplification was carried out at 63° C., and the rise time in LAMP amplification was detected with a turbidimeter to compare the primer sets. The “rise time” is a time in which turbidity increasing with amplification reaction is first detected with a turbidimeter.

(3) Results

The rise times in LAMP amplification obtained from the 12 primer sets are shown in Table 6.

The primer sets with which the amplification times for both genotypes B and C were less than 60 minutes were primer sets 1 and 2 or primer sets 11 and 12. Nucleotide sequences encoding aa181 and aa204 are arranged in the F-loop and B-loop respectively in each of amplification products with the primer sets 1 and 2 (see Table 1). Nucleotide sequences encoding aa204 and aa236 are arranged in the F-loop and B-loop respectively in each of amplification products with the primer sets 11 and 12 (see Table 1). The primer sets with which the amplification times for both genotypes B and C were 60 minutes to less than 70 minutes were a combination of primer sets 3 and 4 or primer sets 7 and 8. Nucleotide sequences encoding aa204 and aa236 are arranged in the F-loop and B-loop respectively in each of amplification products with the primer sets 3 and 4 (see Table 1). A nucleotide sequence encoding aa236 is arranged in the B-loop in each of amplification products with the primer sets 7 and 8 (see Table 1). The other primer sets, although showing a rise time of more than 70 minutes, are practically satisfactorily usable.

When a sequence of viral DNA is detected, the protocol is divided roughly into 3 steps: (1) DNA extraction, (2) amplification of target nucleic acid, and (3) detection of the sequence. To reduce the total time of this examination, the necessary time for each of these steps is preferably shorter, which is about 60 minutes as a standard.

Comparison among a combination of the primer sets 3 and 4, a combination of the primer sets 7 and 8 and a combination of the primer sets 11 and 12 indicates that because amplification products with the primer sets 3 and 4 or with the primer sets 11 and 12 has aa204 arranged in the F-loop, the LAMP amplification products amplified with these primer sets when combined with the primer sets 1 and 2 contain aa.204 in the loop.

When viral DNA is amplified, the amplification reaction may be inhibited by the presence of a mutation other than in the detection target. It is preferable for the nucleotide sequence of the detection target to be contained in two types or more LAMP products amplified with different primer regions in order to reduce the establishment of the above inhibition. Accordingly, it is considered more preferable that the target nucleic acid is subjected to LAMP amplification wherein aa181 (F-loop) and aa204 (B-loop) are amplified with the primer sets 1 and 2 and simultaneously aa204 (F-loop) and aa236 (B-loop) are amplified with the primer sets 3 and 4 or primer sets 11 and 12.

TABLE 6

| SET | FIP or | SEQ. | Target | Detection target | Detection target | Rise time in LAMP |
|---|---|---|---|---|---|---|
| No. | BIP | ID. NO. | type | (F-Loop) | (B-Loop) | amplification (min) |
| 1 | FIP | 3 | B | a.a.181 | a.a.204 | 42.5 |
| | BIP | 4 | | | | |
| 2 | FIP | 5 | C | | | 44.9 |
| | BIP | 6 | | | | |
| 3 | FIP | 7 | B | a.a.204 | a.a.236 | 64.0 |
| | BIP | 8 | | | | |
| 4 | FIP | 9 | C | | | 64.8 |
| | BIP | 10 | | | | |
| 5 | FIP | 15 | B | a.a.236 | — | 45.0 |
| | BIP | 16 | | | | |
| 6 | FIP | 17 | C | | | 130.0 |
| | BIP | 18 | | | | |
| 7 | FIP | 19 | B | — | a.a.236 | 61.4 |
| | BIP | 20 | | | | |
| 8 | FIP | 21 | C | | | 50.6 |
| | BIP | 22 | | | | |
| 9 | FIP | 23 | B | — | a.a.181 | 56.8 |
| | BIP | 24 | | | | |
| 10 | FIP | 25 | C | | | 71.8 |
| | BIP | 26 | | | | |
| 11 | FIP | 7 | B | a.a.204 | a.a.236 | 49.0 |
| | BIP | 121 | | | | |
| 12 | FIP | 9 | C | | | 50.7 |
| | BIP | 122 | | | | |

### Example 22

With respect to each of the preferable primer sets determined in Example 11, probe sequences for detection of each amino acid were determined.

Hereinafter, an example is described wherein the primer set 1 is used in LAMP amplification to detect a nucleotide sequence encoding an amino acid at position 204 in the polymerase region of hepatitis B virus.

(1) Template Sequences

As template sequences, the following 8 sequences were synthesized.

Template 1 has the following sequence:

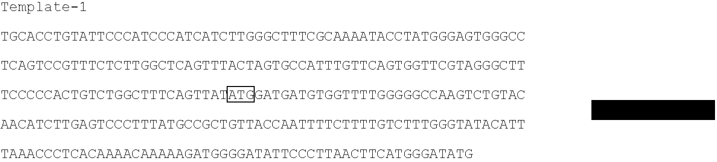

Template 2 is that nucleotide sequence of the above template 1 wherein the bracketed sequence, that is, “ATG” is replaced by “ATC”.

Template 3 is that nucleotide sequence of the above template 1 wherein the bracketed sequence, that is, “ATG” is replaced by “ATT”.

Template 4 is that nucleotide sequence of the above template 1 wherein the bracketed sequence, that is, “ATG” is replaced by “ATA”.

Template 5 is that nucleotide sequence of the above template 1 wherein the bracketed sequence, that is, “ATG” is replaced by “GTG”.

Template 6 is that nucleotide sequence of the above template 1 wherein the bracketed sequence, that is, “ATG” is replaced by “GTC”.

Template 7 is that nucleotide sequence of the above template 1 wherein the bracketed sequence, that is, “ATG” is replaced by “GTT”.

Template 8 is that nucleotide sequence of the above template 1 wherein the bracketed sequence, that is, “ATG” is replaced by “GTA”.

(2) Amplification of Target Nucleic Acid by the LAMP Method

Eight LAMP reaction solutions containing the primer set 1 (40 pmol FIP, 40 pmol BIP, 5 pmol F3, 5 pmol B3) shown in Tables 1 and 2, the loop primer 19 (20 pmol) shown in Table 3, an enzyme necessary for LAMP reaction (1 μL of Bst-DNP polymerase, manufactured by NEB), dNTP, and a buffer solution for LAMP reaction were prepared. Templates 1 to 8 were added respectively to the prepared 8 reaction solutions at a final concentration of 1E+03 copies/reaction and then reacted at 63° C. for 1 hour. Aliquots of the resulting reaction solutions were electrophoresed to confirm that LAMP products were obtained.

The remaining reaction solutions were used as LAMP product solutions to which a 20×SSC solution was then added at a final concentration of 2×SSC, to give target nucleic acid solutions. That is, the target nucleic acid solutions obtained from the templates 1 to 8 were used as the nucleic acid solutions 1 to 8 respectively. The target nucleic acids contained therein were designated target nucleic acids 1 to 8 respectively. Separately, a solution having the same composition as in the target nucleic acid solution except that it did not contain the target nucleic acid was prepared as negative control nucleic acid solution 9.

(3) Preparation of Nucleic Acid Probe-Immobilized Electrodes

In this example, a gold electrode was used as a support of a DNA probe. A substrate for DNA chip was prepared in which a working electrode having a nucleic acid probe immobilized thereon, a counter electrode and a reference electrode, both of which are necessary for electrochemical measurement, and electrode pads connected to these electrodes, are plurally arranged on one glass substrate.

Each of nucleic acid probe solutions containing nucleic acid probes consisting of the sequences of SEQ ID NOS: 92 to 120, each of which was modified with an SH group at the terminal thereof, was dropped onto the working electrode and left for 1 hour. Thereafter, the electrode was washed with ultrapure water and dried to produce a nucleic acid probe-immobilized electrode (DNA chip).

(4) Detection of the Target Nucleic Acid

Each of the target nucleic acid solutions 1 to 8 prepared above was added to the nucleic acid probe-immobilized electrode and then reacted at 45° C. for 10 minutes, thereby hybridizing the target nucleic acid with the nucleic acid probe. Thereafter, the electrode was washed by reaction for 20 minutes at each temperature (35, 37, 39° C.) with a 0.2×SSC solution, to remove the unspecifically adsorbed or bound target nucleic acid. After the washing buffer solution was removed, Hoechst 33258 was added as an intercalator capable of binding to the nucleic acid. Thereafter, a peak current value derived from the oxidation of Hoechst 33258 was calculated from a voltammogram obtained by linear sweep voltammetry.

After the target nucleic acid solution was added, the hybridization reaction at the established temperature, addition of the washing buffer solution, washing at the established temperature, addition of the intercalator, detection of the electric current and comparison of current values obtained from the respective electrodes were conducted by using a means necessary for these procedures, such as a liquid feeding regulation system, a temperature regulation system, an automatic detector including a potentiostat, and control software in each part.

(5) Results

In this example, a combination of the probe and the target nucleic acid, a part of which is completely complementary to the probe, and a combination of the probe and the target nucleic acid, a part of which is different in only 1 base from the probe, were selected, and the current value obtained from each nucleic acid probe-immobilized electrode was expressed as the ratio thereof to the background current value (S/B ratio) and shown in Tables 7 to 14.

Table 7 shows S/B ratios obtained by reacting target nucleic acid solutions 1, 2, 3, 4, 5 and 9 with probes 43 to 45; Table 8 shows S/B ratios obtained by reacting nucleic acid solutions 1, 2, 6 and 9 with probes 46 to 48; Table 9 shows S/B ratios obtained by reacting nucleic acid solutions 1, 3, 7 and 9 with probes 49 to 53; Table 10 shows S/B ratios obtained by reacting nucleic acid solutions 1, 4, 8 and 9 with probes 54 to 57; Table 11 shows S/B ratios obtained by reacting nucleic acid solutions 1, 5 and 9 with probes 58 to 62; Table 12 shows S/B ratios obtained by reacting nucleic acid solutions 2, 6 and 9 with probes 63 to 65; Table 13 shows S/B ratios obtained by reacting nucleic acid solutions 3, 7 and 9 with probes 66 to 68; and Table 14 shows S/B ratios obtained by reacting nucleic acid solutions 4, 8 and 9 with probes 69 to 71. The S/B ratio was obtained by washing at a temperature of 35, 37 or 39° C.

For example, Table 7 shows S/B ratios obtained by reacting nucleic acid solution 1, 2, 3, 4, 5 or 9 with probes 43 to 45, wherein the probes 43 to 45 are different from one another in the number of bases, have a sequence complementary to a part of the target nucleic acid 1, and have a sequence different in 1 base from complementary strands of the target nucleic acids 2, 3, 4 and 5. In each of shaded columns in each table, the used probe is completely complementary to the target nucleic acid, so these columns are parts whose values should be high. In each of other columns, on the other hand, the used probe is different in 1 base from the target nucleic acid, so these columns are parts whose values should be low.

In Table 7, it was revealed that when the washing temperature is 37° C., probe 44 shows a higher value upon reaction with the nucleic acid solution 1 and shows lower values upon reaction with the nucleic acid solutions 2, 3, 4 and 5. Accordingly, the probe 44 is preferably used when the washing temperature is 37° C.

As shown in Tables 8 to 14, numerical values were also similarly obtained with respect to the probes 46 to 71, and the most preferable number of bases was determined from the numbers of bases in the respective probe sequences.

Among probes 46 to 48, probe 47 is preferable from the result shown in Table 8; among probes 49 to 53, probe 50 is preferable from the result shown in Table 9; among probes 54 to 57, probe 56 is preferable from the result shown in Table 10; among probes 58 to 62, probe 59 is preferable from the result shown in Table 11; among probes 63 to 65, probe 64 is preferable from the result shown in Table 12; among probes 66 to 68, probe 68 is preferable from the result shown in Table 13; and among probes 69 to 71, probe 70 is preferable from the result shown in Table 14.

From these results, it was revealed that the objective target nucleic acid chains can be clearly distinguished at the washing temperature of 37° C. by a probe consisting of a nucleotide sequence represented by SEQ ID NO: 93 or its complementary strand, a probe consisting of a nucleotide sequence represented by SEQ ID NO: 96 or its complementary strand, a probe consisting of a nucleotide sequence represented by SEQ ID NO: 99 or its complementary strand, a probe consisting of a nucleotide sequence represented by SEQ ID NO: 105 or its complementary strand, a probe consisting of a nucleotide sequence represented by SEQ ID NO: 108 or its complementary strand, a probe consisting of a nucleotide sequence represented by SEQ ID NO: 113 or its complementary strand, a probe consisting of a nucleotide sequence represented by SEQ ID NO: 117 or its complementary strand, and a probe consisting of a nucleotide sequence represented by SEQ ID NO: 119 or its complementary strand.

It was also revealed that other probes shown in Table 5 can clearly distinguish the objective target nucleic acid chains when the washing temperature is changed.

TABLE 7

| | | Nucleic acid solution No. | | | | | | | | | | | | | | | | | |
|---|---|---|---|---|---|---|---|---|---|---|---|---|---|---|---|---|---|---|---|
| | | 1 | | | 2 | | | 3 | | | 4 | | | 5 | | | 9 | | |
| | | Probe No. | | | | | | | | | | | | | | | | | |
| | | 43 | 44 | 45 | 43 | 44 | 45 | 43 | 44 | 45 | 43 | 44 | 45 | 43 | 44 | 45 | 43 | 44 | 45 |
| Washing | 35 | 3.9 | 5.0 | 5.4 | 1.3 | 1.6 | 2.3 | 1.2 | 1.4 | 2.1 | 1.2 | 1.5 | 2.5 | 1.1 | 1.4 | 2.8 | | | |
| temp. | 37 | 2.6 | 4.0 | 5.2 | 1.1 | 1.4 | 1.8 | | | | 1.3 | 1.7 | 1.7 | 1.2 | 1.5 | 2.3 | 1.0 | 1.3 | 1.5 |
| | 39 | 1.8 | 2.6 | 4.5 | 1.2 | 1.5 | 1.5 | | | | 1.1 | 1.4 | 1.5 | 1.3 | 1.5 | 1.8 | | | |

TABLE 8

| | | Nucleic acid solution No. | | | | | | | | | | | |
|---|---|---|---|---|---|---|---|---|---|---|---|---|---|
| | | 1 | | | 2 | | | 6 | | | 9 | | |
| | | Probe No. | | | | | | | | | | | |
| | | 46 | 47 | 48 | 46 | 47 | 48 | 46 | 47 | 48 | 46 | 47 | 48 |
| Washing | 35 | 1.4 | 1.3 | 3.0 | 4.8 | 5.3 | 5.4 | 1.6 | 1.9 | 3.2 | | | |
| temp. | 37 | 1.4 | 1.2 | 1.3 | 3.7 | 4.3 | 4.6 | 1.3 | 1.4 | 2.2 | 1.1 | 1.1 | 1.1 |
| | 39 | 1.3 | 1.2 | 1.2 | 2.2 | 3.0 | 4.6 | 1.2 | 1.2 | 1.5 | | | |

TABLE 9

| | | Nucleic acid solution No. | | | | | | | | | | | | | | | | | | | |
|---|---|---|---|---|---|---|---|---|---|---|---|---|---|---|---|---|---|---|---|---|---|
| | | 1 | | | | | 3 | | | | | 7 | | | | | 9 | | | | |
| | | Probe No. | | | | | | | | | | | | | | | | | | | |
| | | 49 | 50 | 51 | 52 | 53 | 49 | 50 | 51 | 52 | 53 | 49 | 50 | 51 | 52 | 53 | 49 | 50 | 51 | 52 | 53 |
| Washing | 35 | 1.4 | 1.6 | 1.8 | 2.3 | 2.4 | 4.6 | 4.0 | 4.3 | 4.4 | 4.6 | 1.8 | 1.8 | 1.9 | 2.8 | 3.0 | | | | | |
| temp. | 37 | 1.2 | 1.1 | 1.3 | 1.6 | 1.6 | 4.1 | 3.5 | 3.9 | 3.9 | 4.3 | 1.3 | 1.4 | 1.6 | 1.9 | 1.9 | 1.1 | 1.1 | 1.2 | 1.1 | 1.0 |
| | 39 | 1.1 | 1.1 | 1.2 | 1.4 | 1.4 | 2.6 | 3.2 | 3.7 | 4.1 | 4.2 | 1.3 | 1.2 | 1.3 | 1.2 | 1.1 | | | | | |
| | 41 | 1.1 | 1.0 | 1.0 | 1.1 | 1.1 | 1.6 | 1.8 | 2.0 | 3.5 | 3.7 | 1.2 | 1.1 | 1.1 | 1.2 | 1.2 | | | | | |

TABLE 10

| | | Nucleic acid solution No. | | | | | | | | | | | | | | | |
|---|---|---|---|---|---|---|---|---|---|---|---|---|---|---|---|---|---|
| | | 1 | | | | 4 | | | | 8 | | | | 9 | | | |
| | | Probe No. | | | | | | | | | | | | | | | |
| | | 54 | 55 | 56 | 57 | 54 | 55 | 56 | 57 | 54 | 55 | 56 | 57 | 54 | 55 | 56 | 57 |
| Washing | 35 | 2.1 | 1.7 | 1.8 | 2.4 | 4.6 | 3.4 | 3.8 | 4.5 | 2.3 | 1.7 | 1.7 | 2.5 | | | | |
| temp. | 37 | 2.1 | 1.2 | 1.3 | 1.8 | 3.3 | 3.0 | 3.3 | 3.9 | 1.9 | 1.1 | 1.2 | 1.8 | 1.9 | 1.0 | 1.0 | 1.1 |
| | 39 | 1.9 | 1.2 | 1.1 | 1.5 | 2.3 | 1.6 | 1.8 | 3.3 | 2.0 | 1.2 | 1.1 | 1.4 | | | | |

TABLE 11

| | | Nucleic acid solution No. | | | | | | | | | | | | | | |
|---|---|---|---|---|---|---|---|---|---|---|---|---|---|---|---|---|
| | | 1 | | | | | 5 | | | | | 9 | | | | |
| | | Probe No. | | | | | | | | | | | | | | |
| | | 58 | 59 | 60 | 61 | 62 | 58 | 59 | 60 | 61 | 62 | 58 | 59 | 60 | 61 | 62 |
| Washing | 35 | 1.3 | 1.2 | 2.4 | 2.2 | 2.1 | 4.3 | 4.3 | 5.7 | 5.4 | 4.7 | | | | | |
| temp. | 37 | 1.1 | 1.1 | 1.8 | 1.7 | 1.6 | 2.8 | 3.3 | 5.3 | 4.9 | 4.5 | 1.0 | 1.0 | 1.4 | 1.4 | 1.0 |
| | 39 | 1.1 | 1.0 | 1.5 | 1.4 | 1.4 | 1.7 | 1.9 | 4.7 | 4.1 | 3.9 | | | | | |
| | 41 | 1.1 | 1.0 | 1.3 | 1.4 | 1.2 | 1.2 | 1.3 | 3.0 | 2.4 | 2.6 | | | | | |

TABLE 12

| | | Nucleic acid | | | | | | | | |
|---|---|---|---|---|---|---|---|---|---|---|
| | | solution No. | | | | | | | | |
| | | 2 | | | 6 | | | 9 | | |
| | | Probe No. | | | | | | | | |
| | | 63 | 64 | 65 | 63 | 64 | 65 | 63 | 64 | 65 |
| Washing | 35 | 1.3 | 1.4 | 2.3 | 4.6 | 4.8 | 4.8 | | | |
| temp. | 37 | 1.2 | 1.2 | 1.7 | 3.6 | 4.4 | 4.9 | 0.9 | 1.0 | 1.0 |
| | 39 | 1.2 | 1.3 | 1.4 | 2.3 | 3.0 | 4.3 | | | |

TABLE 13

| | | Nucleic acid | | | | | | | | |
|---|---|---|---|---|---|---|---|---|---|---|
| | | solution No. | | | | | | | | |
| | | 3 | | | 7 | | | 9 | | |
| | | Probe No. | | | | | | | | |
| | | 66 | 67 | 68 | 66 | 67 | 68 | 66 | 67 | 68 |
| Washing | 35 | 1.6 | 1.8 | 1.9 | 5.0 | 5.0 | 4.9 | | | |
| temp. | 37 | 1.3 | 1.4 | 1.5 | 4.1 | 4.3 | 4.2 | 0.9 | 1.0 | 1.0 |
| | 39 | 1.2 | 1.2 | 1.2 | 2.7 | 3.2 | 3.2 | | | |

TABLE 14

| | | Nucleic acid | | | | | | | | |
|---|---|---|---|---|---|---|---|---|---|---|
| | | solution No. | | | | | | | | |
| | | 4 | | | 8 | | | 9 | | |
| | | Probe No. | | | | | | | | |
| | | 69 | 70 | 71 | 69 | 70 | 71 | 69 | 70 | 71 |
| Washing | 35 | 1.6 | 1.9 | 1.9 | 4.3 | 4.5 | 4.5 | | | |
| temp. | 37 | 1.3 | 1.4 | 1.6 | 3.3 | 3.2 | 3.6 | 0.9 | 1.0 | 1.0 |
| | 39 | 1.1 | 1.3 | 1.2 | 2.0 | 2.2 | 2.6 | | | |

### Example 23

Hereinafter, an example is described wherein the primer sets 11 and 12 are used in LAMP amplification to detect a nucleotide sequence encoding an amino acid at position 236 in the polymerase region of hepatitis B and C viruses.

(1) Template Sequences

As template sequences, the following 9 sequences were synthesized.

Template 15 has the following sequence:

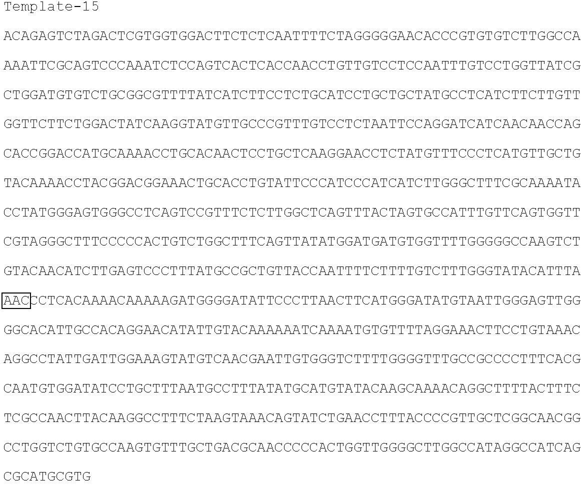

Template 16 is that nucleotide sequence of the above template 15 wherein the bracketed sequence, that is, “AAC” is replaced by “AAT”.

Template 17 is that nucleotide sequence of the above template 15 wherein the bracketed sequence, that is, “AAC” is replaced by “ACC”.

Template 18 has the following sequence:

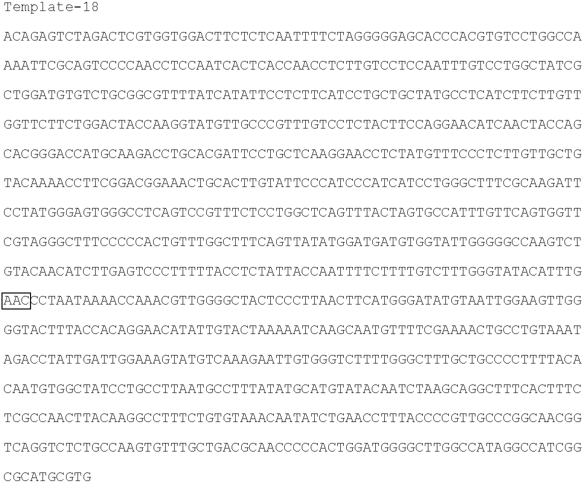

Template 19 is that nucleotide sequence of the above template 18 wherein the bracketed sequence, that is, “GAAC” is replaced by “GAAT”.

Template 20 is that nucleotide sequence of the above template 18 wherein the bracketed sequence, that is, “GAAC” is replaced by “GACC”.

Template 21 is that nucleotide sequence of the above template 18 wherein the bracketed sequence, that is, “GAAC” is replaced by “AAAC”.

Template 22 is that nucleotide sequence of the above template 18 wherein the bracketed sequence, that is, “GAAC” is replaced by “AAAI”.

Template 23 is that nucleotide sequence of the above template 18 wherein the bracketed sequence, that is, “GAAC” is replaced by “AACC”.

(2) Amplification of Target Nucleic Acid by the LAMP Method

Nine LAMP reaction solutions containing the primer sets 11 and 12 (40 pmol FIP, 40 pmol BIP, 5 pmol F3, 5 pmol B3) shown in Tables 1 and 2, the loop primers 125 and 126 (each 20 pmol) shown in Table 3, an enzyme necessary for LAMP reaction (2 μL of Bst-DNP polymerase, manufactured by NEB), dNTP, and a buffer solution for LAMP reaction were prepared. Templates 15 to 23 were added respectively to the prepared 9 reaction solutions at a final concentration of 1E+03 copies/reaction and then reacted at 63° C. for 1 hour. Aliquots of the resulting reaction solutions were electrophoresed to confirm that LAMP products were obtained.

The remaining reaction solutions were used as LAMP product solutions to which a 20×SSC solution was then added at a final concentration of 2×SSC, to give target nucleic acid solutions. That is, the target nucleic acid solutions obtained from the templates 15 to 23 were used as the nucleic acid solutions 15 to 23 respectively. The target nucleic acids contained therein were designated target nucleic acids 15 to 23, respectively. Separately, a solution having the same composition as in the target nucleic acid solution except that it did not contain the target nucleic acid was prepared as negative control nucleic acid solution 9.

(3) Preparation of Nucleic Acid Probe-Immobilized Electrodes

In this example, a gold electrode was used as a support of a DNA probe. A substrate for DNA chip was prepared in which a working electrode having a nucleic acid probe immobilized thereon, a counter electrode and a reference electrode, both of which are necessary for electrochemical measurement, and electrode pads connected to these electrodes, are plurally arranged on one glass substrate.

Each of nucleic acid probe solutions containing nucleic acid probes consisting of the sequences of SEQ ID NOS: 147 to 167, each of which was modified with an SH group at the terminal thereof, was dropped onto the working electrode and left for 1 hour. Thereafter, the electrode was washed with ultrapure water and dried to produce a nucleic acid probe-immobilized electrode (DNA chip).

(4) Detection of the Target Nucleic Acid

Each of the target nucleic acid solutions 15 to 23 prepared above was added to the nucleic acid probe-immobilized electrode and then reacted at 45° C. for 10 minutes, thereby hybridizing the target nucleic acid with the nucleic acid probe. Thereafter, the electrode was washed by reaction for 10 minutes at each temperature (35, 37, 39° C.) with a 0.2×SSC solution, to remove the unspecifically adsorbed or bound target nucleic acid. After the washing buffer solution was removed, Hoechst 33258 was added as an intercalator capable of binding to the nucleic acid. Thereafter, a peak current value derived from the oxidation of Hoechst 33258 was calculated from a voltammogram obtained by linear sweep voltammetry.

After the target nucleic acid solution was added, the hybridization reaction at the established temperature, addition of the washing buffer solution, washing at the established temperature, addition of the intercalator, detection of the electric current and comparison of current values obtained from the respective electrodes were conducted by using a means necessary for these procedures, such as a liquid feeding regulation system, a temperature regulation system, an automatic detector including a potentiostat, and control software in each part.

(5) Results

In this example, a combination of the probe and the target nucleic acid, a part of which is completely complementary to the probe, and a combination of the probe and the target nucleic acid, a part of which is different in only 1 or 2 bases from the probe, were selected, and the current value obtained from each nucleic acid probe-immobilized electrode was expressed as the ratio thereof to the background current value (S/B ratio) and shown in Tables A to 28.

Table A shows S/B ratios obtained by reacting target nucleic acid solutions 15, 17 and 9 with probes 92 and 93; Table B shows S/B ratios obtained by reacting nucleic acid solutions 16, 17 and 9 with probes 94 and 95; Table C shows S/B ratios obtained by reacting nucleic acid solutions 15, 17 and 9 with probes 96 to 98; Table D shows S/B ratios obtained by reacting nucleic acid solutions 18, 20 and 9 with probes 99 to 101; Table E shows S/B ratios obtained by reacting nucleic acid solutions 19, 20 and 9 with probe 102; Table F shows S/B ratios obtained by reacting nucleic acid solutions 18, 20 and 9 with probes 103 and 104; Table G shows S/B ratios obtained by reacting nucleic acid solutions 21, 23 and 9 with probes 105 to 108; Table H shows S/B ratios obtained by reacting nucleic acid solutions 22, 23 and 9 with probes 109 and 110; and Table I shows S/B ratios obtained by reacting nucleic acid solutions 21, 23 and 9 with probes 111 and 112. The S/B ratio was obtained by washing at a temperature of 35, 37 or 39° C.

In each of shaded columns in each table, the used probe is completely complementary to the target nucleic acid, so these columns are parts whose values should be high. In each of other columns, on the other hand, the used probe is different in 1 or 2 bases from the target nucleic acid, so these columns are parts whose values should be low.

In Table A, it was revealed that when the washing temperature is 37° C., probe 92 shows a higher value upon reaction with the nucleic acid solution 15 and shows a lower value upon reaction with the nucleic acid solution 17. Accordingly, the probe 92 is preferably used when the washing temperature is 37° C.

As shown in Tables B to I, numerical values were also similarly obtained with respect to the probes 94 to 112, and the most preferable number of bases was determined from the numbers of bases in the respective probe sequences.

Among probes 94 and 95, probe 94 is preferable from the result shown in Table B; among probes 96 to 98, probe 97 is preferable from the result shown in Table C; among probes 99 to 101, probe 99 is preferable from the result shown in Table D; probe 102 is preferable from the result shown in Table E; among probes 103 and 104, probe 103 is preferable from the result shown in Table F; among probes 105 to 108, probe 106 is preferable from the result shown in Table G; among probes 109 and 110, probe 109 is preferable from the result shown in Table H; and among probes 111 and 112, probe 112 is preferable from the result shown in Table I.

From these results, it was revealed that the objective target nucleic acid chains can be clearly distinguished at the washing temperature of 37° C. by a probe consisting of a nucleotide sequence represented by SEQ ID NO: 147 or its complementary strand, a probe consisting of a nucleotide sequence represented by SEQ ID NO: 149 or its complementary strand, a probe consisting of a nucleotide sequence represented by SEQ ID NO: 152 or its complementary strand, a probe consisting of a nucleotide sequence represented by SEQ ID NO: 155 or its complementary strand, a probe consisting of a nucleotide sequence represented by SEQ ID NO: 157 or its complementary strand, a probe consisting of a nucleotide sequence represented by SEQ ID NO: 158 or its complementary strand, a probe consisting of a nucleotide sequence represented by SEQ ID NO: 161 or its complementary strand, a probe consisting of a nucleotide sequence represented by SEQ ID NO: 164 or its complementary strand, and a probe consisting of a nucleotide sequence represented by SEQ ID NO: 167 or its complementary strand.

It was also revealed that other probes shown in Table 5 can clearly distinguish the objective target nucleic acid chains when the washing temperature is changed.

TABLE A

| Nucleic acid | | |
|---|---|---|
| solution No. | | |
| 15 | 17 | 9 |

Probe No.

| | | 92 | 93 | 92 | 93 | 92 | 93 |
|---|---|---|---|---|---|---|---|
| Washing | 35 | 4.1 | 4.3 | 1.7 | 2.6 | | |
| temp. | 37 | 3.0 | 3.8 | 1.2 | 1.7 | 1.1 | 1.0 |
| | 39 | 1.9 | 3.0 | 1.0 | 1.1 | | |

TABLE B

| Nucleic acid | | |
|---|---|---|
| solution No. | | |
| 16 | 17 | 9 |

Probe No.

| | | 94 | 95 | 94 | 95 | 94 | 95 |
|---|---|---|---|---|---|---|---|
| Washing | 35 | | | 1.5 | 2.0 | | |
| temp. | 37 | 4.5 | | 1.4 | 1.3 | 1.2 | 1.1 |
| | 39 | | | 1.4 | 1.2 | | |

TABLE C

| | | Nucleic acid | | | | | | | | |
|---|---|---|---|---|---|---|---|---|---|---|
| | | solution No. | | | | | | | | |
| | | 15 | | | 17 | | | 9 | | |
| | | Probe No. | | | | | | | | |
| | | 96 | 97 | 98 | 96 | 97 | 98 | 96 | 97 | 98 |
| Washing | 35 | 1.0 | 1.1 | 0.9 | 3.1 | 4.7 | 3.0 | | | |
| temp. | 37 | 1.1 | 1.1 | 0.8 | 2.4 | 4.4 | 3.0 | 0.9 | 1.1 | 0.8 |
| | 39 | 0.9 | 1.1 | 0.8 | 1.4 | 3.7 | 2.6 | | | |

TABLE D

| | | Nucleic acid | | | | | | | | |
|---|---|---|---|---|---|---|---|---|---|---|
| | | solution No. | | | | | | | | |
| | | 18 | | | 20 | | | 9 | | |
| | | Probe No. | | | | | | | | |
| | | 99 | 100 | 101 | 99 | 100 | 101 | 99 | 100 | 101 |
| Washing | 35 | 3.7 | 4.8 | 5.4 | 1.9 | 2.6 | 5.1 | | | |
| temp. | 37 | 2.5 | 3.7 | 5.1 | 1.1 | 1.4 | 2.9 | 1.1 | 1.4 | 1.5 |
| | 39 | 1.4 | 2.1 | 4.5 | 1.0 | 1.4 | 1.8 | | | |

TABLE E

| | | Nucleic acid solution No. | | |
|---|---|---|---|---|
| | | 19 | 20 | 9 |
| Probe No. | | 102 | 102 | 102 |
| Washing | 35 | | 3.0 | |
| temp. | 37 | 3.5 | 1.4 | 1.3 |
| | 39 | | 1.4 | |

TABLE F

| | | Nucleic acid | | | | | |
|---|---|---|---|---|---|---|---|
| | | solution No. | | | | | |
| | | 18 | | 20 | | 9 | |
| | | Probe No. | | | | | |
| | | 103 | 104 | 103 | 104 | 103 | 104 |
| Washing | 35 | 1.4 | 2.5 | 5.7 | 5.2 | | |
| temp. | 37 | 1.3 | 1.5 | 5.0 | 4.9 | 1.3 | 1.0 |
| | 39 | 1.3 | 1.1 | 4.3 | 4.7 | | |

TABLE G

| | | Nucleic acid solution No. | | | | | | | | | | | |
|---|---|---|---|---|---|---|---|---|---|---|---|---|---|
| | | 21 | | | | 23 | | | | 9 | | | |
| | | Probe No. | | | | | | | | | | | |
| | | 105 | 106 | 107 | 108 | 105 | 106 | 107 | 108 | 105 | 106 | 107 | 108 |
| Washing | 35 | 4.7 | 5.1 | 4.9 | 5.2 | 1.5 | 1.9 | 4.2 | 3.3 | | | | |
| temp. | 37 | 3.1 | 3.8 | 4.5 | 4.5 | 1.2 | 1.5 | 4.0 | 2.2 | 1.2 | 1.2 | 1.2 | 1.2 |
| | 39 | 1.8 | 2.4 | 4.3 | 3.6 | 1.2 | 1.2 | 2.4 | 1.2 | | | | |

TABLE H

| | | Nucleic acid | | | | | |
|---|---|---|---|---|---|---|---|
| | | solution No. | | | | | |
| | | 22 | | 23 | | 9 | |
| | | Probe No. | | | | | |
| | | 109 | 110 | 109 | 110 | 109 | 110 |
| Washing | 35 | | | 1.7 | 3.1 | | |
| temp. | 37 | 5.7 | | 1.3 | 2.0 | 1.3 | 1.3 |
| | 39 | | | 1.3 | 1.3 | | |

TABLE I

| | | Nucleic acid | | | | | |
|---|---|---|---|---|---|---|---|
| | | solution No. | | | | | |
| | | 21 | | 23 | | 9 | |
| | | Probe No. | | | | | |
| | | 111 | 112 | 111 | 112 | 111 | 112 |
| Washing | 35 | 1.2 | 2.8 | 4.6 | 4.9 | | |
| temp. | 37 | 1.2 | 1.3 | 4.5 | 5.0 | 1.2 | 1.2 |
| | 39 | 1.2 | 1.2 | 2.8 | 4.7 | | |

### Example 24

Hereinafter, an example is described wherein the primer sets 1 and 2 are used in LAMP amplification to detect a nucleotide sequence encoding an amino acid at position 181 in the polymerase region of hepatitis B and C viruses.

(1) Template Sequences

As template sequences, the following 15 sequences were synthesized.

Template 24 has the following sequence:

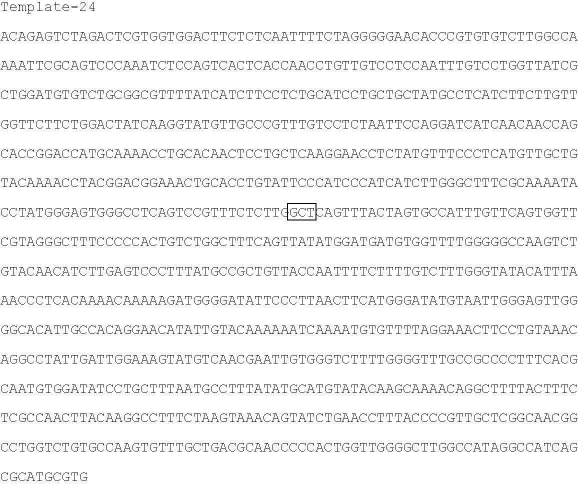

Template 25 is that nucleotide sequence of the above template 24 wherein the bracketed sequence, that is, “GCT” is replaced by “GCC”.

Template 26 is that nucleotide sequence of the above template 24 wherein the bracketed sequence, that is, “GCT” is replaced by “GCA”.

Template 27 is that nucleotide sequence of the above template 24 wherein the bracketed sequence, that is, “GCT” is replaced by “GCG”.

Template 28 is that nucleotide sequence of the above template 24 wherein the bracketed sequence, that is, “GCT” is replaced by “GTT”.

Template 29 has the following sequence:

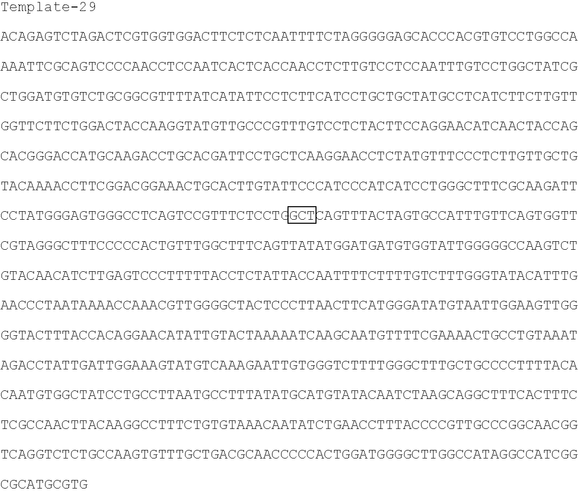

Template 30 is that nucleotide sequence of the above template 29 wherein the bracketed sequence, that is, “GCT” is replaced by “GCC”.

Template 31 is that nucleotide sequence of the above template 29 wherein the bracketed sequence, that is, “GCT” is replaced by “GCA”.

Template 32 is that nucleotide sequence of the above template 29 wherein the bracketed sequence, that is, “GCT” is replaced by “GCG”.

Template 33 is that nucleotide sequence of the above template 29 wherein the bracketed sequence, that is, “GCT” is replaced by “GTT”.

Template 34 has the following sequence:

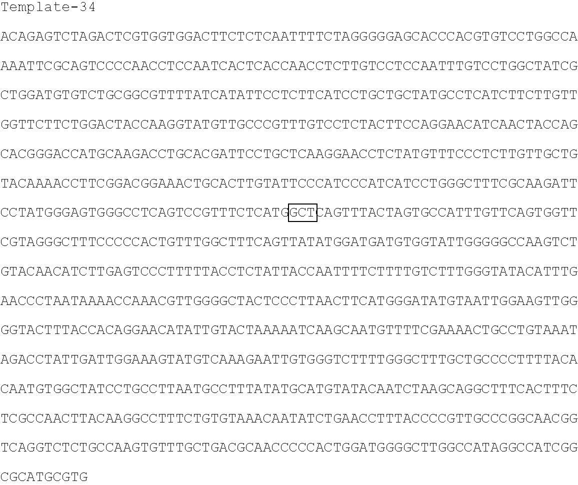

Template 35 is that nucleotide sequence of the above template 34 wherein the bracketed sequence, that is, “GCT” is replaced by “GCC”.

Template 36 is that nucleotide sequence of the above template 34 wherein the bracketed sequence, that is, “GCT” is replaced by “GCA”.

Template 37 is that nucleotide sequence of the above template 34 wherein the bracketed sequence, that is, “GCT” is replaced by “GCG”.

Template 38 is that nucleotide sequence of the above template 34 wherein the bracketed sequence, that is, “GCT” is replaced by “GTT”.

(2) Amplification of Target Nucleic Acid by the LAMP Method

Nine LAMP reaction solutions containing the primer sets 1 and 2 (40 pmol FIP, 40 pmol BIP, 5 pmol F3, 5 pmol B3) shown in Tables 1 and 2, the loop primer 46 (each 20 pmol) shown in Table 3, an enzyme necessary for LAMP reaction (2 μL of Bst-DNP polymerase, manufactured by NEB), dNTP, and a buffer solution for LAMP reaction were prepared. Templates 24 to 38 were added respectively to the prepared 9 reaction solutions at a final concentration of 1E+03 copies/reaction and then reacted at 63° C. for 1 hour. Aliquots of the resulting reaction solutions were electrophoresed to confirm that LAMP products were obtained.

The remaining reaction solutions were used as LAMP product solutions to which a 20×SSC solution was then added at a final concentration of 2×SSC, to give target nucleic acid solutions. That is, the target nucleic acid solutions obtained from the templates 24 to 38 were used as the nucleic acid solutions 24 to 38 respectively. The target nucleic acids contained therein were designated target nucleic acids 24 to 38 respectively. Separately, a solution having the same composition as in the target nucleic acid solution except that it did not contain the target nucleic acid was prepared as negative control nucleic acid solution 9.

(3) Preparation of Nucleic Acid Probe-Immobilized Electrodes

In this example, a gold electrode was used as a support of a DNA probe. A substrate for DNA chip was prepared in which a working electrode having a nucleic acid probe immobilized thereon, a counter electrode and a reference electrode, both of which are necessary for electrochemical measurement, and electrode pads connected to these electrodes, are plurally arranged on one glass substrate.

Each of nucleic acid probe solutions containing nucleic acid probes consisting of the sequences of SEQ ID NOS: 168 to 196, each of which was modified with an SH group at the terminal thereof, was dropped onto the working electrode and left for 1 hour. Thereafter, the electrode was washed with ultrapure water and dried to produce a nucleic acid probe-immobilized electrode (DNA chip).

(4) Detection of the Target Nucleic Acid

Each of the target nucleic acid solutions 24 to 38 prepared above was added to the nucleic acid probe-immobilized electrode and then reacted at 45° C. for 10 minutes, thereby hybridizing the target nucleic acid with the nucleic acid probe. Thereafter, the electrode was washed by reaction for 10 minutes at each temperature (35, 37, 39° C.) with a 0.2×SSC solution, to remove the unspecifically adsorbed or bound target nucleic acid. After the washing buffer solution was removed, Hoechst 33258 was added as an intercalator capable of binding to the nucleic acid. Thereafter, a peak current value derived from the oxidation of Hoechst 33258 was calculated from a voltammogram obtained by linear sweep voltammetry.

After the target nucleic acid solution was added, the hybridization reaction at the established temperature, addition of the washing buffer solution, washing at the established temperature, addition of the intercalator, detection of the electric current and comparison of current values obtained from the respective electrodes were conducted by using a means necessary for these procedures, such as a liquid feeding regulation system, a temperature regulation system, an automatic detector including a potentiostat, and control software in each part.

(5) Results

In this example, a combination of the probe and the target nucleic acid, a part of which is completely complementary to the probe, and a combination of the probe and the target nucleic acid, a part of which is different in only 1 or 2 bases from the probe, were selected, and the current value obtained from each nucleic acid probe-immobilized electrode was expressed as the ratio thereof to the background current value (S/B ratio) and shown in Tables J to 43.

Table J shows S/B ratios obtained by reacting target nucleic acid solutions 24, 28 and 9 with probes 113 to 115; Table K shows S/B ratios obtained by reacting nucleic acid solutions 25, 28 and 9 with probes 116 and 117; Table L shows S/B ratios obtained by reacting nucleic acid solutions 26, 28 and 9 with probe 118; Table M shows S/B ratios obtained by reacting nucleic acid solutions 27, 28 and 9 with probe 119; Table N shows S/B ratios obtained by reacting nucleic acid solutions 24, 28 and 9 with probes 120 to 122; Table O shows S/B ratios obtained by reacting nucleic acid solutions 29, 33 and 9 with probes 123 to 125; Table P shows S/B ratios obtained by reacting nucleic acid solutions 30, 33 and 9 with probes 126 and 127; Table Q shows S/B ratios obtained by reacting nucleic acid solutions 31, 33 and 9 with probe 128; Table R shows S/B ratios obtained by reacting nucleic acid solutions 32, 33 and 9 with probe 129; Table S shows S/B ratios obtained by reacting target nucleic acid solutions 29, 33 and 9 with probes 130 to 132; Table T shows S/B ratios obtained by reacting nucleic acid solutions 34, 38 and 9 with probes 133 and 134; Table U shows S/B ratios obtained by reacting nucleic acid solutions 35, 38 and 9 with probes 135 and 136; Table V shows S/B ratios obtained by reacting nucleic acid solutions 36, 38 and 9 with probe 137; Table W shows S/B ratios obtained by reacting nucleic acid solutions 37, 38 and 9 with probe 138; and Table X shows S/B ratios obtained by reacting nucleic acid solutions 34, 38 and 9 with probes 139 to 141. The S/B ratio was obtained by washing at a temperature of 35, 37 or 39° C.

In each of shaded columns in each table, the used probe is completely complementary to the target nucleic acid, so these columns are parts whose values should be high. In each of other columns, on the other hand, the used probe is different in 1 or 2 bases from the target nucleic acid, so these columns are parts whose values should be low.

In Table J, it was revealed that when the washing temperature is 37° C., probe 114 shows a higher value upon reaction with the nucleic acid solution 24 and shows a lower value upon reaction with the nucleic acid solution 28. Accordingly, the probe 114 is preferably used when the washing temperature is 37° C.

As shown in Tables K to X, numerical values were also similarly obtained with respect to the probes 116 to 141, and the most preferable number of bases was determined from the numbers of bases in the respective probe sequences.

Among probes 116 and 117, probe 116 is preferable from the result shown in Table K; probe 118 is preferable from the result shown in Table L; probe 119 is preferable from the result shown in Table M; among probes 120 to 122, probe 121 is preferable from the result shown in Table N; among probes 123 to 125, probe 124 is preferable from the result shown in Table 0; among probes 126 and 127, probe 126 is preferable from the result shown in Table P; probe 128 is preferable from the result shown in Table Q; probe 129 is preferable from the result shown in Table R; among probes 130 to 132, probe 131 is preferable from the result shown in Table S; among probes 133 and 134, probe 133 is preferable from the result shown in Table T; among probes 135 and 136, probe 135 is preferable from the result shown in Table U; probe 137 is preferable from the result shown in Table V; probe 138 is preferable from the result shown in Table W; and among probes 139 to 141, probe 140 is preferable from the result shown in Table X.

From these results, it was revealed that the objective target nucleic acid chains can be clearly distinguished at the washing temperature of 37° C. by a probe consisting of a nucleotide sequence represented by SEQ ID NO: 169 or its complementary strand, a probe consisting of a nucleotide sequence represented by SEQ ID NO: 171 or its complementary strand, a probe consisting of a nucleotide sequence represented by SEQ ID NO: 173 or its complementary strand, a probe consisting of a nucleotide sequence represented by SEQ ID NO: 174 or its complementary strand, a probe consisting of a nucleotide sequence represented by SEQ ID NO: 176 or its complementary strand, a probe consisting of a nucleotide sequence represented by SEQ ID NO: 179 or its complementary strand, a probe consisting of a nucleotide sequence represented by SEQ ID NO: 181 or its complementary strand, a probe consisting of a nucleotide sequence represented by SEQ ID NO: 183 or its complementary strand, a probe consisting of a nucleotide sequence represented by SEQ ID NO: 184 or its complementary strand, a probe consisting of a nucleotide sequence represented by SEQ ID NO: 186 or its complementary strand, a probe consisting of a nucleotide sequence represented by SEQ ID NO: 188 or its complementary strand, a probe consisting of a nucleotide sequence represented by SEQ ID NO: 190 or its complementary strand, a probe consisting of a nucleotide sequence represented by SEQ ID NO: 192 or its complementary strand, a probe consisting of a nucleotide sequence represented by SEQ ID NO: 193 or its complementary strand, and a probe consisting of a nucleotide sequence represented by SEQ ID NO: 195 or its complementary strand.

It was also revealed that other probes shown in Table 5 can clearly distinguish the objective target nucleic acid chains when the washing temperature is changed.

TABLE J

| | | Nucleic acid | | | | | | | | |
|---|---|---|---|---|---|---|---|---|---|---|
| | | solution No. | | | | | | | | |
| | | 24 | | | 28 | | | 9 | | |
| | | Probe No. | | | | | | | | |
| | | 113 | 114 | 115 | 113 | 114 | 115 | 113 | 114 | 115 |
| Washing | 35 | 2.7 | 4.1 | 3.1 | 1.1 | 2.3 | 2.3 | | | |
| temp. | 37 | 2.3 | 3.9 | 3.2 | 1.0 | 1.6 | 1.5 | 1.0 | 1.2 | 1.0 |
| | 39 | 1.6 | 3.8 | 3.3 | 0.9 | 1.3 | 1.1 | | | |

TABLE K

| | | Nucleic acid | | | | | |
|---|---|---|---|---|---|---|---|
| | | solution No. | | | | | |
| | | 25 | | 28 | | 9 | |
| | | Probe No. | | | | | |
| | | 116 | 117 | 116 | 117 | 116 | 117 |
| Washing | 35 | | | 2.2 | 3.9 | | |
| temp. | 37 | 3.3 | | 1.6 | 2.5 | 1.2 | 2.3 |
| | 39 | | | 1.2 | 2.0 | | |

TABLE L

| | | Nucleic acid solution No. | | |
|---|---|---|---|---|
| | | 26 | 28 | 9 |
| Probe No. | | 118 | 118 | 118 |
| Washing | 35 | | 1.1 | |
| temp. | 37 | 4.7 | 1.1 | 1.3 |
| | 39 | | 1.1 | |

TABLE M

| | | Nucleic acid solution No. | | |
|---|---|---|---|---|
| | | 27 | 28 | 9 |
| Probe No. | | 119 | 119 | 119 |
| Washing | 35 | | 1.0 | |
| temp. | 37 | 4.0 | 1.0 | 1.2 |
| | 39 | | 1.0 | |

TABLE N

| | | Nucleic acid solution No. | | | | | | | | |
|---|---|---|---|---|---|---|---|---|---|---|
| | | 24 | | | 28 | | | 9 | | |
| | | Probe No. | | | | | | | | |
| | | 120 | 121 | 122 | 120 | 121 | 122 | 120 | 121 | 122 |
| Washing | 35 | 0.9 | 2.0 | 2.3 | 2.5 | 4.0 | 4.0 | | | |
| temp. | 37 | 0.9 | 1.4 | 1.7 | 1.7 | 3.9 | 4.0 | 0.9 | 1.1 | 1.1 |
| | 39 | 0.9 | 1.1 | 1.1 | 1.2 | 3.2 | 3.5 | | | |

TABLE O

| | | Nucleic acid solution No. | | | | | | | | |
|---|---|---|---|---|---|---|---|---|---|---|
| | | 29 | | | 33 | | | 9 | | |
| | | Probe No. | | | | | | | | |
| | | 123 | 124 | 125 | 123 | 124 | 125 | 123 | 124 | 125 |
| Washing | 35 | 3.6 | 4.4 | 4.6 | 1.3 | 1.7 | 3.4 | | | |
| temp. | 37 | 3.4 | 4.1 | 4.5 | 1.1 | 1.4 | 2.5 | 1.1 | 1.3 | 1.2 |
| | 39 | 2.7 | 3.5 | 4.3 | 0.9 | 1.2 | 1.6 | | | |

TABLE P

| | | Nucleic acid solution No. | | | | | |
|---|---|---|---|---|---|---|---|
| | | 30 | | 33 | | 9 | |
| | | Probe No. | | | | | |
| | | 126 | 127 | 126 | 127 | 126 | 127 |
| Washing | 35 | | | 1.8 | 2.6 | | |
| temp. | 37 | 3.1 | 3.3 | 1.5 | 2.1 | 1.4 | 1.5 |
| | 39 | | | 1.2 | 1.5 | | |

TABLE Q

| | | Nucleic acid solution No. | | |
|---|---|---|---|---|
| | | 31 | 33 | 9 |
| Probe No. | | 128 | 128 | 128 |
| Washing | 35 | | 1.5 | |
| temp. | 37 | 4.4 | 1.1 | 1.1 |
| | 39 | | 1.1 | |

TABLE R

| | | Nucleic acid solution No. | | |
|---|---|---|---|---|
| | | 32 | 33 | 9 |
| Probe No. | | 129 | 129 | 129 |
| Washing | 35 | | 1.0 | |
| temp. | 37 | 3.5 | 1.0 | 1.0 |
| | 39 | | 0.9 | |

TABLE S

| | | Nucleic acid solution No. | | | | | | | | |
|---|---|---|---|---|---|---|---|---|---|---|
| | | 29 | | | 33 | | | 9 | | |
| | | Probe No. | | | | | | | | |
| | | 130 | 131 | 132 | 130 | 131 | 132 | 130 | 131 | 132 |
| Washing | 35 | 1.1 | 2.6 | 3.0 | 2.8 | 3.7 | 4.1 | | | |
| temp. | 37 | 1.0 | 1.8 | 2.3 | 2.1 | 3.4 | 3.8 | 0.9 | 1.0 | 1.2 |
| | 39 | 1.0 | 1.3 | 1.5 | 1.4 | 3.0 | 3.5 | | | |

TABLE T

| | | Nucleic acid solution No. | | | | | |
|---|---|---|---|---|---|---|---|
| | | 34 | | 38 | | 9 | |
| | | Probe No. | | | | | |
| | | 133 | 134 | 133 | 134 | 133 | 134 |
| Washing | 35 | 4.2 | 4.2 | 1.4 | 2.1 | | |
| temp. | 37 | 3.8 | 4.2 | 1.2 | 1.6 | 1.1 | 1.0 |
| | 39 | 3.0 | 4.0 | 1.1 | 1.2 | | |

TABLE U

| | | Nucleic acid solution No. | | | | | |
|---|---|---|---|---|---|---|---|
| | | 35 | | 38 | | 9 | |
| | | Probe No. | | | | | |
| | | 135 | 136 | 135 | 136 | 135 | 136 |
| Washing | 35 | | | 1.8 | 3.0 | | |
| temp. | 37 | 3.8 | | 1.5 | 2.3 | 1.3 | 1.3 |
| | 39 | | | 1.3 | 1.6 | | |

TABLE V

| | | Nucleic acid solution No. | | |
|---|---|---|---|---|
| | | 36 | 38 | 9 |
| Probe No. | | 137 | 137 | 137 |
| Washing | 35 | | 1.5 | |
| temp. | 37 | 5.0 | 1.7 | 1.5 |
| | 39 | | 1.4 | |

TABLE W

| | | Nucleic acid solution No. | | |
|---|---|---|---|---|
| | | 37 | 38 | 9 |
| Probe No. | | 138 | 138 | 138 |
| Washing | 35 | | 1.0 | |
| temp. | 37 | 4.3 | 1.0 | 1.0 |
| | 39 | | 1.0 | |

TABLE X

| | | Nucleic acid solution No. | | | | | | | | |
|---|---|---|---|---|---|---|---|---|---|---|
| | | 35 | | | 38 | | | 9 | | |
| | | Probe No. | | | | | | | | |
| | | 139 | 140 | 141 | 139 | 140 | 141 | 139 | 140 | 141 |
| Washing | 35 | 1.7 | 2.1 | 2.4 | 3.9 | 4.1 | 4.1 | | | |
| temp. | 37 | 1.2 | 1.6 | 1.7 | 3.6 | 4.1 | 4.1 | 1.0 | 1.2 | 1.2 |
| | 39 | 1.0 | 1.2 | 1.2 | 2.9 | 3.5 | 3.8 | | | |

What is claimed is:

1. A nucleic acid primer set for LAMP amplification for detection of drug-resistant and drug-nonresistant strains of hepatitis B virus, comprising an FIP primer, an F3 primer, a BIP primer and a B3 primer,
wherein the set is selected from the group consisting of:
a primer set 1 wherein the FIP primer consists of a polynucleotide represented by SEQ ID NO: 3, the BIP primer consists of a polynucleotide represented by SEQ ID NO: 4, the F3 primer consists of a polynucleotide represented by SEQ ID NO: 27, and the B3 primer consists of a polynucleotide represented by SEQ ID NO: 28, and
a primer set 2 wherein the FIP primer consists of a polynucleotide represented by SEQ ID NO: 5, the BIP primer consists of a polynucleotide represented by SEQ ID NO: 6, the F3 primer consists of a polynucleotide represented by SEQ ID NO: 29, and the B3 primer consists of a polynucleotide represented by SEQ ID NO: 30,
which are used for amplification of a nucleotide sequence region containing nucleotide sequences coding for amino acids at positions 181 and 204 in the polymerase region of hepatitis B virus;
a primer set 3 wherein the FIP primer consists of a polynucleotide represented by SEQ ID NO: 7, the BIP primer consists of a polynucleotide represented by SEQ ID NO: 8, the F3 primer consists of a polynucleotide represented by SEQ ID NO: 31, and the B3 primer consists of a polynucleotide represented by SEQ ID NO: 32,
a primer set 4 wherein the FIP primer consists of a polynucleotide represented by SEQ ID NO: 9, the BIP primer consists of a polynucleotide represented by SEQ ID NO: 10, the F3 primer consists of a polynucleotide represented by SEQ ID NO: 31, and the B3 primer consists of a polynucleotide represented by SEQ ID NO: 32,
a primer set 11 wherein the FIP primer consists of a polynucleotide represented by SEQ ID NO: 7, the BIP primer consists of a polynucleotide represented by SEQ ID NO: 121, the F3 primer consists of a polynucleotide represented by SEQ ID NO: 31, and the B3 primer consists of a polynucleotide represented by SEQ ID NO: 123, and
a primer set 12 wherein the FIP primer consists of a polynucleotide represented by SEQ ID NO: 9, the BIP primer consists of a polynucleotide represented by SEQ ID NO: 122, the F3 primer consists of a polynucleotide represented by SEQ ID NO: 31, and the B3 primer consists of a polynucleotide represented by SEQ ID NO: 124,
which are used for amplification of a nucleotide sequence region containing nucleotide sequences coding for amino acids at positions 204 and 236 in the polymerase region of hepatitis B virus;
a primer set 5 wherein the FIP primer consists of a polynucleotide represented by SEQ ID NO: 15, the BIP primer consists of a polynucleotide represented by SEQ ID NO: 16, the F3 primer consists of a polynucleotide represented by SEQ ID NO: 33, and the B3 primer consists of a polynucleotide represented by SEQ ID NO: 34,
a primer set 6 wherein the FIP primer consists of a polynucleotide represented by SEQ ID NO: 17, the BIP primer consists of a polynucleotide represented by SEQ ID NO: 18, the F3 primer consists of a polynucleotide represented by SEQ ID NO: 35, and the B3 primer consists of a polynucleotide represented by SEQ ID NO: 36,
a primer set 7 wherein the FIP primer consists of a polynucleotide represented by SEQ ID NO: 19, the BIP primer consists of a polynucleotide represented by SEQ ID NO: 20, the F3 primer consists of a polynucleotide represented by SEQ ID NO: 37, and the B3 primer consists of a polynucleotide represented by SEQ ID NO: 38, and
a primer set 8 wherein the FIP primer consists of a polynucleotide represented by SEQ ID NO: 21, the BIP primer consists of a polynucleotide represented by SEQ ID NO: 22, the F3 primer consists of a polynucleotide represented by SEQ ID NO: 39, and the B3 primer consists of a polynucleotide represented by SEQ ID NO: 40,
which are used for amplification of a nucleotide sequence region containing a nucleotide sequence coding for an amino acid at position 236 in the polymerase region of hepatitis B virus; and
a primer set 9 wherein the FIP primer consists of a polynucleotide represented by SEQ ID NO: 23, the BIP primer consists of a polynucleotide represented by SEQ ID NO: 24, the F3 primer consists⁻of a polynucleotide represented by SEQ ID NO: 41, and the B3 primer consists of a polynucleotide represented by SEQ ID NO: 42, and
a primer set 10 wherein the FIP primer consists of a polynucleotide represented by SEQ ID NO: 25, the BIP primer consists of a polynucleotide represented by SEQ ID NO: 26, the F3 primer consists of a polynucleotide represented by SEQ ID NO: 43, and the B3 primer consists of a polynucleotide represented by SEQ ID NO: 44,
which are used for amplification of a nucleotide sequence region containing a nucleotide sequence coding for an amino acid at position 181 in the polymerase region of hepatitis B virus.

2. The primer set according to claim 1, which further comprises, as a loop primer, a primer consisting of a polynucleotide represented by at least one sequence from among SEQ ID NO: 45, SEQ ID NO: 46 and SEQ ID NO: 47, for the primer set 1 or 2; a primer consisting of a polynucleotide represented by at least one sequence from among SEQ ID NO: 48 and SEQ ID NO: 49, for the primer set 3 or 4; and a primer represented by at least one sequence from among SEQ ID NO: 125 and SEQ ID NO: 126, for the primer set 11 or 12.

3. A method of detecting a drug-resistant or drug-nonresistant strain of hepatitis B virus, comprising:
amplifying a hepatitis B virus nucleic acid in a sample solution by LAMP with a primer set to yield an amplification product, and
hybridizing the amplification product with a probe containing a polynucleotide derived from a drug-resistant strain of hepatitis B virus and/or a probe containing a polynucleotide derived from a drug-nonresistant strain of hepatitis B virus, to detect a drug-resistant or drug-nonresistant strain of hepatitis B virus,
wherein the primer set comprises an FIP primer, an F3 primer, a BIP primer and a B3 primer, and at least one set is selected from the group consisting of:
a primer set 1 wherein the FIP primer consists of a polynucleotide represented by SEQ ID NO: 3, the BIP primer consists of a polynucleotide represented by SEQ ID NO: 4, the F3 primer consists of a polynucleotide represented by SEQ ID NO: 27, and the B3 primer consists of a polynucleotide represented by SEQ ID NO: 28, and
a primer set 2 wherein the FIP primer consists of a polynucleotide represented by SEQ ID NO: 5, the BIP primer consists of a polynucleotide represented by SEQ ID NO: 6, the F3 primer consists of a polynucleotide represented by SEQ ID NO: 29, and the B3 primer consists of a polynucleotide represented by SEQ ID NO: 30,
which are used for amplification of a nucleotide sequence region containing nucleotide sequences coding for amino acids at positions 181 and 204 in the polymerase region of hepatitis B virus;
a primer set 3 wherein the FIP primer consists of a polynucleotide represented by SEQ ID NO: 7, the BIP primer consists of a polynucleotide represented by SEQ ID NO: 8, the F3 primer consists of a polynucleotide represented by SEQ ID NO: 31, and the B3 primer consists of a polynucleotide represented by SEQ ID NO: 32,
a primer set 4 wherein the FIP primer consists of a polynucleotide represented by SEQ ID NO: 9, the BIP primer consists of a polynucleotide represented by SEQ ID NO: 10, the F3 primer consists of a polynucleotide represented by SEQ ID NO: 31, and the B3 primer consists of a polynucleotide represented by SEQ ID NO: 32,
a primer set 11 wherein the FIP primer consists of a polynucleotide represented by SEQ ID NO: 7, the BIP primer consists of a polynucleotide represented by SEQ ID NO: 121, the F3 primer consists of a polynucleotide represented by SEQ ID NO: 31, and the B3 primer consists of a polynucleotide represented by SEQ ID NO: 123, and
a primer set 12 wherein the FIP primer consists of a polynucleotide represented by SEQ ID NO: 9, the BIP primer consists of a polynucleotide represented by SEQ ID NO: 122, the F3 primer consists of a polynucleotide represented by SEQ ID NO: 31, and the B3 primer consists of a polynucleotide represented by SEQ ID NO: 124,
which are used for amplification of a nucleotide sequence region containing nucleotide sequences coding for amino acids at positions 204 and 236 in the polymerase region of hepatitis B virus;
a primer set 5 wherein the FIP primer consists of a polynucleotide represented by SEQ ID NO: 15, the BIP primer consists of a polynucleotide represented by SEQ ID NO: 16, the F3 primer consists of a polynucleotide represented by SEQ ID NO: 33, and the B3 primer consists of a polynucleotide represented by SEQ ID NO: 34,
a primer set 6 wherein the FIP primer consists of a polynucleotide represented by SEQ ID NO: 17, the BIP primer consists of a polynucleotide represented by SEQ ID NO: 18, the F3 primer consists of a polynucleotide represented by SEQ ID NO: 35, and the B3 primer consists of a polynucleotide represented by SEQ ID NO: 36,
a primer set 7 wherein the FIP primer consists of a polynucleotide represented by SEQ ID NO: 19, the BIP primer consists of a polynucleotide represented by SEQ ID NO: 20, the F3 primer consists of a polynucleotide represented by SEQ ID NO: 37, and the B3 primer consists of a polynucleotide represented by SEQ ID NO: 38, and
a primer set 8 wherein the FIP primer consists of a polynucleotide represented by SEQ ID NO: 21, the BIP primer consists of a polynucleotide represented by SEQ ID NO: 22, the F3 primer consists of a polynucleotide represented by SEQ ID NO: 39, and the B3 primer consists of a polynucleotide represented by SEQ ID NO: 40,
which are used for amplification of a nucleotide sequence region containing a nucleotide sequence coding for an amino acid at position 236 in the polymerase region of hepatitis B virus; and
a primer set 9 wherein the FIP primer consists of a polynucleotide represented by SEQ ID NO: 23, the BIP primer consists of a polynucleotide represented by SEQ ID NO: 24, the F3 primer consists of a polynucleotide represented by SEQ ID NO: 41, and the B3 primer consists of a polynucleotide represented by SEQ ID NO: 42, and
a primer set 10 wherein the FIP primer consists of a polynucleotide represented by SEQ ID NO: 25, the BIP primer consists of a polynucleotide represented by SEQ ID NO: 26, the F3 primer consists of a polynucleotide represented by SEQ ID NO: 43, and the B3 primer consists of a polynucleotide represented by SEQ ID NO: 44,
which are used for amplification of a nucleotide sequence region containing a nucleotide sequence coding for an amino acid at position 181 in the polymerase region of hepatitis B virus.

4. The method according to claim 3, wherein the probe containing a polynucleotide derived from a drug-resistant strain and/or the probe containing a polynucleotide derived from a drug-nonresistant strain is at least one probe group which depending on the primer set used, is selected from the group consisting of:
a probe group 1 comprising at least one probe selected from the group consisting of a probe containing a polynucleotide represented by SEQ ID NO: 50 or its complementary strand, a probe containing a polynucleotide represented by SEQ ID NO: 51 or its complementary strand, a probe containing a polynucleotide represented by SEQ ID NO: 52 or its complementary strand, a probe containing a polynucleotide represented by SEQ ID NO: 53 or its complementary strand, a probe containing a polynucleotide represented by SEQ ID NO: 54 or its complementary strand, a probe containing a polynucleotide represented by SEQ ID NO: 55 or its complementary strand, a probe containing a polynucleotide represented by SEQ ID NO: 56 or its complementary strand, and a probe containing a polynucleotide represented by SEQ ID NO: 57 or its complementary strand,
a probe group 2 comprising at least one probe selected from the group consisting of a probe containing a polynucleotide represented by SEQ ID NO: 58 or its complementary strand, a probe containing a polynucleotide represented by SEQ ID NO: 59 or its complementary strand, a probe containing a polynucleotide represented by SEQ ID NO: 60 or its complementary strand, a probe containing a polynucleotide represented by SEQ ID NO: 61 or its complementary strand, a probe containing a polynucleotide represented by SEQ ID NO: 62 or its complementary strand, a probe containing a polynucleotide represented by SEQ ID NO: 63 or its complementary strand, a probe containing a polynucleotide represented by SEQ ID NO: 64 or its complementary strand, a probe containing a polynucleotide represented by SEQ ID NO: 65 or its complementary strand, a probe containing a polynucleotide represented by SEQ ID NO: 66 or its complementary strand, a probe containing a polynucleotide represented by SEQ ID NO: 67 or its complementary strand, a probe containing a polynucleotide represented by SEQ ID NO: 68 or its complementary strand, a probe containing a polynucleotide represented by SEQ ID NO: 69 or its complementary strand, a probe containing a polynucleotide represented by SEQ ID NO: 70 or its complementary strand, a probe containing a polynucleotide represented by SEQ ID NO: 71 or its complementary strand, a probe containing a polynucleotide represented by SEQ ID NO: 72 or its complementary strand, a probe containing a polynucleotide represented by SEQ ID NO: 73 or its complementary strand, a probe containing a polynucleotide represented by SEQ ID NO: 74 or its complementary strand, and a probe containing a polynucleotide represented by SEQ ID NO: 75 or its complementary strand, and
a probe group 3 comprising at least one probe selected from the group consisting of a probe containing a polynucleotide represented by SEQ ID NO: 76 or its complementary strand, a probe containing a polynucleotide represented by SEQ ID NO: 77 or its complementary strand, a probe containing a polynucleotide represented by SEQ ID NO: 78 or its complementary strand, a probe containing a polynucleotide represented by SEQ ID NO: 79 or its complementary strand, a probe containing a polynucleotide represented by SEQ ID NO: 80 or its complementary strand, a probe containing a polynucleotide represented by SEQ ID NO: 81 or its complementary strand, a probe containing a polynucleotide represented by SEQ ID NO: 82 or its complementary strand, a probe containing a polynucleotide represented by SEQ ID NO: 83 or its complementary strand, a probe containing a polynucleotide represented by SEQ ID NO: 84 or its complementary strand, a probe containing a polynucleotide represented by SEQ ID NO: 85 or its complementary strand, a probe containing a polynucleotide represented by SEQ ID NO: 86 or its complementary strand, a probe containing a polynucleotide represented by SEQ ID NO: 87 or its complementary strand, a probe containing a polynucleotide represented by SEQ ID NO: 88 or its complementary strand, a probe containing a polynucleotide represented by SEQ ID NO: 89 or its complementary strand, a probe containing a polynucleotide represented by SEQ ID NO: 90 or its complementary strand, a probe containing a polynucleotide represented by SEQ ID NO: 91 or its complementary strand, a probe containing a polynucleotide represented by SEQ ID NO: 132 or its complementary strand, a probe containing a polynucleotide represented by SEQ ID NO: 133 or its complementary strand, a probe containing a polynucleotide represented by SEQ ID NO: 134 or its complementary strand, a probe containing a polynucleotide represented by SEQ ID NO: 135 or its complementary strand, and a probe containing a polynucleotide represented by SEQ ID NO: 136 or its complementary strand, and each of the probes is 15 to 45 bases in full length.

5. The method according to claim 4, wherein the primer set is at least one primer set selected from the group consisting of:
a primer set 1 wherein the FIP primer consists of a polynucleotide represented by SEQ ID NO: 3, the BIP primer consists of a polynucleotide represented by SEQ ID NO: 4, the F3 primer consists of a polynucleotide represented by SEQ ID NO: 27, and the B3 primer consists of a polynucleotide represented by SEQ ID NO: 28,
a primer set 2 wherein the FIP primer consists of a polynucleotide represented by SEQ ID NO: 5, the BIP primer consists of a polynucleotide represented by SEQ ID NO: 6, the F3 primer consists of a polynucleotide represented by SEQ ID NO: 29, and the B3 primer consists of a polynucleotide represented by SEQ ID NO: 30,
a primer set 11 wherein the FIP primer consists of a polynucleotide represented by SEQ ID NO: 7, the BIP primer consists of a polynucleotide represented by SEQ ID NO: 121, the F3 primer consists of a polynucleotide represented by SEQ ID NO: 31, and the B3 primer consists of a polynucleotide represented by SEQ ID NO: 123, and
a primer set 12 wherein the FIP primer consists of a polynucleotide represented by SEQ ID NO: 9, the BIP primer consists of a polynucleotide represented by SEQ ID NO: 122, the F3 primer consists of a polynucleotide represented by SEQ ID NO: 31, and the B3 primer consists of a polynucleotide represented by SEQ ID NO: 124, and
the probe containing a polynucleotide derived from a drug-resistant strain and/or the probe containing a polynucleotide derived from a drug-nonresistant strain is a probe group comprising at least one probe selected from the group consisting of a probe containing a polynucleotide represented by SEQ ID NO: 93 or its complementary strand, a probe containing a polynucleotide represented by SEQ ID NO: 96 or its complementary strand, a probe containing a polynucleotide represented by SEQ ID NO: 99 or its complementary strand, a probe containing a polynucleotide represented by SEQ ID NO: 105 or its complementary strand, a probe containing a polynucleotide represented by SEQ ID NO: 108 or its complementary strand, a probe containing a polynucleotide represented by SEQ ID NO: 113 or its complementary strand, a probe containing a polynucleotide represented by SEQ ID NO: 117 or its complementary strand, a probe containing a polynucleotide represented by SEQ ID NO: 119 or its complementary strand, a probe containing a polynucleotide represented by SEQ ID NO: 147 or its complementary strand, a probe containing a polynucleotide represented by SEQ ID NO: 149 or its complementary strand, a probe containing a polynucleotide represented by SEQ ID NO: 152 or its complementary strand, a probe containing a polynucleotide represented by SEQ ID NO: 155 or its complementary strand, a probe containing a polynucleotide represented by SEQ ID NO: 157 or its complementary strand, a probe containing a polynucleotide represented by SEQ ID NO: 158 or its complementary strand, a probe containing a polynucleotide represented by SEQ ID NO: 161 or its complementary strand, a probe containing a polynucleotide represented by SEQ ID NO: 164 or its complementary strand, a probe containing a polynucleotide represented by SEQ ID NO: 167 or its complementary strand, a probe containing a polynucleotide represented by SEQ ID NO: 169 or its complementary strand, a probe containing a polynucleotide represented by SEQ ID NO: 171 or its complementary strand, a probe containing a polynucleotide represented by SEQ ID NO: 173 or its complementary strand, a probe containing a polynucleotide represented by SEQ ID NO: 174 or its complementary strand, a probe containing a polynucleotide represented by SEQ ID NO: 176 or its complementary strand, a probe containing a polynucleotide represented by SEQ ID NO: 179 or its complementary strand, a probe containing a polynucleotide represented by SEQ ID NO: 181 or its complementary strand, a probe containing a polynucleotide represented by SEQ ID NO: 183 or its complementary strand, a probe containing a polynucleotide represented by SEQ ID NO: 184 or its complementary strand, a probe containing a polynucleotide represented by SEQ ID NO: 186 or its complementary strand, a probe containing a polynucleotide represented by SEQ ID NO: 188 or its complementary strand, a probe containing a polynucleotide represented by SEQ ID NO: 190 or its complementary strand, a probe containing a polynucleotide represented by SEQ ID NO: 192 or its complementary strand, a probe containing a polynucleotide represented by SEQ ID NO: 193 or its complementary strand, and a probe containing a polynucleotide represented by SEQ ID NO: 195 or its complementary strand.

6. The method according to claim 3, wherein the step of obtaining the amplification product comprises:
a step of using the primer sets 1 and 2 simultaneously to amplify a hepatitis B virus nucleic acid in a sample solution by LAMP to yield first amplification products,
a step of using the primer sets 3 and 4 simultaneously to amplify an HBV nucleic acid in a sample solution by LAMP to yield second amplification products,
wherein the step of detecting a drug-resistant strain of the hepatitis B virus comprises hybridizing the first and second amplification products with a probe containing a polynucleotide derived from a drug-resistant strain and/or a probe containing a polynucleotide derived from a drug-nonresistant strain.

7. The method according to claim 3, wherein the step of obtaining the amplification product comprises:
a step of using the primer sets 1 and 2 simultaneously to amplify a hepatitis B virus nucleic acid in a sample solution by LAMP to yield first amplification products,
a step of using the primer sets 5 and 6 simultaneously to amplify an HBV nucleic acid in a sample solution by LAMP to yield second amplification products,
wherein the step of detecting a drug-resistant strain of the hepatitis B virus comprises hybridizing the first and second amplification products with a probe containing a polynucleotide derived from a drug-resistant strain and/or a probe containing a polynucleotide derived from a drug-nonresistant strain.

8. The method according to claim 3, wherein the step of obtaining the amplification product comprises:.
a step of using the primer sets 1 and 2 simultaneously to amplify a hepatitis B virus nucleic acid in a sample solution by LAMP to yield first amplification products,
a step of using the primer sets 7 and 8 simultaneously to amplify an HBV nucleic acid in a sample solution by LAMP to yield second amplification products,
wherein the step of detecting a drug-resistant strain of the hepatitis B virus comprises hybridizing the first and second amplification products with a probe containing a polynucleotide derived from a drug-resistant strain and/or a probe containing a polynucleotide derived from a drug-nonresistant strain.

9. The method according to claim 3, wherein the step of obtaining the amplification product comprises:
a step of using the primer sets 3 and 4 simultaneously to amplify a hepatitis B virus nucleic acid in a sample solution by LAMP to yield first amplification products,
a step of using the primer sets 9 and 10 simultaneously to amplify an HBV nucleic acid in a sample solution by LAMP to yield second amplification products,
wherein the step of detecting a drug-resistant strain of the hepatitis B virus comprises hybridizing the first and second amplification products with a probe containing a polynucleotide derived from a drug-resistant strain and/or a probe containing a polynucleotide derived from a drug-nonresistant strain.

10. The method according to claim 3, wherein the step of obtaining the amplification product comprises:
a step of using the primer sets 1 and 2 simultaneously to amplify a hepatitis B virus nucleic acid in a sample solution by LAMP to yield first amplification products,
a step of using the primer sets 11 and 12 simultaneously to amplify an HBV nucleic acid in a sample solution by LAMP to yield second amplification products,
wherein the step of detecting a drug-resistant strain of the hepatitis B virus comprises hybridizing the first and second amplification products with a probe containing a polynucleotide derived from a drug-resistant strain and/or a probe containing a polynucleotide derived from a drug-nonresistant strain.

11. The method according to claim 3, wherein the step of obtaining the amplification product comprises:
a step of using the primer sets 9 and 10 simultaneously to amplify a hepatitis B virus nucleic acid in a sample solution by LAMP to yield first amplification products,
a step of using the primer sets 11 and 12 simultaneously to amplify an HBV nucleic acid in a sample solution by LAMP to yield second amplification products,
wherein the step of detecting a drug-resistant strain of the hepatitis B virus comprises hybridizing the first and second amplification products with a probe containing a polynucleotide derived from a drug-resistant strain and/or a probe containing a polynucleotide derived from a drug-nonresistant strain.

12. An assay kit for detecting a drug-resistant or drug-nonresistant strain of hepatitis B virus, comprising:
the primer set of claim 1, and
a probe containing a polynucleotide derived from a drug-resistant strain of hepatitis B virus and/or a probe containing a polynucleotide derived from a drug-nonresistant strain of hepatitis B virus.

13. The assay kit for detection of the drug resistance of hepatitis B virus whose genotype is type C, wherein the probe containing a polynucleotide derived from a drug-resistant strain of hepatitis B virus and/or the probe containing a polynucleotide derived from a drug-nonresistant strain of hepatitis B virus comprises at least one probe group which depending on the primer set used, is selected from the group consisting of:
a probe group 1 comprising at least one probe selected from the group consisting of a probe containing a polynucleotide represented by SEQ ID NO: 50 or its complementary strand, a probe containing a polynucleotide represented by SEQ ID NO: 51 or its complementary strand, a probe containing a polynucleotide represented by SEQ ID NO: 52 or its complementary strand, a probe containing a polynucleotide represented by SEQ ID NO: 53 or its complementary strand, a probe containing a polynucleotide represented by SEQ ID NO: 54 or its complementary strand, a probe containing a polynucleotide represented by SEQ ID NO: 55 or its complementary strand, a probe containing a polynucleotide represented by SEQ ID NO: 56 or its complementary strand, and a probe containing a polynucleotide represented by SEQ ID NO: 57 or its complementary strand,
a probe group 2 comprising at least one probe selected from the group consisting of a probe containing a polynucleotide represented by SEQ ID NO: 58 or its complementary strand, a probe containing a polynucleotide represented by SEQ ID NO: 59 or its complementary strand, a probe containing a polynucleotide represented by SEQ ID NO: 60 or its complementary strand, a probe containing a polynucleotide represented by SEQ ID NO: 61 or its complementary strand, a probe containing a polynucleotide represented by SEQ ID NO: 62 or its complementary strand, a probe containing a polynucleotide represented by SEQ ID NO: 63 or its complementary strand, a probe containing a polynucleotide represented by SEQ ID NO: 64 or its complementary strand, a probe containing a polynucleotide represented by SEQ ID NO: 65 or its complementary strand, a probe containing a polynucleotide represented by SEQ ID NO: 66 or its complementary strand, a probe containing a polynucleotide represented by SEQ ID NO: 67 or its complementary strand, a probe containing a polynucleotide represented by SEQ ID NO: 68 or its complementary strand, a probe containing a polynucleotide represented by SEQ ID NO: 69 or its complementary strand, a probe containing a polynucleotide represented by SEQ ID NO: 70 or its complementary strand, a probe containing a polynucleotide represented by SEQ ID NO: 71 or its complementary strand, a probe containing a polynucleotide represented by SEQ ID NO: 72 or its complementary strand, a probe containing a polynucleotide represented by SEQ ID NO: 73 or its complementary strand, a probe containing a polynucleotide represented by SEQ ID NO: 74 or its complementary strand, and a probe containing a polynucleotide represented by SEQ ID NO: 75 or its complementary strand, and
a probe group 3 comprising at least one probe selected from the group consisting of a probe containing a polynucleotide represented by SEQ ID NO: 76 or its complementary strand, a probe containing a polynucleotide represented by SEQ ID NO: 77 or its complementary strand, a probe containing a polynucleotide represented by SEQ ID NO: 78 or its complementary strand, a probe containing a polynucleotide represented by SEQ ID NO: 79 or its complementary strand, a probe containing a polynucleotide represented by SEQ ID NO: 80 or its complementary strand, a probe containing a polynucleotide represented by SEQ ID NO: 81 or its complementary strand, a probe containing a polynucleotide represented by SEQ ID NO: 82 or its complementary strand, a probe containing a polynucleotide represented by SEQ ID NO: 83 or its complementary strand, a probe containing a polynucleotide represented by SEQ ID NO: 84 or its complementary strand, a probe containing a polynucleotide represented by SEQ ID NO: 85 or its complementary strand, a probe containing a polynucleotide represented by SEQ ID NO: 86 or its complementary strand, a probe containing a polynucleotide represented by SEQ ID NO: 87 or its complementary strand, a probe containing a polynucleotide represented by SEQ ID NO: 88 or its complementary strand, a probe containing a polynucleotide represented by SEQ ID NO: 89 or its complementary strand, a probe containing a polynucleotide represented by SEQ ID NO: 90 or its complementary strand, a probe containing a polynucleotide represented by SEQ ID NO: 91 or its complementary strand, a probe containing a polynucleotide represented by SEQ ID NO: 132 or its complementary strand, a probe containing a polynucleotide represented by SEQ ID NO: 133 or its complementary strand, a probe containing a polynucleotide represented by SEQ ID NO: 134 or its complementary strand, a probe containing a polynucleotide represented by SEQ ID NO: 135 or its complementary strand, and a probe containing a polynucleotide represented by SEQ ID NO: 136 or its complementary strand, and each of the probes is 15 to 45 bases in full length.

14. The assay kit for detection of the drug resistance of hepatitis B virus whose genotype is type C, wherein the primer set is at least one primer set selected from the group consisting of:
a primer set 1 wherein the FIP primer consists of a polynucleotide represented by SEQ ID NO: 3, the BIP primer consists of a polynucleotide represented by SEQ ID NO: 4, the F3 primer consists of a polynucleotide represented by SEQ ID NO: 27, and the B3 primer consists of a polynucleotide represented by SEQ ID NO: 28,
a primer set 2 wherein the FIP primer consists of a polynucleotide represented by SEQ ID NO: 5, the BIP primer consists of a polynucleotide represented by SEQ ID NO: 6, the F3 primer consists of a polynucleotide represented by SEQ ID NO: 29, and the B3 primer consists of a polynucleotide represented by SEQ ID NO: 30,
a primer set 3 wherein the FIP primer consists of a polynucleotide represented by SEQ ID NO: 7, the BIP primer consists of a polynucleotide represented by SEQ ID NO: 8, the F3 primer consists of a polynucleotide represented by SEQ ID NO: 31, and the B3 primer consists of a polynucleotide represented by SEQ ID NO: 32,
a primer set 4 wherein the FIP primer consists of a polynucleotide represented by SEQ ID NO: 9, the BIP primer consists of a polynucleotide represented by SEQ ID NO: 10, the F3 primer consists of a polynucleotide represented by SEQ ID NO: 31, and the B3 primer consists of a polynucleotide represented by SEQ ID NO: 32,
a primer set 11 wherein the FIP primer consists of a polynucleotide represented by SEQ ID NO: 7, the BIP primer consists of a polynucleotide represented by SEQ ID NO: 121, the F3 primer consists of a polynucleotide represented by SEQ ID NO: 31, and the B3 primer consists of a polynucleotide represented by SEQ ID NO: 123, and
a primer set 12 wherein the FIP primer consists of a polynucleotide represented by SEQ ID NO: 9, the BIP primer consists of a polynucleotide represented by SEQ ID NO: 122, the F3 primer consists of a polynucleotide represented by SEQ ID NO: 31, and the B3 primer consists of a polynucleotide represented by SEQ ID NO: 124, and
the probe containing a polynucleotide derived from a drug-resistant strain of hepatitis B virus and/or the probe containing a polynucleotide derived from a drug-nonresistant strain of hepatitis B virus is a group consisting of a probe consisting of a polynucleotide represented by SEQ ID NO: 93 or its complementary strand, a probe consisting of a polynucleotide represented by SEQ ID NO: 96 or its complementary strand, a probe consisting of a polynucleotide represented by SEQ ID NO: 99 or its complementary strand, a probe consisting of a polynucleotide represented by SEQ ID NO: 105 or its complementary strand, a probe consisting of a polynucleotide represented by SEQ ID NO: 108 or its complementary strand, a probe consisting of a polynucleotide represented by SEQ ID NO: 113 or its complementary strand, a probe consisting of a polynucleotide represented by SEQ ID NO: 117 or its complementary strand, a probe consisting of a polynucleotide represented by SEQ ID NO: 119 or its complementary strand, a probe consisting of a polynucleotide represented by SEQ ID NO: 147 or its complementary strand, a probe consisting of a polynucleotide represented by SEQ ID NO: 149 or its complementary strand, a probe consisting of a polynucleotide represented by SEQ ID NO: 152 or its complementary strand, a probe consisting of a polynucleotide represented by SEQ ID NO: 155 or its complementary strand, a probe consisting of a polynucleotide represented by SEQ ID NO: 157 or its complementary strand, a probe consisting of a polynucleotide represented by SEQ ID NO: 158 or its complementary strand, a probe consisting of a polynucleotide represented by SEQ ID NO: 161 or its complementary strand, a probe consisting of a polynucleotide represented by SEQ ID NO: 164 or its complementary strand, a probe consisting of a polynucleotide represented by SEQ ID NO: 167 or its complementary strand, a probe consisting of a polynucleotide represented by SEQ ID NO: 169 or its complementary strand, a probe consisting of a polynucleotide represented by SEQ ID NO: 171 or its complementary strand, a probe consisting of a polynucleotide represented by SEQ ID NO: 173 or its complementary strand, a probe consisting of a polynucleotide represented by SEQ ID NO: 174 or its complementary strand, a probe consisting of a polynucleotide represented by SEQ ID NO: 176 or its complementary strand, a probe consisting of a polynucleotide represented by SEQ ID NO: 179 or its complementary strand, a probe consisting of a polynucleotide represented by SEQ ID NO: 181 or its complementary strand, a probe consisting of a polynucleotide represented by SEQ ID NO: 183 or its complementary strand, a probe consisting of a polynucleotide represented by SEQ ID NO: 184 or its complementary strand, a probe consisting of a polynucleotide represented by SEQ ID NO: 186 or its complementary strand, a probe consisting of a polynucleotide represented by SEQ ID NO: 188 or its complementary strand, a probe consisting of a polynucleotide represented by SEQ ID NO: 190 or its complementary strand, a probe consisting of a polynucleotide represented by SEQ ID NO: 192 or its complementary strand, a probe consisting of a polynucleotide represented by SEQ ID NO: 193 or its complementary strand, and a probe consisting of a polynucleotide represented by SEQ ID NO: 195 or its complementary strand.

15. The primer set according to claim 1, wherein the primer set is at least one set from among the primer sets 1, 3, 5, 7, 9 and 11 and is used in detection of the drug resistance of hepatitis B virus whose genotype is type B.

16. The primer set according to claim 1, wherein the primer set is at least one set from among the primer sets 2, 4, 6, 8, 10 and 12 and is used in detection of the drug resistance of hepatitis B virus whose genotype is type C.

17. An assay kit for detection of the drug resistance of hepatitis B virus whose genotype is type B, comprising:
the primer set of claim 10, and
a probe group containing a polynucleotide represented by SEQ ID NO: 50 or its complementary strand, a polynucleotide represented by SEQ ID NO: 51 or its complementary strand, a polynucleotide represented by SEQ ID NO: 52 or its complementary strand, a polynucleotide represented by SEQ ID NO: 53 or its complementary strand, a polynucleotide represented by SEQ ID NO: 54 or its complementary strand, a polynucleotide represented by SEQ ID NO: 55 or its complementary strand, a polynucleotide represented by SEQ ID NO: 56 or its complementary strand, a polynucleotide represented by SEQ ID NO: 57 or its complementary strand, a polynucleotide represented by SEQ ID NO: 58 or its complementary strand, a polynucleotide represented by SEQ ID NO: 59 or its complementary strand, a polynucleotide represented by SEQ ID NO: 60 or its complementary strand, a polynucleotide represented by SEQ ID NO: 61 or its complementary strand, a polynucleotide represented by SEQ ID NO: 62 or its complementary strand, a polynucleotide represented by SEQ ID NO: 63 or its complementary strand, a polynucleotide represented by SEQ ID NO: 76 or its complementary strand, a polynucleotide represented by SEQ ID NO: 77 or its complementary strand, a polynucleotide represented by SEQ ID NO: 78 or its complementary strand, a polynucleotide represented by SEQ ID NO: 79 or its complementary strand, a polynucleotide represented by SEQ ID NO: 80 or its complementary strand, a polynucleotide represented by SEQ ID NO: 81 or its complementary strand, a polynucleotide represented by SEQ ID NO: 82 or its complementary strand, a polynucleotide represented by SEQ ID NO: 83 or its complementary strand, a polynucleotide represented by SEQ ID NO: 132 or its complementary strand, a polynucleotide represented by SEQ ID NO: 133 or its complementary strand, and a polynucleotide represented by SEQ ID NO: 134 or its complementary strand, a polynucleotide represented by SEQ ID NO: 135 or its complementary strand, and a polynucleotide represented by SEQ ID NO: 136 or its complementary strand, each of which is 15 to 45 bases in full length.

18. An assay kit for detection of the drug resistance of hepatitis B virus whose genotype is type C, comprising:
the primer set of claim 11, and
a probe group containing a polynucleotide represented by SEQ ID NO: 50 or its complementary strand, a polynucleotide represented by SEQ ID NO: 51 or its complementary strand, a polynucleotide represented by SEQ ID NO: 52 or its complementary strand, a polynucleotide represented by SEQ ID NO: 53 or its complementary strand, a polynucleotide represented by SEQ ID NO: 54 or its complementary strand, a polynucleotide represented by SEQ ID NO: 55 or its complementary strand, a polynucleotide represented by SEQ ID NO: 56 or its complementary strand, a polynucleotide represented by SEQ ID NO: 57 or its complementary strand, a polynucleotide represented by SEQ ID NO: 64 or its complementary strand, a polynucleotide represented by SEQ ID NO: 65 or its complementary strand, a polynucleotide represented by SEQ ID NO: 66 or its complementary strand, a polynucleotide represented by SEQ ID NO: 67 or its complementary strand, a polynucleotide represented by SEQ ID NO: 68 or its complementary strand, a polynucleotide represented by SEQ ID NO: 69 or its complementary strand, a polynucleotide represented by SEQ ID NO: 70 or its complementary strand, a polynucleotide represented by SEQ ID NO: 71 or its complementary strand, a polynucleotide represented by SEQ ID NO: 72 or its complementary strand, a polynucleotide represented by SEQ ID NO: 73 or its complementary strand, a polynucleotide represented by SEQ ID NO: 74 or its complementary strand, a polynucleotide represented by SEQ ID NO: 75 or its complementary strand, a polynucleotide represented by SEQ ID NO: 84 or its complementary strand, a polynucleotide represented by SEQ ID NO: 85 or its complementary strand, a polynucleotide represented by SEQ ID NO: 86 or its complementary strand, a polynucleotide represented by SEQ ID NO: 87 or its complementary strand, a polynucleotide represented by SEQ ID NO: 88 or its complementary strand, a polynucleotide represented by SEQ ID NO: 89 or its complementary strand, a polynucleotide represented by SEQ ID NO: 90 or its complementary strand, a polynucleotide represented by SEQ ID NO: 91 or its complementary strand, a polynucleotide represented by SEQ ID NO: 132 or its complementary strand, a polynucleotide represented by SEQ ID NO: 133 or its complementary strand, a polynucleotide represented by SEQ ID NO: 134 or its complementary strand, a polynucleotide represented by SEQ ID NO: 135 or its complementary strand, and a polynucleotide represented by SEQ ID NO: 136 or its complementary strand, each of which is 15 to 45 bases in full length.
